(19) United States
(12) Patent Application Publication
(10) Pub. No.: US 2023/0040909 A1
(43) Pub. Date: Feb. 9, 2023

(54) INDOLE CARBOXAMIDE COMPOUNDS AND USE THEREOF FOR THE TREATMENT OF MYCOBACTERIAL INFECTIONS

(71) Applicant: THE GLOBAL ALLIANCE FOR TB DRUG DEVELOPMENT, INC., New York, NY (US)

(72) Inventors: Takushi KANEKO, Guilford, CT (US); Nader FOTOUHI, Basking Ridge, NJ (US)

(21) Appl. No.: 17/761,716

(22) PCT Filed: Sep. 25, 2020
(86) PCT No.: PCT/US20/52938

§ 371 Date: Mar. 18, 2022

Related U.S. Application Data
(60) Provisional application No. 62/906,424, filed on Sep. 26, 2019.

(51) Int. Cl. A61K 31/695; C07F 7/08; A61P 31/06; A61K 31/496; A61K 31/438; A61K 31/4409; A61K 31/133; A61K 31/7036; A61K 31/498; A61K 31/42; A61K 31/5377; A61K 31/4709

(52) U.S. Cl. CPC A61K 31/695; C07F 7/0816; A61P 31/06; A61K 31/496; A61K 31/438; A61K 31/4409; A61K 31/133; A61K 31/7036; A61K 31/498; A61K 31/42; A61K 31/5377; A61K 31/4709

(57) ABSTRACT

Provided herein are compounds of Formula (I) as well as pharmaceutically acceptable salts thereof, wherein the substituents are as those disclosed in the specification. These compounds, and the pharmaceutical compositions containing them, are useful for the treatment of tuberculosis.

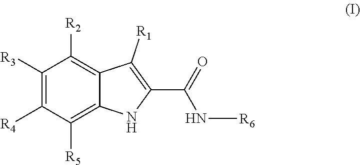

### FIELD OF THE INVENTION

The invention is directed, for example, to compounds of Formula (I):

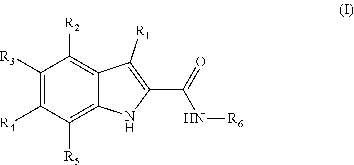

and to pharmaceutical compositions comprising the compounds. The compounds and compositions disclosed herein are antibacterials and are useful for the treatment of tuberculosis and other mycobacterial infections.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

Mycobacterium tuberculosis (“M. tb”) is the causative agent of tuberculosis (“TB”), a devastating infectious disease. It is estimated that about 2 million TB patients die each year globally. The treatment of drug-susceptible TB currently centers on four antibiotics, isoniazid, rifampicin, ethambutol, and pyrazinamide which were introduced more than 40 years ago (Franz 2017). Failure to properly treat tuberculosis has caused global drug resistance in Mtb and thus rendering some medications ineffective. A need exists in the art, therefore, to identify new chemical entities to treat TB.

### SUMMARY OF THE INVENTION

The present invention is directed to compounds of Formula (I):

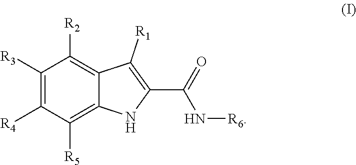

The present invention is also directed to pharmaceutical compositions containing the above compounds and to methods of treating microbial infections such as tuberculosis.

### BRIEF DESCRIPTION OF THE DRAWINGS

The drawings described below are for illustrative purposes only and are not intended to limit the scope of the invention.

FIG. 1 depicts the cardiovascular liabilities of select compounds.

### DETAILED DESCRIPTION OF THE INVENTION

It is to be understood that the terminology employed herein is for the purpose of describing particular embodiments, and is not intended to be limiting. Further, although any methods, devices and materials similar or equivalent to those described herein can be used in the practice or testing of the invention, certain methods, devices and materials are now described.

The present invention relates to novel indole carboxamide compounds, their preparations, and to their use as drugs for treating tuberculosis and other mycobacteria infections. The compounds, in certain embodiments, have the following general structure:

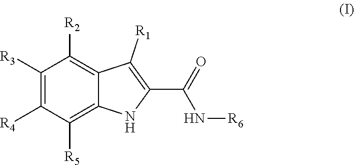

wherein

R₁ is hydrogen, lower alkyl, or halogen; and

R₂ is hydrogen, lower alkyl, halo, cyano, trifluoromethyl, halo-lower alkyl, di-halo-lower alkyl, lower alkoxy, —OCH₂CH₂OCH₃, or carboxamide; and

R₃ is hydrogen, lower alkyl, halo, cyano, trifluoromethyl, halo-lower alkyl, di-halo-lower alkyl, lower alkoxy, —OCH₂CH₂OCH₃, or carboxamide; and

R₄ is hydrogen, lower alkyl, halo, cyano, trifluoromethyl, halo-lower alkyl, di-halo-lower alkyl, alkoxy, —OCH₂CH₂OCH₃, —(O(CH₂)ₘₘ)ₙₙ-morpholinyl, piperidinyl, ((C₁-C₄)alkyl)NH—, or (phenyl)NH—, where mm is 1 or 2 and nn is 0 or 1 or carboxamide; or

R₃ and R₄ taken together with the aromatic carbon atoms to which they are attached form a fused 1,3-dioxolo; and

R₅ is hydrogen, lower alkyl, halo, cyano, trifluoromethyl, halo-lower alkyl, di-halo-lower alkyl, alkoxy, or carboxamide; and

R₆ is

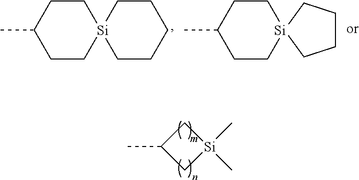

and

m is 1, 2 or 3,

n is 1, 2, 3, or 4.

In the case where m is not equal to n, there exists a stereocenter in the amine and in the resulting amide. The product may be a mixture or it may be resolved individual stereoisomers of the amide although the absolute stereochemical assignments are not made. Under such a case, a number (MPL-xxx) without a suffix A or B is meant for a racemic mixture whereas suffix A and B (such as MPL-xxxA and MPL-xxxB) is meant to indicate resolved enantiomers although no absolute configuration has been assigned to each enantiomer. Separation of stereoisomers are most effectively achieved by the use of Super Fluid Chromatography (SFC) equipped with a chiral column.

In one embodiment of the invention, the compounds of the invention can treat TB in combination with other anti-TB agents. The anti-TB agents include, but are not limited to, rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin, and levofloxacin.

### Definitions

As used herein, the term “alkyl”, alone or in combination with other groups, refers to a branched or straight-chain monovalent saturated aliphatic hydrocarbon radical of one to twenty carbon atoms, in one embodiment one to sixteen carbon atoms, in another embodiment one to ten carbon atoms.

The term “lower alkyl”, alone or in combination with other groups, refers to a branched or straight-chain alkyl radical of one to nine carbon atoms, in one embodiment one to six carbon atoms, in another embodiment one to four carbon atoms, in a further embodiment four to six carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

As used herein, the term “alkoxy” means alkyl-O—; and “alkoyl” means alkyl-CO—. Alkoxy substituent groups or alkoxy-containing substituent groups may be substituted by, for example, one or more alkyl or halo groups. “Lower alkoxy” as used herein denotes an alkoxy group with a “lower alkyl” group as previously defined.”

As used herein, the term “halogen” means a fluorine, chlorine, bromine or iodine radical, or in some embodiments a fluorine, chlorine or bromine radical.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, 1H-indenyl and the like.

The alkyl, lower alkyl and aryl groups may be substituted or unsubstituted. When substituted, there will generally be, for example, 1 to 4 substituents present. These substituents may optionally form a ring with the alkyl, lower alkyl or aryl group with which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl, in other embodiments, for example, methoxy and ethoxy), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

As would be readily understood from the disclosure provided herein, any reference to a group falling within a generic group may be substituted or unsubstituted in the same manner. For example, a phenyl group may be substituted in the same manner as an aryl group. The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. Examples of such groups include, but not limited to, pyridinyl, pyrazinyl, pyridazinyl, 1,2,3-triazinyl, 1,2,4-triazinyl, oxazolyl, thiazolyl, and the like.

In some instances, a term is preceded by “(C#-C#).” As would be readily understood from the disclosure provided herein, this defines the number of carbon atoms associated with the term. For example, (C1-C6)alkyl means an alkyl in which the branched or straight-chain monovalent saturated aliphatic hydrocarbon radical has one to 6 carbon atoms. As would be readily understood from the disclosure provided herein, all substitution definitions apply equally to these structures. For example, (C1-C6)alkyl may be substituted in the same manner an alkyl is substituted.

By any range disclosed herein, it is meant that all integer unit amounts within the range are specifically disclosed as part of the invention. Thus, for example, 1 to 12 units means that 1, 2, 3 . . . 12 units are included as embodiments of this invention.

As used herein, multi-drug-resistant tuberculosis (MDR-TB) is a form of TB which has resistance to isoniazid and rifampin, with or without resistance to other drugs. As used herein, pre-extensively drug resistant (Pre-XDR-TB) is a form of TB which has resistance to isoniazid and rifampin and either a fluoroquinolone or an injectable drug but not both. As used herein, extensively drug resistant tuberculosis (XDR-TB) is a form of TB which has resistance to isoniazid, rifampin, fluoroquinolones and at least one injectable drug (e.g., streptomycin, amikacin, kanamycin, capreomycin).

Compounds of the present invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

In one embodiment, the present invention also provides for combination therapy of the compounds of the present invention with at least one other therapeutic agent. The other agent may be prepared for simultaneous, separate or sequential use in therapy to treat the subject.

In the practice of the method of the present invention, an effective amount of any one of the compounds of this invention, or a combination of any of the compounds of this invention, is administered via any of the usual and acceptable methods known in the art, either singly or in combination. The compounds or compositions can thus be administered, for example, ocularly, orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are representative liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. In one embodiment, the therapeutically effective amount is in an amount of from about 10 mg to about 500 mg per day.

It will be appreciated that the compounds of the invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds of the present invention can be prepared beginning with commercially available starting materials and utilizing general synthetic techniques and procedures known to those skilled in the art. For example, general synthetic techniques disclosed in WO 2014/03137968 are known to those skilled in the art. Chemicals may be purchased from companies such, as for example, Aldrich, Argonaut Technologies, VWR and Lancaster. Chromatography supplies and equipment may be purchased from such companies as for example AnaLogix, Inc., Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

### Synthesis of Representative Compounds of the Invention

The compounds of the invention can be prepared according to the following Scheme showing general methods A and B:

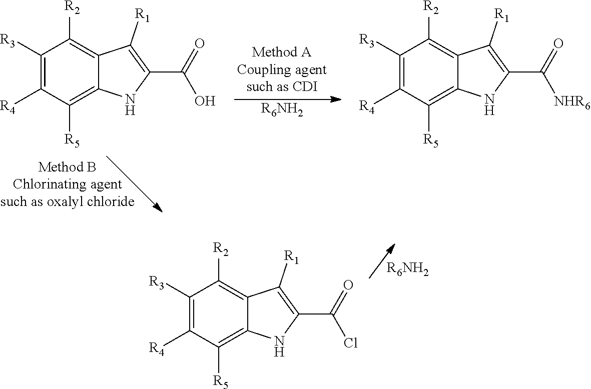

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Abbreviations used: ABPR, automatic back-pressure regulator; ACN, acetonitrile; aq., aqueous; CDI, 1,1′-carbonyl diimidazole; m-CPBA, meta-chloroperbenzoic acid; DCM, dichloromethane; DEA, diethyl amine; DME, dimethoxyethane; DMF, dimethylformamide; DMSO, dimethylsulfoxide; EDCI, 1-ethyl-3-(3-dimethylaminopropyl)carbodiimide; ESI, electrospray ionization; EtOAc, ethyl acetate; eq, equivalent; FA, formic acid; HOBt, 1-hydroxybenzonitrile; HPLC, high performance liquid chromatography; IPA, isopropyl alcohol; LAH, lithium aluminium hydride; LCMS or LC-MS, liquid chromatography-mass spectrometry; LDA, lithium diisopropylamide; min, minute; m/z, mass-to-charge ratio; NCS, N-chlorosuccinimide; nm, nanometer; NMR, nuclear magnetic resonance; ¹H NMR, proton NMR; Pd(dppf)Cl₂, 1,1′-Bis(diphenylphosphino)ferrocene]dichloropalladium(II); prep-HPLC, preparative HPLC; prep-TLC, preparative TLC; psi, pound per square inch; sat., saturated; SFC, supercritical fluid chromatography; TBAF, tetra-n-butylammonium fluoride; TEA, triethylamine; THF, tetrahydrofuran: TLC, thin-layer chromatography; TMEDA, tetramethylethylenediamine; TMS, trimethylsilyl; TMSCl, chloro(trimethyl)silane; Tos, p-tolylsulfonyl; TosCl, 4-toluenesulfonyl chloride; TOsOH, p-toluene sulfonic acid; ul, microliter; umol, micromole; um, micrometer; δ, chemical shift in ppm.

Reactions were monitored by TLC or LCMS and compounds were characterized by LCMS and/or NMR. Shimadzu LC20-MS2010 or LC20-MS2020 were used for LC/MS analysis. Varian 400 MHz, Varian 500 MHz or Bruker 500 MHz were used for NMR measurement.

General conditions for prep-HPLC purification: Instrument: Gilson GX281; Flow rate: 25 mL/min; Detector: UV 220 and UV 254

“[water (X)—Y]; B %: J %-K %, Lmin” stands for mobile phase A: X in water; B: Y; gradient J %-K % B over L min. For example, ‘[water (0.225% FA)-ACN]; B %: 36%-66%, 11 min’ means mobile phase: A: 0.025% formic acid in water, B: acetonitrile; gradient: 36%-66% B over 11 min.

### Example 1, MPL-196

### Synthesis of N-(1,1-dimethylsilinan-4-yl)-6-fluoro-4-methoxy-1H-indole-2-carboxamide

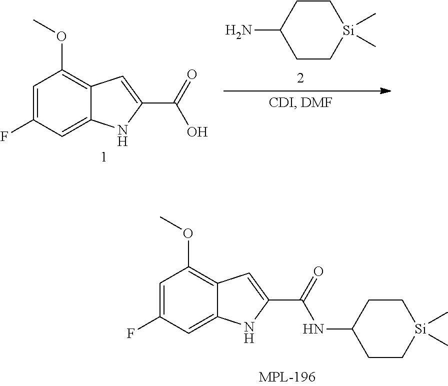

A solution of 6-fluoro-4-methoxy-1H-indole-2-carboxylic acid (160 mg, 764.91 μmol, 1 eq) and CDI (148.84 mg, 917.90 μmol, 1.2 eq) in DMF (2 mL) was stirred at 25° C. for 0.5 h. 1,1-dimethylsilinan-4-amine (131.54 mg, 917.90 μmol, 1.2 eq) was added. The mixture was stirred at 25° C. for 11.5 h. LCMS showed no starting material. TLC showed one spot was observed. The reaction mixture was added to water (20 ml), filtered and the filter cake was washed with 10 mL of water, dried in vacuo to give product. The residue was purified by column chromatography (SiO₂, Petroleum ether/EtOAc=1:0 to 10:1). The residue was diluted in ACN (5 mL) and H₂O (20 mL), then lyophilized. The product N-(1,1-dimethylsilinan-4-yl)-6-fluoro-4-methoxy-1H-indole-2-carboxamide (141.9 mg, 421.30 μmol, 55.08% yield, 99.3% purity) was obtained as white solid.

LCMS (ESI) m/z 335.2 [M+H]⁺

¹H NMR (500 MHz, DMSO-d6) δ=11.57 (s, 1H), 8.13 (d, J=8.1 Hz, 1H), 7.22 (d, J=1.7 Hz, 1H), 6.71 (dd, J=1.3, 9.5 Hz, 1H), 6.45 (dd, J=1.8, 12.1 Hz, 1H), 3.88 (s, 3H), 3.75-3.64 (m, 1H), 2.00-1.92 (m, 2H), 1.62-1.52 (m, 2H), 0.76 (br d, J=14.5 Hz, 2H), 0.59 (dt, J=4.7, 14.2 Hz, 2H), 0.08 (s, 3H), 0.03 (s, 3H).

### Example 2, MPL-203

### Synthesis of N-(1,1-dimethylsilinan-4-yl)-4,6-difluoro-1H-indole-2-carboxamide

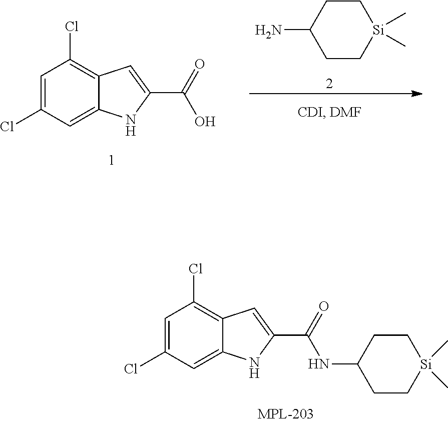

To a solution of 4, 6-dichloro-1H-indole-2-carboxylic acid (150 mg, 652.04 μmol, 1 eq) in DMF (4 mL) was added CDI (137.45 mg, 847.65 μmol, 1.3 eq). The mixture was stirred at 20° C. for 0.5 h. Then 1, 1-dimethylsilinan-4-amine (121.47 mg, 847.65 μmol, 1.3 eq) was added. The mixture was stirred at 20° C. for 11.5 h. LCMS showed there was no starting material. The reaction was added dropwise to H₂O (20 mL). There was much precipitation which was collected by filter. The cake was diluted with EtOAc (30 mL), dried with anhydrous MgSO₄, filtered. The filtrate was concentrated in vacuo. The residue was diluted in ACN (5 mL) and H₂O (20 mL), then lyophilized without further purification. Compound 4, 6-dichloro-N-(1,1-dimethylsilinan-4-yl)-1H-Indole-2-carboxamide (127.3 mg, 351.09 μmol, 53.85% yield, 98.001% purity) was obtained as a white solid.

LCMS (ESI), m/z 355.2[M+H]⁺

¹H NMR (500 MHz, DMSO-d6) δ=12.03 (br s, 1H), 8.44 (br d, J=8.2 Hz, 1H), 7.41 (s, 1H), 7.29 (s, 1H), 7.21 (d, J=1.5 Hz, 1H), 3.77-3.67 (m, 1H), 2.03-1.95 (m, 2H), 1.65-1.54 (m, 2H), 0.77 (br d, J=14.5 Hz, 2H), 0.61 (dt, J=4.7, 14.2 Hz, 2H), 0.11-0.01 (m, 6H).

### Example 3, MPL-204

### Synthesis of N-(1,1-dimethylsilinan-4-yl)-4,6-difluoro-1H-indole-2-carboxamide

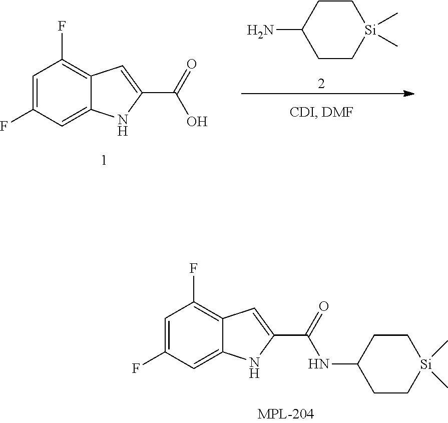

To a solution of 4, 6-difluoro-1H-indole-2-carboxylic acid (2 g, 10.15 mmol, 1 eq) in DMF (40 mL) was added CDI (1.81 g, 11.16 mmol, 1.1 eq). The mixture was stirred at 30° C. for 0.5 h. Then 1, 1-dimethylsilinan-4-amine (1.60 g, 11.16 mmol, 1.1 eq) was added. The mixture was stirred at 30° C. for 11.5 h. LCMS showed there was no starting material. The reaction was added dropwise to H₂O (300 mL). There was much precipitation which was collected by filter. The cake was transferred in bottom flask. The crude product was triturated with ACN (20 mL) at 30° C. for 45 min. Compound N-(1,1-dimethylsilinan-4-yl)-4,6-difluoro-1H-indole-2-carboxamide (2.74 g, 8.15 mmol, 80.29% yield, 95.752% purity) was obtained as a white solid.

LCMS (ESI), m/z 323[M+H]⁺

¹H NMR (400 MHz, DMSO-d6) δ=11.94 (br s, 1H), 8.30 (br d, J=8.1 Hz, 1H), 7.25 (s, 1H), 7.01 (br d, J=8.4 Hz, 1H), 6.90-6.83 (m, 1H), 3.77-3.64 (m, 1H), 2.05-1.93 (m, 2H), 1.65-1.52 (m, 2H), 0.83-0.73 (m, 2H), 0.61 (dt, J=4.6, 14.1 Hz, 2H), 0.09 (s, 3H), 0.03 (s, 3H).

### Example 4, MPL-251

### Synthesis of 4,6-difluoro-N-(5-silaspiro[4.5]decan-8-yl)-1H-indole-2-carboxamide

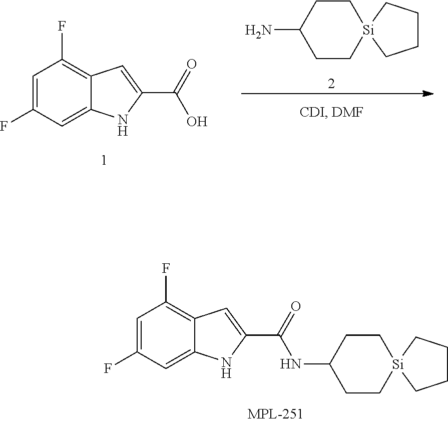

To a solution of 4,6-difluoro-1H-indole-2-carboxylic acid (50 mg, 253.63 μmol, 1 eq) in DMF (1 mL) was added CDI (49.35 mg, 304.36 μmol, 1.2 eq). Then the mixture was stirred at 30° C. for 0.5 h. and then 5-silaspiro[4.5]decan-8-amine (51.54 mg, 304.36 μmol, 1.2 eq) was added. The mixture was stirred at 30° C. for 11.5 h. LC-MS showed the starting material was consumed completely. The reaction mixture was added to water (20 mL), then filtered and the filter cake was washed with 5 mL of water, dried in vacuo to give product. The crude product diluted with EtOAc (10 ml) and concentrated under reduced pressure to give a residue. The residue was purified by column chromatography (SiO₂, Petroleum ether:EtOAc=1:0 to 10:1). The residue was diluted in ACN (1 mL) and H₂O (10 mL), then lyophilized. The product 4,6-difluoro-N-(5-silaspiro[4.5]decan-8-yl)-1H-indole-2-carboxamide (39 mg, 107.50 μmol, 42.39% yield, 96.052% purity) was obtained as a white solid.

LCMS (ESI) m/z 349.1 [M+H]⁺

¹H NMR (500 MHz, DMSO-d₆) δ=11.94 (br s, 1H), 8.30 (br d, J=8.2 Hz, 1H), 7.26 (s, 1H), 7.01 (br d, J=9.0 Hz, 1H), 6.87 (br t, J=10.2 Hz, 1H), 3.76 (br d, J=8.4 Hz, 1H), 2.06 (br d, J=10.2 Hz, 2H), 1.64-1.51 (m, 6H), 0.84-0.70 (m, 4H), 0.64-0.58 (m, 2H), 0.57-0.51 (m, 2H).

### Example 5, MPL-252

### Synthesis of 4,6-dichloro-N-(5-silaspiro[4.5]decan-8-yl)-1H-indole-2-carboxamide

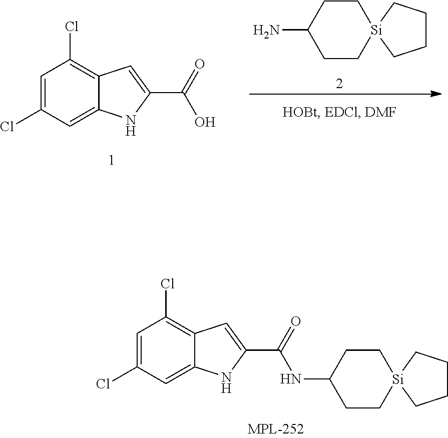

To a solution of 4,6-dichloro-1H-indole-2-carboxylic acid (0.4 g, 1.74 mmol, 1 eq) in DMF (10 mL) was added 5-silaspiro[4.5]decan-8-amine (357.84 mg, 1.74 mmol, 1 eq, HCl), HOBt (352.41 mg, 2.61 mmol, 1.5 eq), EDCI (499.98 mg, 2.61 mmol, 1.5 eq) and TEA (439.87 mg, 4.35 mmol, 605.04 uL, 2.5 eq). The mixture was stirred at 20° C. for 0.5 hr. TLC showed the reaction was complete. The mixture was poured into water (30 mL) and stirred for 5 min. The mixture was filtered. The filter cake was diluted with ACN (15 mL) and stirred for 10 min. The mixture was filtered again. The filter cake was dried under reduced pressure to give the product. Compound 4,6-dichloro-N-(5-silaspiro[4.5]decan-8-yl)-1H-indole-2-carboxamide (455 mg, 1.14 mmol, 65.60% yield, 95.6% purity) was obtained as a beige solid.

LCMS (ESI) m/z 381.0 [M+H]⁺

¹H NMR (400 MHz, DMSO-d₆) δ=7.41 (s, 1H), 7.20 (s, 1H), 7.10 (d, J=1.5 Hz, 1H), 3.81 (br t, J=11.4 Hz, 1H), 2.24-2.14 (m, 2H), 1.71-1.58 (m, 6H), 0.87-0.77 (m, 4H), 0.68 (t, J=6.9 Hz, 2H), 0.58 (br t, J=6.9 Hz, 2H).

### Example 6, MPL-262

### Synthesis of N-(1,1-dimethylsilepan-4-yl)-4,6-dimethyl-1H-indole-2-carboxamide

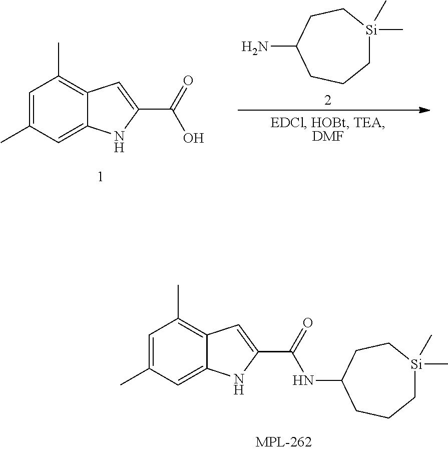

To a solution of 4,6-dimethyl-1H-indole-2-carboxylic acid (30 mg, 158.55 μmol, 1 eq) in DMF (0.5 mL) was added 1,1-dimethylsilepan-4-amine (50 mg, 317.81 μmol, 2 eq). A mixture of HOBt (32.14 mg, 237.83 μmol, 1.5 eq) and EDCI (45.59 mg, 237.83 μmol, 1.5 eq) in DMF (0.5 mL) was added followed by TEA (48.13 mg, 475.66 μmol, 66.21 μL, 3 eq). The mixture was stirred at 25° C. for 2 hr. LCMS showed there were no starting material and there was a main desired compound. The reaction was diluted in ACN (3 mL) and purified by pre. HPLC (column: YMC-Actus Triart C18 150*30 mm*5 um; mobile phase: [water (0.225% FA)-ACN]; B %: 67%-92%, 11 min). Compound N-(1,1-dimethylsilepan-4-yl)-4,6-dimethyl-1H-indole-2-carboxamide (30 mg, 91.14 μmol, 57.49% yield, 99.81% purity) was obtained as a white solid.

LCMS (ESI) m/z 329.1 [M+H]⁺

¹H NMR (500 MHz, CHLOROFORM-d) δ=9.14 (br s, 1H), 7.06 (s, 1H), 6.78 (br d, J=8.6 Hz, 2H), 6.09 (br d, J=6.7 Hz, 1H), 4.09 (br d, J=8.6 Hz, 1H), 2.52 (s, 3H), 2.43 (s, 3H), 2.15-2.01 (m, 2H), 1.85 (br d, J=8.6 Hz, 1H), 1.76-1.66 (m, 1H), 1.59-1.46 (m, 2H), 0.83-0.65 (m, 4H), 0.06 (d, J=2.3 Hz, 6H).

### Example 7, MPL-264

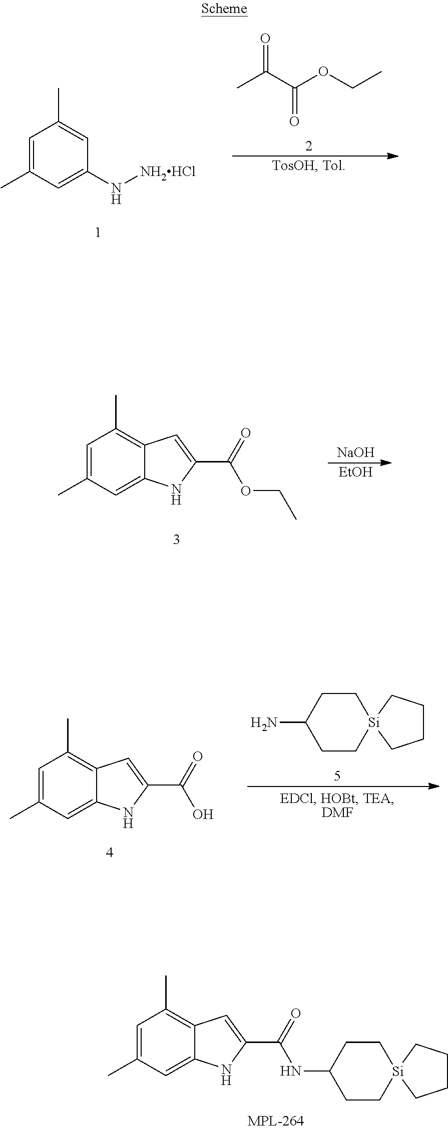

### Step 1. Synthesis of ethyl 4,6-dimethyl-1H-indole-2-carboxylate

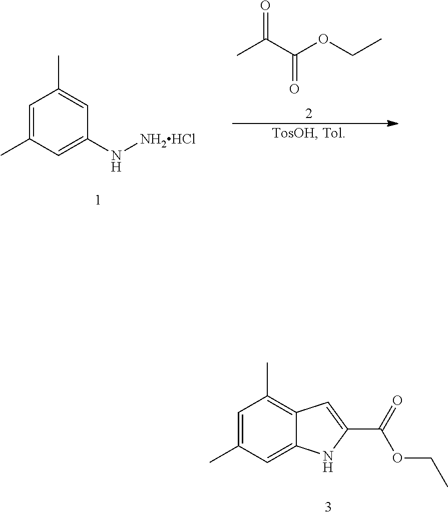

In a flask fitted with a Dean-Stark trap, (3,5-dimethylphenyl)hydrazine (15 g, 86.88 mmol, 1 eq, HCl), ethyl 2-oxopropanoate (10.09 g, 86.88 mmol, 9.61 mL, 1 eq) and TosOH (29.92 g, 173.76 mmol, 2 eq) in Tol. (150 mL) was heated to reflux at 140° C. for 12 hr. TLC (Petroleum ether:EtOAc=1:1, R_{f}=0.8) showed there were no starting material and one major new spot with lower polarity. The mixture was concentrated in reduced pressure. The crude product was purified by silica gel column chromatography (eluent of 0˜20% EtOAc: Petroleum ether gradient, 80 g silica gel column). All fractions found to contain the product by TLC (Petroleum ether:EtOAc=5:1, Rf=0.3) were combined and evaporated. Compound ethyl 4,6-dimethyl-1H-indole-2-carboxylate (8 g, 34.98 mmol, 40.26% yield, 95% purity) was obtained as a white solid which was confirmed by TLC.

### Step 2. Synthesis of 4,6-dimethyl-1H-indole-2-carboxylic acid

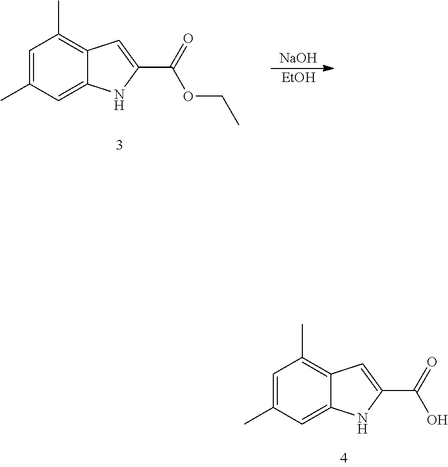 [SMILES: "EtOH" ; CCOC(=O)c1cc2c(C)cc(C)cc2[nH]1 ; "NaOH" ; Cc1cc(C)c2cc(C(=O)O)[nH]c2c1]

To a solution of ethyl 4,6-dimethyl-1H-indole-2-carboxylate (8 g, 36.82 mmol, 1 eq) in EtOH (40 mL) and H₂O (40 mL) was added LiOH.H₂O (4.64 g, 110.47 mmol, 3 eq). The mixture was stirred at 50° C. for 12 hr. TLC (Petroleum ether:EtOAc=3:1) showed there were no starting material and one major new spot with higher polarity was detected. The mixture was concentrated in reduced pressure. The residue was diluted with H₂O (50 mL). The mixture was adjusted to pH=4 with 1N HCl. There was much precipitation which was collected by filter. The cake was diluted in EtOAc (30 mL), dried with anhydrous MgSO₄, filtered. The filtrate was concentrated in vacuo. The residue was used directly for next step without further purification. Compound 4,6-dimethyl-1H-indole-2-carboxylic acid (5.9 g, 29.62 mmol, 80.45% yield, 95% purity) was obtained as a white solid which was confirmed by LCMS and ¹HNMR.

LCMS (ESI) m/z 190.1 [M+H]⁺

### Step 3. Synthesis of 4,6-dimethyl-N-(5-silaspiro[4.5]decan-8-yl)-1H-indole-2-carboxamide

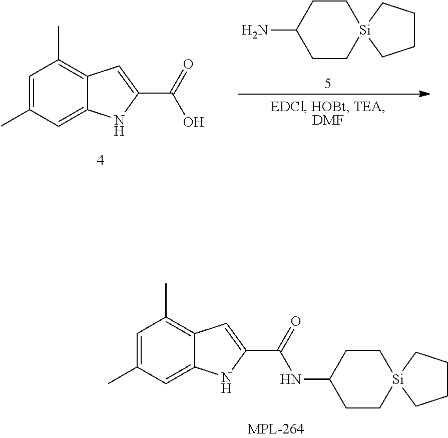

To a solution of 4,6-dimethyl-1H-indole-2-carboxylic acid (30 mg, 158.55 μmol, 1 eq) in DMF (0.5 mL) was added 5-silaspiro[4.5]decan-8-amine (35.89 mg, 174.41 μmol, 1.1 eq, HCl salt). A mixture of EDCI (45.59 mg, 237.83 μmol, 1.5 eq) and HOBt (32.14 mg, 237.83 μmol, 1.5 eq) in DMF (0.5 mL) was added, followed by TEA (48.13 mg, 475.66 μmol, 66.21 uL, 3 eq). The mixture was stirred at 30° C. for 2 hr. LCMS showed there were no starting material and one major product was detected. The reaction was added dropwise to H₂O (20 mL). There was much precipitation which was collected by filter. The cake was washed with H₂O (20 mL), then dried under reduced pressure. The residue was delivered without further purification. Compound 4,6-dimethyl-N-(5-silaspiro[4.5]decan-8-yl)-1H-indole-2-carboxamide (49.7 mg, 141.55 μmol, 89.28% yield, 96.99% purity) was obtained as a white solid which was confirmed by LCMS and ¹HNMR.

LCMS (ESI) m/z 341.1 [M+H]⁺

¹H NMR (500 MHz, CHLOROFORM-d) δ=9.04 (br s, 1H), 7.06 (s, 1H), 6.79 (s, 1H), 6.78-6.75 (m, 1H), 6.77 (s, 1H), 6.05-5.98 (m, 1H), 3.96 (br d, J=7.8 Hz, 1H), 2.52 (s, 3H), 2.43 (s, 3H), 2.26 (br d, J=11.0 Hz, 2H), 1.67-1.60 (m, 6H), 0.87-0.80 (m, 4H), 0.66-0.56 (m, 4H).

### Example 8, MPL-265

### Synthesis of 4,6-dimethyl-N-(6-silaspiro[5.5]undecan-3-yl)-1H-indole-2-carboxamide

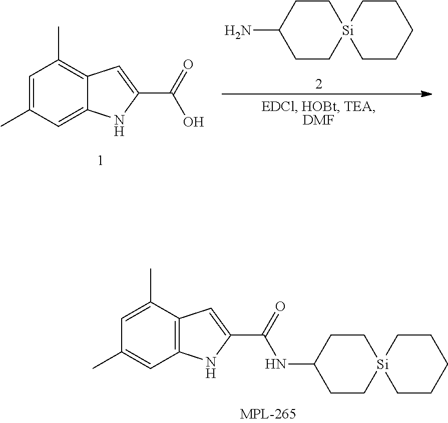

To a solution of 4,6-dimethyl-1H-indole-2-carboxylic acid (30 mg, 158.55 μmol, 1 eq) in DMF (0.5 mL) was added 6-silaspiro[5.5]undecan-3-amine (38.34 mg, 174.41 μmol, 1.1 eq, HCl salt). A mixture of EDCI (45.59 mg, 237.83 μmol, 1.5 eq) and HOBt (32.14 mg, 237.83 μmol, 1.5 eq) in DMF (0.5 mL) followed by TEA (48.13 mg, 475.66 μmol, 66.21 uL, 3 eq). The mixture was stirred at 30° C. for 2 hr. LCMS showed there were no starting material and main desired compound. The reaction was added dropwise to H₂O (20 mL). There was much precipitation which was collected by filter. The cake was washed with H₂O (20 mL), then concentrated under reduced pressure. The residue was delivered without further purification. Compound 4,6-dimethyl-N-(6-silaspiro[5.5] undecan-3-yl)-1H-indole-2-carboxamide (57.2 mg, 154.99 μmol, 97.75% yield, 96.07% purity) was obtained as a white solid which was confirmed by LCMS and ¹HNMR.

LCMS (ESI) m/z 355.1 [M+H]⁺

¹H NMR (500 MHz, CHLOROFORM-d) δ=8.96 (br s, 1H), 7.06 (s, 1H), 6.80-6.77 (m, 1H), 6.76 (d, J=1.4 Hz, 1H), 6.00 (br d, J=8.4 Hz, 1H), 3.96-3.89 (m, 1H), 2.52 (s, 3H), 2.43 (s, 3H), 2.22-2.16 (m, 2H), 1.73-1.64 (m, 4H), 1.63-1.57 (m, 2H), 1.46-1.39 (m, 2H), 0.91 (br d, J=14.6 Hz, 2H), 0.74-0.67 (m, 4H), 0.65-0.61 (m, 2H).

### Example 9, MPL-286

### Synthesis of N-(1,1-dimethylsilepan-4-yl)-4,6-difluoro-1H-indole-2-carboxamide

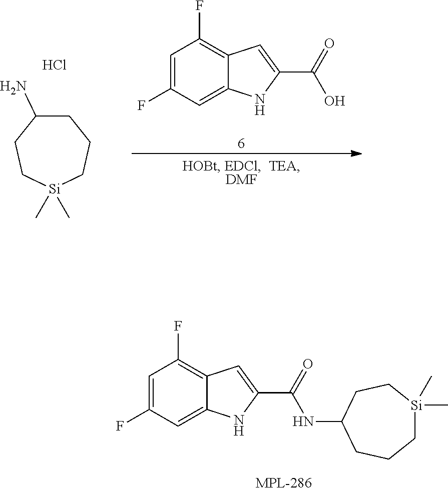

A solution of EDCI (72.93 mg, 380.44 μmol, 1.5 eq) and HOBt (51.41 mg, 380.44 μmol, 1.5 eq) in DMF (0.5 mL) was added to a solution of 4,6-difluoro-1H-indole-2-carboxylic acid (50 mg, 253.63 μmol, 1 eq) and 1,1-dimethylsilepan-4-amine (98.30 mg, 507.26 μmol, 2 eq, HCl) in DMF (0.5 mL) with stirring. Then TEA (76.99 mg, 760.89 μmol, 105.91 μL, 3 eq) was added to above solution. The mixture was stirred at 25° C. for 2 hr. LCMS showed the desired product was detected. The mixture was purified by prep-HPLC (column: YMC-Actus Triart C18 150*30 mm*5 um; mobile phase: [water (0.225% FA)-ACN]; B %: 65%-92%, 11 min). Compound N-(1,1-dimethylsilepan-4-yl)-4,6-difluoro-1H-indole-2-carboxamide (31 mg, 91.29 μmol, 35.99% yield, 99.082% purity) was obtained as a white solid.

LCMS (ESI), m/z 337.1[M+H]⁺

1H NMR (500 MHz, DMSO-d6) δ=11.88 (br s, 1H), 8.27 (br d, J=8.1 Hz, 1H), 7.23 (s, 1H), 6.97 (br d, J=9.3 Hz, 1H), 6.88-6.75 (m, 1H), 3.91-3.80 (m, 1H), 1.95-1.71 (m, 3H), 1.69-1.57 (m, 1H), 1.52-1.38 (m, 2H), 0.77-0.64 (m, 2H), 0.62-0.47 (m, 2H), 0.00 (d, J=11.1 Hz, 6H).

### Example 10, MPL-296, MPL-296A and MPL-296B

### Synthesis of N-(1,1-dimethylsilolan-3-yl)-4,6-dimethyl-1H-indole-2-carboxamide, N-[(3R)-1,1-dimethylsilolan-3-yl]-4,6-dimethyl-1H-indole-2-carboxamide; and N-[(3S)-1,1-dimethylsilolan-3-yl]-4,6-dimethyl-1H-indole-2-carboxamide

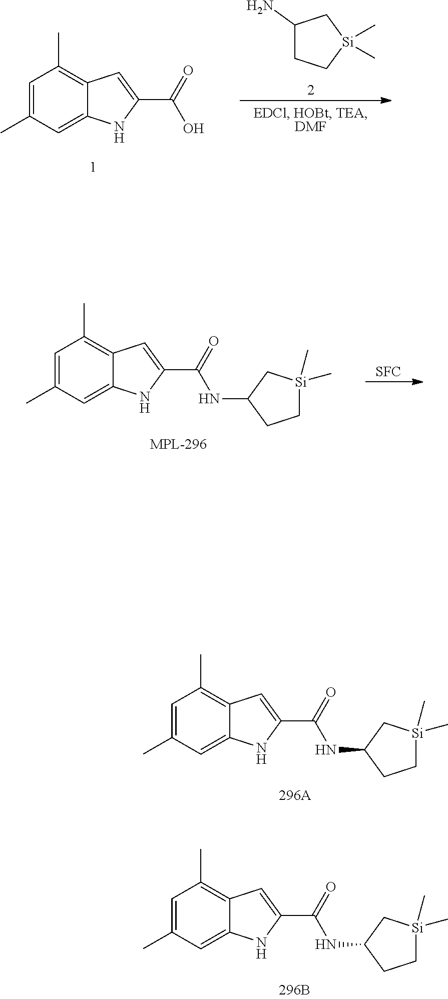

To a solution of 4,6-dimethyl-1H-indole-2-carboxylic acid (30 mg, 158.55 umol, 1 eq) and 1,1-dimethylsilolan-3-amine (26.28 mg, 158.55 umol, 1 eq, HCl) in DMF (1.5 mL) was added a solution of EDCI (91.18 mg, 475.66 umol, 3 eq) and HOBt (64.27 mg, 475.66 umol, 3 eq) in DMF (1 mL), followed by TEA (48.13 mg, 475.66 umol, 66.21 uL, 3 eq). The mixture was stirred at 25° C. for 1 hr. LC-MS showed desired product. The mixture was filtered and the filtrate was purified by prep-HPLC (YMC-Actus Triart C18 150*30 mm*5 um; mobile phase: A: 0.225% formic acid in water, B: CH₃CN; gradient: 60%-90% B over 11 min). Compound N-(1,1-dimethylsilolan-3-yl)-4,6-dimethyl-1H-indole-2-carboxamide (28.8 mg, 95.39 umol, 60.16% yield, 99.52% purity) was obtained as a white solid.

LCMS (ESI) m/z: 301.1 [M+H]⁺

¹H NMR (500 MHz, CHLOROFORM-d) δ=9.16 (br s, 1H), 7.11-7.00 (m, 1H), 6.82-6.71 (m, 2H), 6.07 (br d, J=7.5 Hz, 1H), 4.23 (dq, J=6.9, 11.3 Hz, 1H), 2.55-2.47 (m, 3H), 2.46-2.39 (m, 3H), 2.29 (dddd, J=2.4, 5.3, 7.5, 10.0 Hz, 1H), 1.46-1.30 (m, 2H), 0.86 (ddd, J=2.4, 7.2, 15.0 Hz, 1H), 0.66 (ddd, J=8.0, 11.8, 15.0 Hz, 1H), 0.54 (dd, J=10.9, 14.1 Hz, 1H), 0.21 (d, J=2.0 Hz, 6H).

The reaction was also conducted at 528.5 umol. The product (MPL-296) from prep-HPLC purification was separated by prep-SFC (Waters Prep SFC 80Q, column: DAICEL CHIRALPAK AD (250 mm*30 mm, 10 um); mobile phase: A: 0.1% NH₃H₂O in EtOH; B CO₂, gradient: isocratic 15% B; flow rate: 60 mL/min). Two peaks (two enantiomers), N-[(3R)-1,1-dimethylsilolan-3-yl]-4,6-dimethyl-1H-indole-2-carboxamide and N-[(3S)-1,1-dimethylsilolan-3-yl]-4,6-dimethyl-1H-indole-2-carboxamide were obtained.

Peak 1 (MPL-296A): 38.5 mg, 127.05 umol, 24.04% yield, 99.158% purity, white solid.

LCMS m/z: 301.0 [M+H]⁺

¹H NMR (500 MHz, DMSO-d₆) δ=11.30 (s, 1H), 8.18 (d, J=7.8 Hz, 1H), 7.12 (d, J=1.4 Hz, 1H), 7.01 (s, 1H), 6.65 (s, 1H), 4.03 (dq, J=6.8, 11.7 Hz, 1H), 2.43 (s, 3H), 2.36-2.29 (m, 3H), 2.08-1.98 (m, 1H), 1.43 (dq, J=7.2, 12.2 Hz, 1H), 1.10 (ddd, J=1.8, 6.7, 14.2 Hz, 1H), 0.86-0.76 (m, 1H), 0.65 (dd, J=11.2, 14.1 Hz, 1H), 0.53 (ddd, J=8.0, 12.7, 14.5 Hz, 1H), 0.23-0.12 (m, 6H).

Peak 2 (MPL-296B): 38.2 mg, 127.13 umol, 24.06% yield, 100% purity, white solid.

LCMS m/z: 301.0 [M+H]⁺

¹H NMR (400 MHz, DMSO-d₆) δ=11.30 (br s, 1H), 8.18 (br d, J=7.8 Hz, 1H), 7.11 (d, J=1.2 Hz, 1H), 7.01 (s, 1H), 6.65 (s, 1H), 4.03 (br dd, J=7.2, 11.4 Hz, 1H), 2.43 (s, 3H), 2.33 (s, 3H), 2.02 (br d, J=5.1 Hz, 1H), 1.43 (dq, J=7.5, 12.1 Hz, 1H), 1.16-1.05 (m, 1H), 0.80 (br dd, J=6.0, 14.5 Hz, 1H), 0.65 (dd, J=11.4, 14.1 Hz, 1H), 0.59-0.46 (m, 1H), 0.18 (s, 6H).

MPL-296A and MPL-296B were also analyzed by analytical SFC.

Conditions:

Instrument: Waters UPCC with PDA Detector

Column: Chiralcel OJ-3 150 mm×4.6 mm, 3 um particle size

Mobile phase: A: CO₂, B: 0.05% DEA in ethanol

Gradient: 5% to 40% B in 5 min, 40% to 5% B in 0.5 min, hold 5% B for 1.5 min

Flow rate: 2.5 mL/min

Column temp: 35° C.

ABPR: 1500 psi

MPL-296A: retention time 2.84 min, 99.8% ee

MPL-296B: retention time 2.98 min, 87.7% ee

### Example 11, MPL-303

### Synthesis of N-(1,1-dimethylsilocan-5-yl)-4,6-dimethyl-1H-indole-2-carboxamide

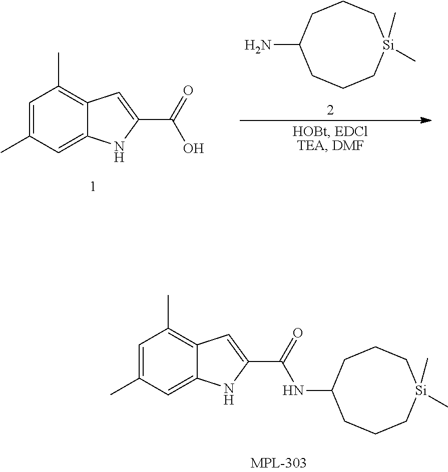

To a solution of 4,6-dimethyl-1H-indole-2-carboxylic acid (32.78 mg, 173.23 umol, 1.2 eq) and 1,1-dimethylsilocan-5-amine (30 mg, 144.36 umol, 1 eq, HCl) in DMF (1.5 mL) was added a solution of EDCI (83.02 mg, 433.08 umol, 3 eq) and HOBt (58.52 mg, 433.08 umol, 3 eq) in DMF (1 mL), followed by TEA (87.65 mg, 866.15 umol, 120.56 uL, 6 eq). The mixture was stirred at 20° C. for 1 hr. LC-MS indicated desired mass was detected. The reaction mixture was filtered. The filtrate was purified by prep-HPLC (YMC-Actus Triart C18 150*30 mm*5 um; mobile phase: A: 0.225% formic acid in water, B: CH₃CN; gradient: 75%-100% B over 11 min). The product was further purified by prep-SFC (Berger MG II, column: DAICEL CHIRALPAK AS (250 mm*30 mm, 10 um); mobile phase: A: 0.1% NH₃H₂O in EtOH; B: CO₂, gradient: 25% B, isocratic, flow rate: 60 mL/min). Compound N-(1,1-dimethylsilocan-5-yl)-4,6-dimethyl-1H-indole-2-carboxamide (13.3 mg, 38.83 umol, 26.90% yield, 100% purity) was obtained as a white solid.

LCMS m/z: 343.1 [M+1]⁺

¹H NMR (500 MHz, DMSO-d₆) δ=11.31 (s, 1H), 8.20 (d, J=8.2 Hz, 1H), 7.12 (s, 1H), 7.01 (s, 1H), 6.65 (s, 1H), 4.04 (br d, J=5.8 Hz, 1H), 2.45 (br s, 1H), 2.43 (s, 3H), 2.33 (s, 3H), 1.77-1.51 (m, 8H), 0.81-0.64 (m, 4H), 0.08-−0.04 (m, 6H).

### Example 12, MPL-317

### Synthesis of N-(1,1-dimethylsilinan-4-yl)-4,6-dimethyl-1H-indole-2-carboxamide

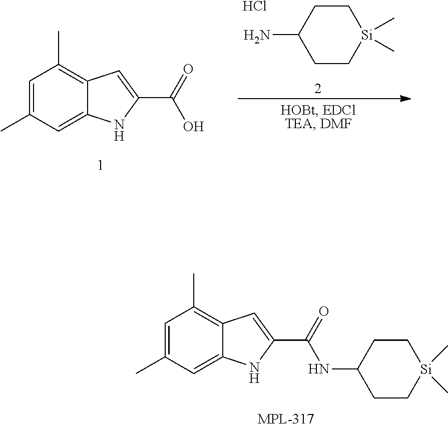

To a solution of 4,6-dimethyl-1H-indole-2-carboxylic acid (2.30 g, 12.13 mmol, 1 eq) and 1,1-dimethylsilinan-4-amine (2.40 g, 13.34 mmol, 1.1 eq, HCl salt) in DMF (10 mL), a solution of HOBt (4.92 g, 36.39 mmol, 3 eq) and EDCI (6.98 g, 36.39 mmol, 3 eq) in DMF (20 mL) was added with stirring, followed by TEA (6.14 g, 60.65 mmol, 8.44 mL, 5 eq). The reaction mixture was stirred at 25° C. for 2 hr. LCMS showed starting material was consumed completely. The residue was poured into NaHCO₃ solution (saturated NaHCO₃: H₂O=2:1, 270 mL), precipitates formed. The mixture was stirred at 25° C. for 10 min and then filtered. The cake was washed with water (20 mL×5) and collected, and then triturated with water (50 mL) at 25° C. for 30 min and filtered. The collected cake was washed with water (20 mL×3) and then triturated with CH₃CN (50 mL) at 25° C. for 30 min, and then filtered. The cake was washed with CH₃CN (20 mL×3) and collected. Compound N-(1,1-dimethylsilinan-4-yl)-4,6-dimethyl-1H-indole-2-carboxamide (3.960 g, 12.56 mmol, 86.31% yield, 99.78% purity) was obtained as a white solid.

LCMS (ESI) m/z 315.2 [M+H]⁺

¹H NMR (500 MHz, DMSO-d₆) δ=11.30 (s, 1H), 8.12 (d, J=8.2 Hz, 1H), 7.11 (s, 1H), 7.01 (s, 1H), 6.65 (s, 1H), 3.70 (dt, J=8.2, 11.2 Hz, 1H), 2.43 (s, 3H), 2.37-2.30 (m, 3H), 1.98 (br d, J=9.6 Hz, 2H), 1.65-1.52 (m, 2H), 0.77 (br d, J=14.5 Hz, 2H), 0.61 (dt, J=4.7, 14.2 Hz, 2H), 0.09 (s, 3H), 0.06-0.00 (m, 3H).

### Example 13, MPL-324

### Synthesis of N-(1,1-dimethylsilepan-4-yl)-4,6-bis(trifluoromethyl)-1H-indole-2-carboxamide

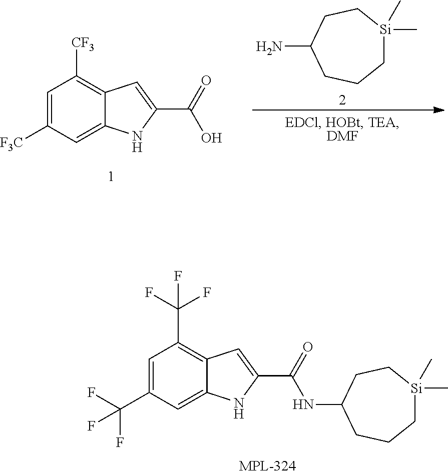

To a solution of 4,6-bis(trifluoromethyl)-1H-indole-2-carboxylic acid (30 mg, 100.96 umol, 1 eq) and 1,1-dimethylsilepan-4-amine (23.48 mg, 121.15 umol, 1.2 eq, HCl salt) in DMF (0.5 mL) was added a solution of EDCI (58.06 mg, 302.88 umol, 3 eq) and HOBt (40.93 mg, 302.88 umol, 3 eq) in DMF (0.5 mL), followed by TEA (51.08 mg, 504.80 umol, 70.26 uL, 5 eq). The mixture was stirred at 30° C. for 1 hr. LCMS showed the desired product was detected. The mixture was purified by prep-HPLC (YMC-Actus Triart C18 150*30 mm*5 um; mobile phase: A: 0.225% formic acid in water, B: CH₃CN, gradient 75%-100% B over 11 min). Compound N-(1,1-dimethylsilepan-4-yl)-4,6-bis(trifluoromethyl)-1H-indole-2-carboxamide (20.7 mg, 47.43 umol, 46.98% yield, 100% purity) was obtained as a white solid.

LCMS (ESI), m/z 437.1[M+H]⁺

¹H NMR (500 MHz, DMSO-d₆) δ=ppm 12.53 (br s, 1H) 8.63 (d, J=7.93 Hz, 1H) 7.97 (s, 1H) 7.61 (s, 1H) 7.47 (s, 1H) 3.79-3.95 (m, 1H) 1.72-1.93 (m, 3H) 1.58-1.69 (m, 1H) 1.36-1.51 (m, 2H) 0.65-0.79 (m, 2H) 0.52-0.62 (m, 2H)-0.01 (d, J=12.66 Hz, 6H).

### Example 14, MPL-325

### Synthesis of N-(1,1-dimethylsilocan-4-yl)-4,6-bis(trifluoromethyl)-1H-indole-2-carboxamide

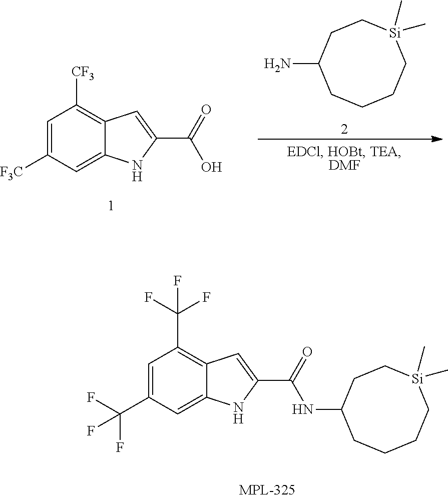

To a solution of 4,6-bis(trifluoromethyl)-1H-indole-2-carboxylic acid (30 mg, 100.96 umol, 1 eq) and 1,1-dimethylsilocan-4-amine (20.98 mg, 100.96 umol, 1 eq, HCl salt) in DMF (0.5 mL) was added a solution of EDCI (58.06 mg, 302.88 umol, 3 eq) and HOBt (40.93 mg, 302.88 umol, 3 eq) in DMF (0.5 mL), followed by TEA (51.08 mg, 504.80 umol, 70.26 uL, 5 eq). The mixture was stirred at 30° C. for 1 hr. LCMS showed the desired product was detected. The mixture was purified by prep-HPLC (column: YMC-Actus Triart C18 150*30 mm*5 um; mobile phase: A: 0.225% formic acid in water, B: CH3CN, gradient 75%-100% B over 11 min). Compound N-(1,1-dimethylsilocan-4-yl)-4,6-bis(trifluoromethyl)-1H-indole-2-carboxamide (21.8 mg, 47.72 umol, 47.27% yield, 98.615% purity) was obtained as a white solid.

LCMS (ESI), m/z 451.0 [M+H]⁺; ¹H NMR (500 MHz, DMSO-d₆) δ=12.58 (s, 1H) 8.61 (d, J=7.93 Hz, 1H) 8.01 (s, 1H) 7.65 (s, 1H) 7.53 (s, 1H) 3.90-4.12 (m, 1H) 1.31-1.94 (m, 8H) 0.46-0.89 (m, 4H) −0.12-0.16 (m, 6H).

### Example 15, MPL-326

### Synthesis of N-(1,1-dimethylsilocan-5-yl)-4,6-bis(trifluoromethyl)-1H-indole-2-carboxamide

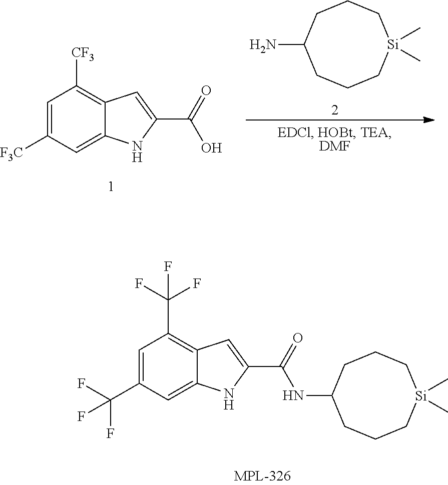

To a solution of 4,6-bis(trifluoromethyl)-1H-indole-2-carboxylic acid (50 mg, 168.26 umol, 1 eq) and 1,1-dimethylsilocan-5-amine (28.83 mg, 168.26 umol, 1 eq, HCl salt) in DMF (1 mL) was added a solution of EDCI (96.77 mg, 504.79 umol, 3 eq) and HOBt (68.21 mg, 504.79 umol, 3 eq) in DMF (1 mL), followed by TEA (85.13 mg, 841.32 umol, 117.10 uL, 5 eq). The mixture was stirred at 30° C. for 1 hr. LCMS showed the desired product was detected. The mixture was purified by prep-HPLC (column: YMC-Actus Triart C18 150*30 mm*5 um; mobile phase: A: 0.225% formic acid in water, B: CH₃CN, gradient: 78%-100% B over 11 min). The product from prep-HPLC was further purified by prep-SFC (Sepiatec Prep SFC 100, column: DAICEL CHIRALPAK AD-H (250 mm*30 mm, 5 um); mobile phase: A: 0.1% NH₃H₂O in IPA; B: CO₂, gradient: 15% B, isocratic). Compound N-(1,1-dimethylsilocan-5-yl)-4,6-bis(trifluoromethyl)-1H-indole-2-carboxamide (13.4 mg, 29.35 umol, 17.44% yield, 98.656% purity) was obtained as a white solid.

LCMS (ESI), m/z 451.1 [M+H]⁺; ¹H NMR (400 MHz, DMSO-d6)) δ=0.24-0.23 (m, 6H) 0.58-0.91 (m, 4H) 1.65 (br s, 8H) 4.07 (br s, 1H) 7.50 (br s, 1H) 7.65 (s, 1H) 8.02 (s, 1H) 8.71 (br d, J=7.82 Hz, 1H) 12.58 (br s, 1H).

### Example 16, MPL-327

### Synthesis of N-(1,1-dimethylsilolan-3-yl)-4,6-bis(trifluoromethyl)-1H-indole-2-carboxamide

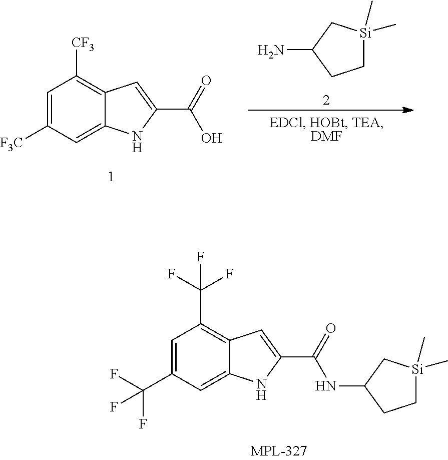

To a solution of 4,6-bis(trifluoromethyl)-1H-indole-2-carboxylic acid (30 mg, 100.96 umol, 1 eq) and 1,1-dimethylsilolan-3-amine (16.73 mg, 100.96 umol, 1 eq, HCl salt) in DMF (1 mL) was added a solution of EDCI (58.06 mg, 302.87 umol, 3 eq) and HOBt (40.92 mg, 302.87 umol, 3 eq) in DMF (1 mL), followed by TEA (51.08 mg, 504.79 umol, 70.26 uL, 5 eq). The mixture was stirred at 30° C. for 1 hr. LCMS showed the desired product was detected. The mixture was purified by prep-HPLC (column: YMC-Actus Triart C18 150*30 mm*5 um; mobile phase: A: 0.225% formic acid in water, B: CH₃CN, gradient: 66%-94% B over 11 min). Compound N-(1,1-dimethylsilolan-3-yl)-4,6-bis(trifluoromethyl)-1H-indole-2-carboxamide (17.1 mg, 41.79 umol, 41.39% yield, 99.81% purity) was obtained as a white solid.

LCMS (ESI), m/z 409.0 [M+H]⁺

¹H NMR (500 MHz, DMSO-d₆) δ=12.43 (br s, 1H) 8.54 (br d, J=7.32 Hz, 1H) 7.85 (br s, 1H) 7.49 (br s, 1H) 7.33 (br s, 1H) 3.89 (br d, J=5.19 Hz, 1H) 1.89 (br s, 1H) 1.20-1.35 (m, 1H) 0.96 (br dd, J=13.66, 6.03 Hz, 1H) 0.64 (br dd, J=14.19, 6.56 Hz, 1H) 0.49 (br t, J=12.74 Hz, 1H) 0.30-0.41 (m, 1H) 0.00 (s, 6H).

### Example 17, MPL-334

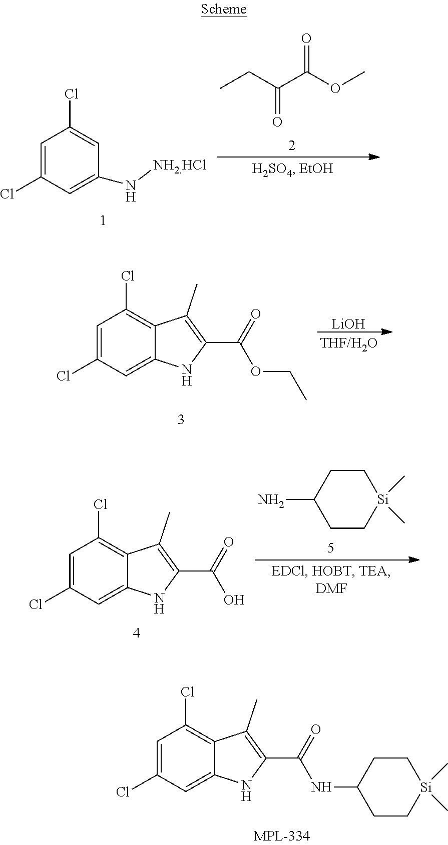

### Step 1. Synthesis of ethyl 4,6-dichloro-3-methyl-1H-indole-2-carboxylate

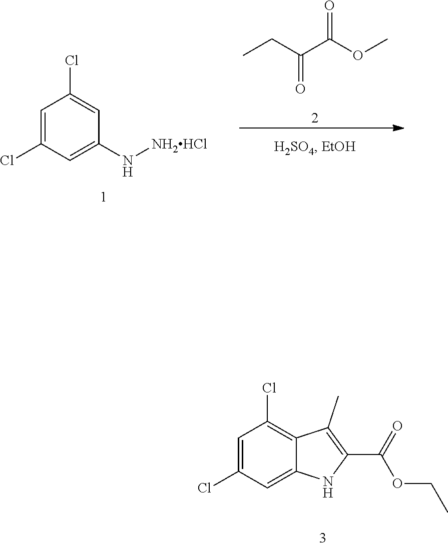

A solution of (3,5-dichlorophenyl)hydrazine (1 g, 4.68 mmol, 1 eq, HCl salt) and methyl 2-oxobutanoate (543.88 mg, 4.68 mmol, 1 eq) in EtOH (5 mL) was evaporated under reduced pressure. EtOH (4.5 mL) and H₂SO₄ (12 M, 0.5 mL, 98% purity, 1.28 eq) were added and the mixture was stirred at 80° C. for 24 hr. LCMS showed the starting material was consumed completely. The reaction was cooled to room temperature and partitioned between ethyl acetate (100 mL) and water (200 mL). The aqueous phase was extracted with ethyl acetate (100 mL). The combined organic layer was washed with saturated sodium bicarbonate solution, filtered and concentrated in vacuo. Ethyl 4,6-dichloro-3-methyl-1H-indole-2-carboxylate (1.2 g, crude) was obtained as a brown solid, which was used in the next step without further purification. ¹H NMR was recorded.

### Step 2. Synthesis of 4,6-dichloro-3-methyl-1H-indole-2-carboxylic acid

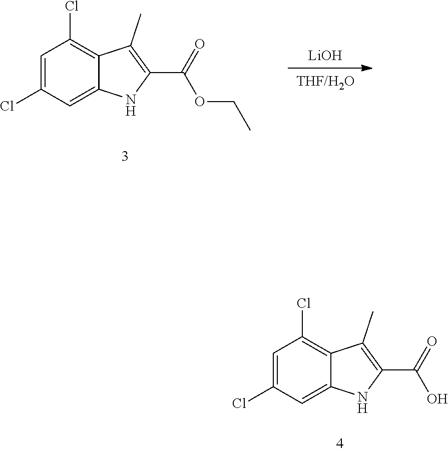 [SMILES: CCOC(=O)c1[nH]c2cc(Cl)cc(Cl)c2c1C ; "LiOH" ; "THF/H2O" ; Cc1c(C(=O)O)[nH]c2cc(Cl)cc(Cl)c12]

To a solution of ethyl 4,6-dichloro-3-methyl-1H-indole-2-carboxylate (1.2 g, 4.41 mmol, 1 eq) in THE (12 mL) and H₂O (12 mL) was added LiOH.H₂O (740.19 mg, 17.64 mmol, 4 eq). The reaction mixture was stirred at 50° C. for 12 hr. LCMS showed the desired product was detected. The mixture was concentrated in vacuo, and resulting residue was adjusted to pH to 4 with aqueous HCl (6 M), and then filtered. The cake was washed with water (30 mL) at 25° C. for 30 min and filtered. The cake was collected and washed with petroleum ether (20 mL). 4,6-dichloro-3-methyl-1H-indole-2-carboxylic acid (740 mg, 2.88 mmol, 65.32% yield, 95% purity) was obtained as a yellow solid.

¹H NMR (400 MHz, DMSO-d6) δ=13.298 (br, s, 1H), 11.855 (s, 1H), 7.365 (s, 1H), 7.134 (s, 1H), 2.764 (s, 3H).

### Step 3. Synthesis of 4,6-dichloro-N-(1,1-dimethylsilinan-4-yl)-3-methyl-1H-indole-2-carboxamide

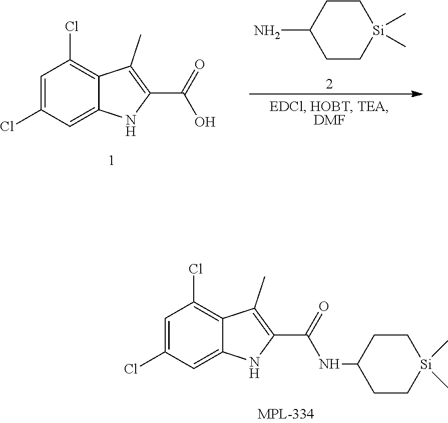

To a solution of 4,6-dichloro-3-methyl-1H-indole-2-carboxylic acid (100 mg, 409.71 umol, 1 eq) and 1,1-dimethylsilinan-4-amine (88.38 mg, 491.65 umol, 1.2 eq, HCl salt) in DMF (3 mL), a solution HOBt (83.04 mg, 614.57 umol, 1.5 eq) and EDCI (117.81 mg, 614.57 umol, 1.5 eq) in DMF (3 mL) was added, followed by TEA (207.29 mg, 2.05 mmol, 285.13 uL, 5 eq). The reaction mixture was stirred at 25° C. for 30 min. LCMS showed the desired product was detected. The reaction mixture was concentrated in vacuo. The residue was purified by prep-HPLC (column: YMC-Actus Triart C18 150*30 mm*5 um; mobile phase: A: 0.225% formic acid in water, B: CH₃CN, gradient 85%-100% B over 11 min). 4,6-dichloro-N-(1,1-dimethylsilinan-4-yl)-3-methyl-1H-indole-2-carboxamide (25.7 mg, 69.58 umol, 16.98% yield, 100% purity) was obtained as a white solid.

LCMS (ESI) m/z 369.1 [M+H]⁺

¹H NMR (400 MHz, DMSO-d₆) δ=11.64 (s, 1H), 7.94 (br d, J=7.8 Hz, 1H), 7.37 (d, J=1.7 Hz, 1H), 7.10 (d, J=1.7 Hz, 1H), 3.71 (dt, J=7.9, 10.7 Hz, 1H), 2.65 (s, 3H), 2.01 (br d, J=10.0 Hz, 2H), 1.66-1.56 (m, 2H), 0.78 (br d, J=14.7 Hz, 2H), 0.60 (dt, J=4.6, 13.8 Hz, 2H), 0.07 (s, 6H).

### Example 18, MPL-335

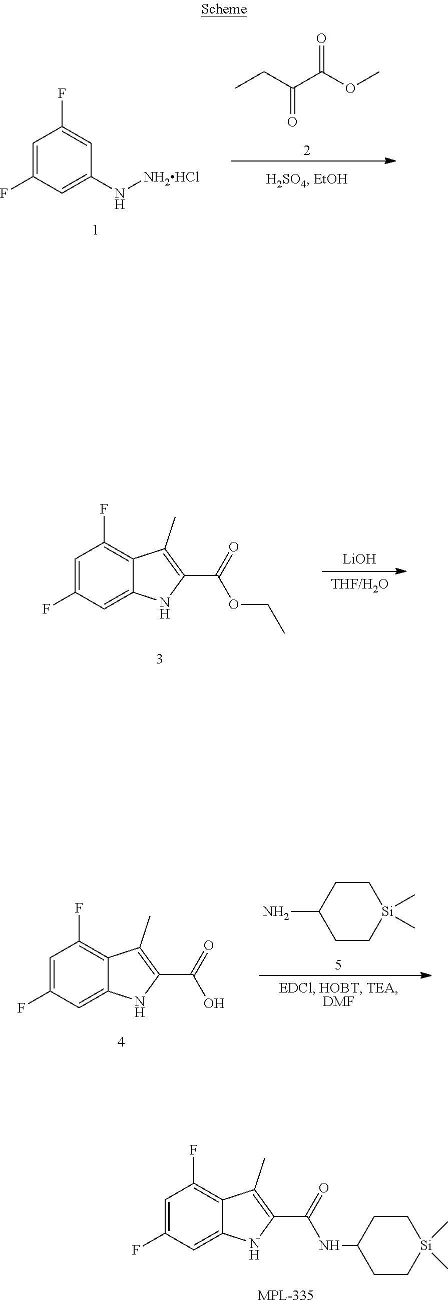

### Step 1. Synthesis of ethyl 4,6-difluoro-3-methyl-1H-indole-2-carboxylate

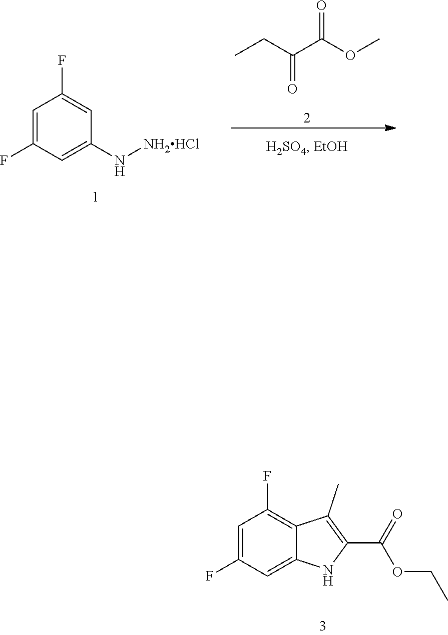

A solution of (3,5-difluorophenyl)hydrazine (1 g, 5.54 mmol, 1 eq, HCl salt) and methyl 2-oxobutanoate (643.00 mg, 5.54 mmol, 1 eq) in EtOH (2 mL) was evaporated under reduced pressure. EtOH (4.5 mL) and H₂SO₄ (12 M, 0.5 mL, 98% purity, 1.08 eq) were added and the mixture was stirred at 80° C. for 12 hr. LCMS showed the starting material was consumed completely. The residue was poured into water (15 mL) and stirred. The aqueous phase was extracted with ethyl acetate (10 mL×3). The combined organic phase was washed with brine (10 mL×2), dried with anhydrous Na₂SO₄, and filtered and concentrated in vacuo. The residue was purified by silica gel chromatography (Petroleum ether. Ethyl acetate=5:1). Ethyl 4,6-difluoro-3-methyl-1H-indole-2-carboxylate (680 mg, 2.79 mmol, 50.31% yield, 98% purity) was obtained as a white solid. ¹H NMR was recorded.

### Step 2. Synthesis of 4,6-difluoro-3-methyl-1H-indole-2-carboxylic acid

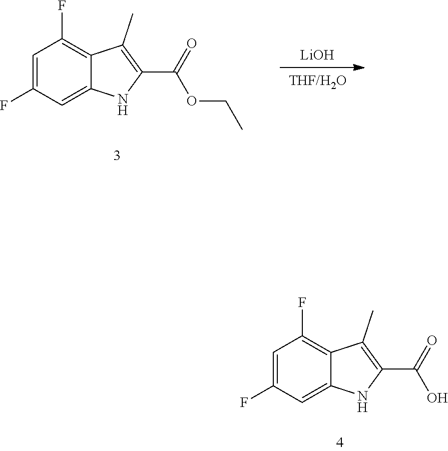 [SMILES: CCOC(=O)c1[nH]c2cc(F)cc(F)c2c1C ; "LiOH" ; "THF/H2O" ; Cc1c(C(=O)O)[nH]c2cc(F)cc(F)c12]

A solution of ethyl 4,6-difluoro-3-methyl-1H-indole-2-carboxylate (680 mg, 2.84 mmol, 1 eq) in THE (7 mL) and H₂O (7 mL) was added LiOH.H₂O (715.71 mg, 17.06 mmol, 6 eq). The reaction mixture was stirred at 50° C. for 12 hr. LCMS showed the starting material was consumed completely. The solution was concentrated in vacuo. Aqueous HCl (6 M) was added to the residue till pH to 4. The mixture was filtered, and cake was collected. The crude product was purified by re-crystallization from water (30 mL) at 25° C. for 30 min, and then further purified by re-crystallization from petroleum ether (20 mL) at 25° C. for 30 min. 4,6-difluoro-3-methyl-1H-indole-2-carboxylic acid (500 mg, 2.37 mmol, 83.30% yield, 100% purity) was obtained as a white solid. ¹H NMR was recorded.

### Step 3. Synthesis of N-(1,1-dimethylsilinan-4-yl)-4,6-difluoro-3-methyl-1H-indole-2-carboxamide

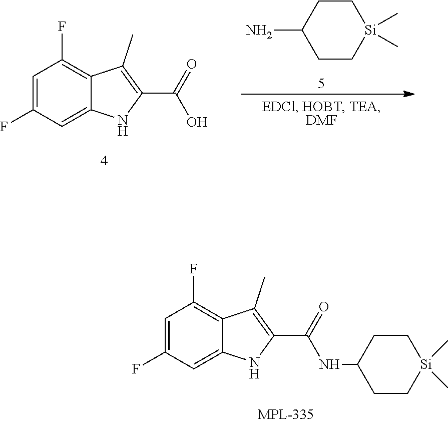

To a solution of 4,6-difluoro-3-methyl-1H-indole-2-carboxylic acid (100 mg, 473.56 umol, 1 eq) and 1,1-dimethylsilinan-4-amine (102.15 mg, 568.28 umol, 1.2 eq, HCl) in DMF (2 mL) was added a solution of HOBt (95.98 mg, 710.35 umol, 1.5 eq) and EDCI (136.17 mg, 710.35 umol, 1.5 eq) in DMF (3 mL) followed by TEA (239.60 mg, 2.37 mmol, 329.57 uL, 5 eq). The reaction mixture was stirred at 25° C. for 5 hr. LCMS showed the desired product was detected. The reaction mixture was concentrated in vacuo. The residue was purified by prep-HPLC (column: YMC-Actus Triart C18 150*30 mm*5 um; mobile phase: A: 0.225% formic acid in water, B: ACN; gradient: 70%-97% B over 11 min). N-(1,1-dimethylsilinan-4-yl)-4,6-difluoro-3-methyl-1H-indole-2-carboxamide (119.1 mg, 353.99 umol, 74.75% yield, 100% purity) was obtained as a white solid.

LCMS (ESI) m/z 337.1 [M+H]⁺

¹H NMR (400 MHz, DMSO-d6) δ=11.51 (s, 1H), 7.76 (br d, J=7.8 Hz, 1H), 7.00 (dd, J=2.0, 9.3 Hz, 1H), 6.83-6.75 (m, 1H), 3.74-3.66 (m, 1H), 2.56 (s, 3H), 2.01 (br d, J=10.3 Hz, 2H), 1.65-1.54 (m, 2H), 0.77 (br d, J=14.7 Hz, 2H), 0.60 (dt, J=4.8, 13.9 Hz, 2H), 0.06 (d, J=17.1 Hz, 6H).

### Example 19, MPL-336

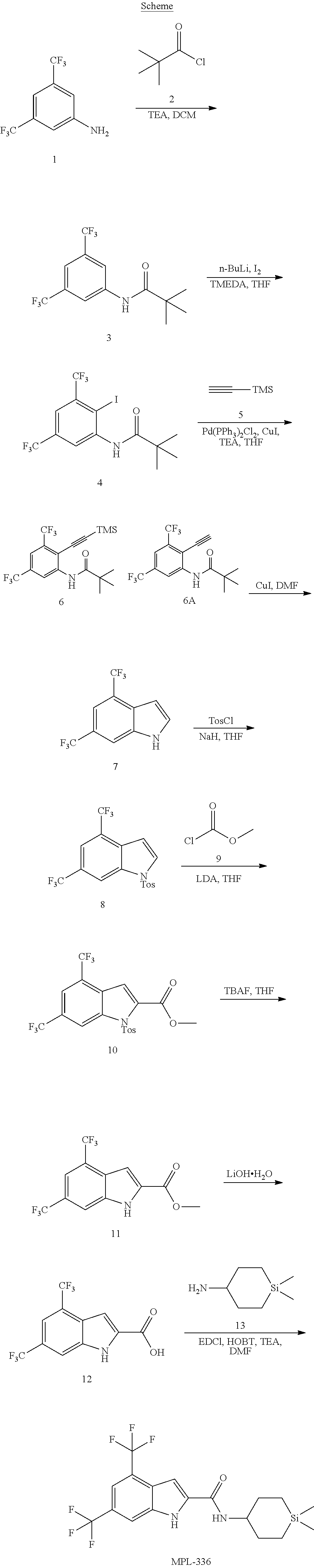

### Step 1. Synthesis of N-[3,5-bis(trifluoromethyl)phenyl]-2,2-dimethyl-propanamide

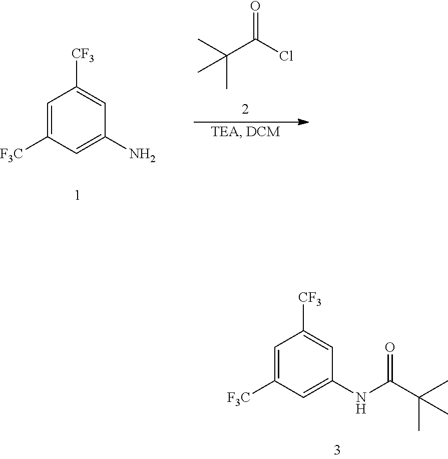

To a solution of 3,5-bis(trifluoromethyl)aniline (10.49 g, 45.78 mmol, 7.09 mL, 1 eq) and TEA (9.27 g, 91.57 mmol, 12.74 mL, 2 eq) in DCM (100 mL) was added 2,2-dimethylpropanoyl chloride (8.28 g, 68.68 mmol, 8.45 mL, 1.5 eq) dropwise at 0° C. The mixture was stirred at 20° C. for 30 min. TLC Showed the starting material was consumed. The reaction was quenched with saturated NaHCO₃ (200 mL) and extracted with DCM (150 mL×2). The organic layer was dried over Na₂SO₄ and filtered and concentrated under reduced pressure. The resulting residue was purified by column chromatography (SiO₂, 0-10% Ethyl acetate in petroleum ether). Compound N-[3,5-bis(trifluoromethyl)phenyl]-2,2-dimethyl-propanamide (14 g, 42.46 mmol, 92.74% yield, 95% purity) was obtained as a yellow solid. ¹H NMR was recorded.

### Step 2. Synthesis of N-[2-iodo-3,5-bis(trifluoromethyl)phenyl]-2,2-dimethyl-propanamide

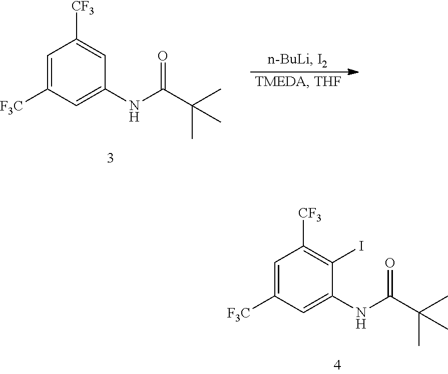

To a solution of N-[3,5-bis(trifluoromethyl)phenyl]-2,2-dimethyl-propanamide (8 g, 25.54 mmol, 1 eq) and TMEDA (5.94 g, 51.08 mmol, 7.71 mL, 2 eq) in THE (80 mL) was added n-BuLi (2.5 M in n-hexane, 25.54 mL, 2.5 eq) under N₂ at −78° C. The mixture was stirred at −78° C. for 30 min. Then a solution of I₂ (7.78 g, 30.65 mmol, 6.17 mL, 1.2 eq) in THE (20 mL) was added at −78° C. with stirring for 30 min. TLC showed the starting material remained and one new spot was formed. The mixture was quenched with saturated NH₄Cl (200 mL). The aqueous phase was extracted with EtOAc (100 mL×2). The combined organic layer was dried over Na₂SO₄ and filtered and concentrated under reduced pressure. The resulting residue was purified by column chromatography (SiO₂, 0-10% Ethyl acetate in petroleum ether). Compound N-[2-iodo-3,5-bis(trifluoromethyl)phenyl]-2,2-dimethyl-propanamide (4.4 g, 9.52 mmol, 31.06% yield, 95% purity) was obtained as a yellow solid.

### Step 3. Synthesis of N-[3,5-bis(trifluoromethyl)-2-(2-trimethylsilylethynyl)phenyl]-2,2-dimethyl-propanamide

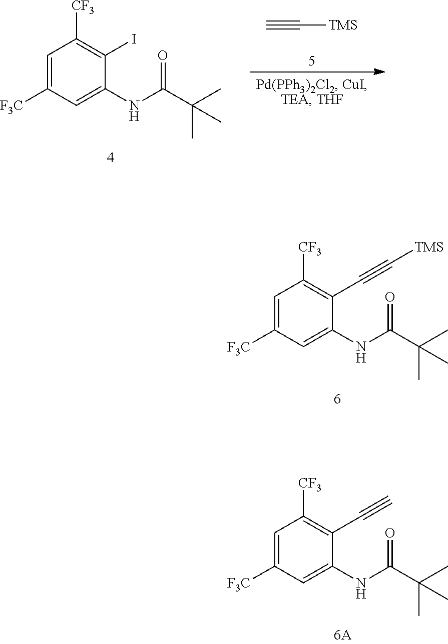

To a solution of N-[2-iodo-3,5-bis(trifluoromethyl)phenyl]-2,2-dimethyl-propanamide (4.4 g, 10.02 mmol, 1 eq) and ethynyl(trimethyl)silane (9.84 g, 100.20 mmol, 13.88 mL, 10 eq) in THE (60 mL) and TEA (14.54 g, 143.69 mmol, 20 mL, 14.34 eq) was added Pd(PPh₃)₂Cl₂ (703.28 mg, 1.00 mmol, 0.1 eq) and CuI (190.83 mg, 1.00 mmol, 0.1 eq) under N₂. The mixture was stirred at 80° C. for 12 hr. TLC showed the desired product. The mixture was filtered and the filtrated was concentrated under reduced pressure. The resulting residue was purified by column chromatography (SiO₂, 0-10% Ethyl acetate in petroleum ether). Compound N-[3,5-bis(trifluoromethyl)-2-(2-trimethylsilylethynyl)phenyl]-2,2-dimethyl-propanamide (2.8 g, 6.50 mmol, 64.84% yield, 95% purity) was obtained as a brown liquid. Compound N-[2-ethynyl-3,5-bis(trifluoromethyl)phenyl]-2,2-dimethyl-propanamide (500 mg, 1.41 mmol, 14.06% yield, 95% purity) was obtained as a brown liquid. ¹H NMR was recorded.

### Step 4. Synthesis of 4,6-bis(trifluoromethyl)-1H-indole

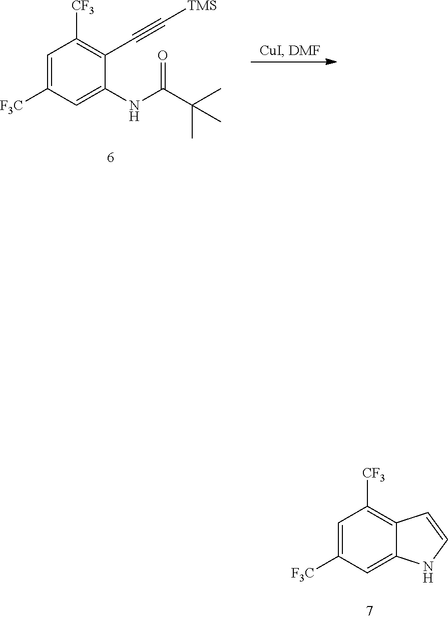

To a solution of N-[3,5-bis(trifluoromethyl)-2-(2-trimethylsilylethynyl)phenyl]-2,2-dimethyl-propanamide (2.8 g, 6.84 mmol, 1 eq) in THE (28 mL) was added TBAF (1 M in THF, 20.52 mL, 3 eq) under N₂. The mixture was stirred at 80° C. for 12 hr. TLC showed the starting material was consumed. The mixture was concentrated under reduced pressure. The residue was purified by column chromatography (SiO₂, 0-20% Ethyl acetate in petroleum ether). Compound 4,6-bis(trifluoromethyl)-1H-indole (1.7 g, 6.38 mmol, 93.29% yield, 95% purity) was obtained as a yellow liquid. ¹H NMR was recorded.

### Step 5. Synthesis of 1-(p-tolylsulfonyl)-4,6-bis(trifluoromethyl)indole

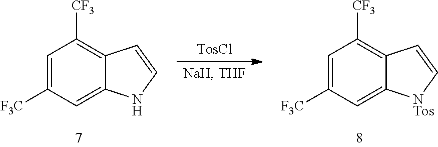

To a solution of 4,6-bis(trifluoromethyl)-1H-indole (1 g, 3.95 mmol, 1 eq) in THE (10 mL) was added NaH (237.00 mg, 5.93 mmol, 60% purity, 1.5 eq) with stirring for 30 min at 0° C., followed by TosCl (903.74 mg, 4.74 mmol, 1.2 eq). The mixture was stirred at 0° C. for 30 min. TLC showed the starting material was consumed. The mixture was poured into saturated NH₄Cl (20 mL) and extracted with EtOAc (20 mL×2). The combined organic layer was dried over Na₂SO₄, and then filtered and concentrated under reduced pressure. The residue was purified by column chromatography (SiO₂, 0-100% Ethyl acetate in petroleum ether). Compound 1-(p-tolylsulfonyl)-4,6-bis(trifluoromethyl)indole (600 mg, 1.40 mmol, 35.42% yield, 95% purity) was obtained as a yellow solid. ¹H NMR was recorded.

### Step 6. Synthesis of methyl 1-(p-tolylsulfonyl)-4,6-bis(trifluoromethyl)indole-2-carboxylate

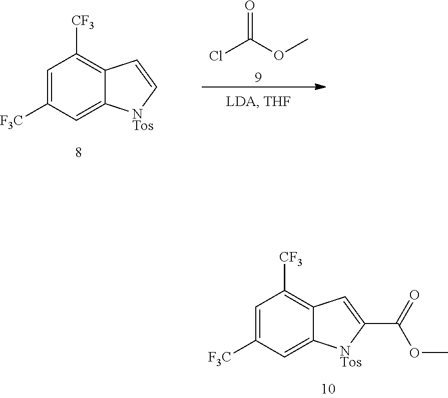

To a solution of 1-(p-tolylsulfonyl)-4,6-bis(trifluoromethyl)indole (600 mg, 1.47 mmol, 1 eq) in THE (10 mL) was added LDA (2 M, 1.10 mL, 1.5 eq) dropwise under N₂ at −60° C. The mixture was stirred at −60° C. for 30 min. Then methyl carbonochloridate (694.55 mg, 7.35 mmol, 569.31 uL, 5 eq) (0.860 g) was added dropwise to the solution with stirring for 30 min at −60° C. TLC showed the starting mateiral was consumed. The mixture was poured into saturated NH₄Cl (30 mL) and extracted with EtOAc (30 mL×2). The combined organic layer was dried over Na₂SO₄ and filtered and concentracted under reduced pressure. The resulting residue was purified by column chromatography (SiO₂, 0-10% Ethyl acetate in petroleum ether). Compound methyl 1-(p-tolylsulfonyl)-4,6-bis(trifluoromethyl)indole-2-carboxylate (300 mg, 644.65 umol, 43.85% yield) was obtained as a white solid. ¹H NMR was recorded.

### Step 7. Synthesis of methyl 4,6-bis(trifluoromethyl)-1H-indole-2-carboxylate

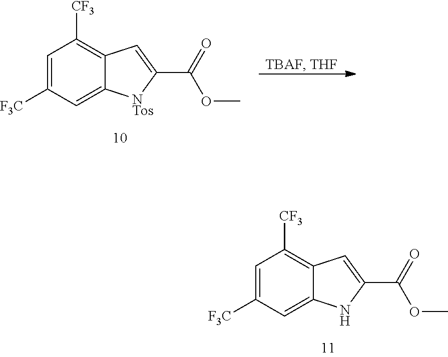

A solution of methyl 1-(p-tolylsulfonyl)-4,6-bis(trifluoromethyl)indole-2-carboxylate (300 mg, 644.65 umol, 1 eq) and TBAF (1 M in THF, 773.59 uL, 1.2 eq) in THE (5 mL) was stirred at 30° C. for 2 hr. TLC showed the starting material was consumed. The mixture was concentrated under reduced pressure to remove THF. The residue was poured into water (10 mL) with sonication for 30 min. The suspension was filtered. The filter cake was washed with water (10 mL) and collected. The residue was purified by column chromatography (SiO₂, 0-20% Ethyl acetate in petroleum ether). Compound methyl 4,6-bis(trifluoromethyl)-1H-indole-2-carboxylate (100 mg, 321.36 umol, 49.85% yield, 100% purity) was obtained as a white solid.

¹H NMR (500 MHz, CDCl₃) δ=9.47 (br, s, 1H), 7.95 (s, 1H), 7.71 (s, 1H), 7.45 (s, 1H), 4.01 (s, 3H).

### Step 8. Synthesis of methyl 4,6-bis(trifluoromethyl)-1H-indole-2-carboxylic acid

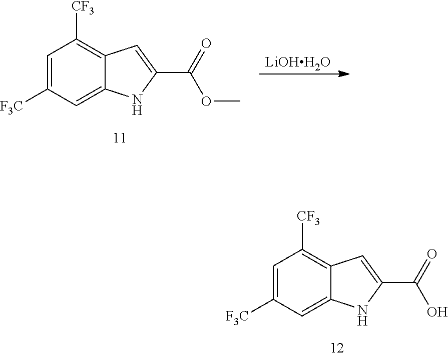 [SMILES: COC(=O)c1cc2c(C(F)(F)F)cc(C(F)(F)F)cc2[nH]1 ; "LiOH·H2O" ; O=C(O)c1cc2c(C(F)(F)F)cc(C(F)(F)F)cc2[nH]1]

To a solution of methyl 4,6-bis(trifluoromethyl)-1H-indole-2-carboxylate (100 mg, 321.36 umol, 1 eq) in THE (2.5 mL) was added a solution of LiOH.H₂O (134.84 mg, 3.21 mmol, 10 eq) in H₂O (2.5 mL). The mixture was stirred at 30° C. for 12 hr. TLC showed the starting material was consumed. The mixture was concentrated under reduced pressure to remove THF. The aqueous solution was adjusted to pH to 2 with aqueous HCl (6 M), and then filtered to collect solid. Compound 4,6-bis(trifluoromethyl)-1H-indole-2-carboxylic acid (90 mg, crude) was obtained as a white solid. The crude product was used for the next step without purification.

### Step 9. Synthesis of N-(1,1-dimethylsilinan-4-yl)-4,6-bis(trifluoromethyl)-1H-indole-2-carboxamide

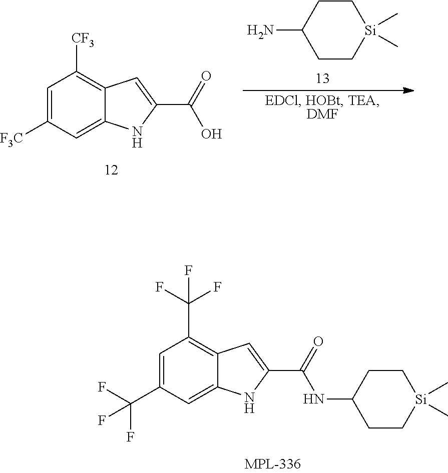

To a solution of 4,6-bis(trifluoromethyl)-1H-indole-2-carboxylic acid (30 mg, 100.96 umol, 1 eq) and 1,1-dimethylsilinan-4-amine (21.78 mg, 121.15 umol, 1.2 eq, HCl salt) in DMF (0.5 mL) was added a solution of EDCI (58.06 mg, 302.87 umol, 3 eq) and HOBt (40.93 mg, 302.87 umol, 3 eq) in DMF (0.5 mL), followed by TEA (51.08 mg, 504.79 umol, 70.26 uL, 5 eq). The mixture was stirred at 30° C. for 1 hr. LCMS showed the desired product was detected. The mixture was purified by prep-HPLC (column: YMC-Actus Triart C18 150*30 mm*5 um; mobile phase: A: 0.225% formic acid in water, B: CH₃CN, gradient: 70%-100% B over 11 min). Compound N-(1,1-dimethylsilinan-4-yl)-4,6-bis(trifluoromethyl)-1H-indole-2-carboxamide (24.1 mg, 57.05 umol, 56.51% yield, 100% purity) was obtained as a white solid.

LCMS (ESI), m/z 423.1[M+H]⁺

¹H NMR (500 MHz, DMSO-d6) δ=12.51 (s, 1H) 8.57 (d, J=8.24 Hz, 1H) 7.93 (s, 1H) 7.57 (s, 1H) 7.41 (s, 1H) 3.57-3.72 (m, 1H) 1.86-2.01 (m, 2H) 1.44-1.62 (m, 2H) 0.68 (br d, J=14.50 Hz, 2H) 0.53 (td, J=14.27, 4.73 Hz, 2H) −0.12-0.05 (m, 6H).

### Example 20, MPL-337

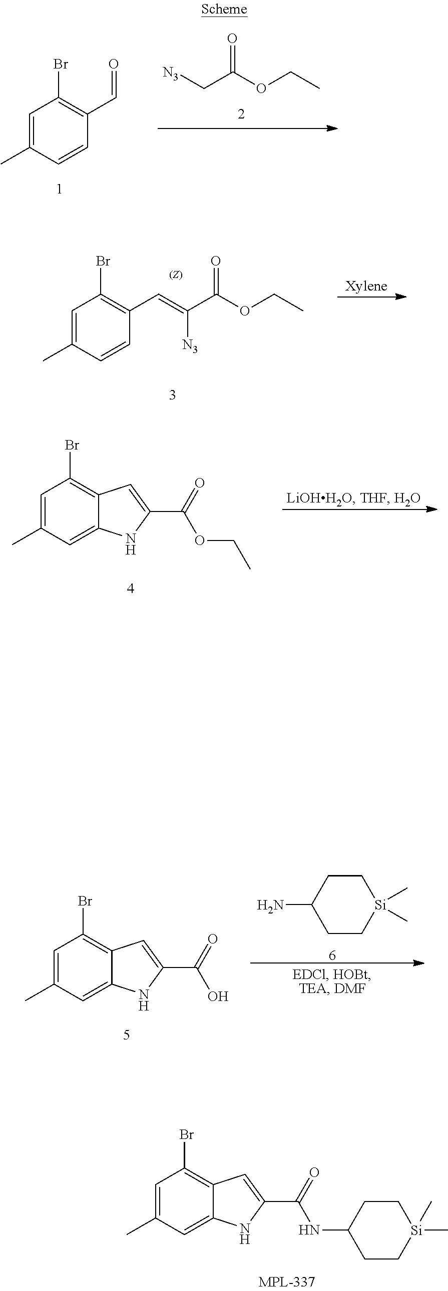

### Step 1. Synthesis of ethyl (Z)-2-azido-3-(2-bromo-4-methyl-phenyl)prop-2-enoate

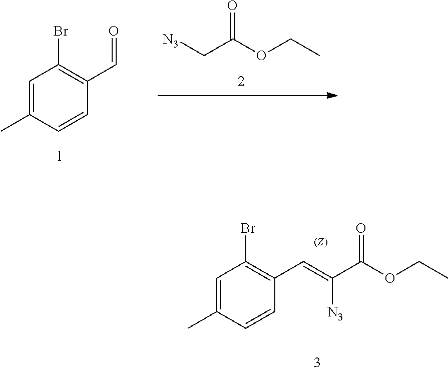

NaH (602.82 mg, 15.07 mmol, 60% purity, 3 eq) was added to EtOH (10 mL) in batches under N₂. The mixture was stirred at 20° C. until a clear solution formed and then cooled to −10° C. Then a solution of 2-bromo-4-methyl-benzaldehyde (1 g, 5.02 mmol, 1 eq) and ethyl 2-azidoacetate (1.95 g, 15.07 mmol, 2.12 mL, 3 eq) in THF (10 mL) was added dropwise. The reaction mixture was stirred at −10° C.˜0° C. for 2 hr. TLC (Petroleum ether: Ethyl acetate=5:1) indicated the reactant was consumed, and one major new spot formed. The reaction was quenched with saturated NH₄Cl (30 mL), and then extracted with EtOAc (20 mL×2). The combined organic layer was washed with brine (20 mL×2), dried over Na₂SO₄, and filtered and concentrated under reduced pressure. The resulting residue was purified by column chromatography (SiO2, 0-10% Ethyl acetate in petroleum ether). Compound ethyl (Z)-2-azido-3-(2-bromo-4-methyl-phenyl)prop-2-enoate (404 mg, 1.30 mmol, 25.93% yield) was obtained as a yellow oil.

### Step 2. Synthesis of ethyl 4-bromo-6-methyl-1H-indole-2-carboxylate

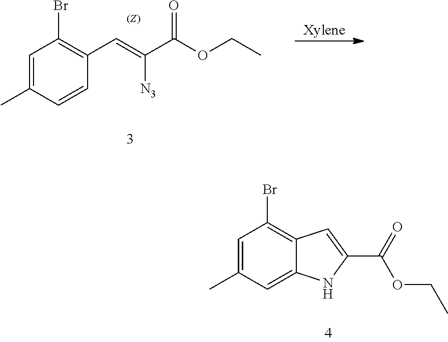 [SMILES: CCOC(=O)/C(=C/c1ccc(C)cc1Br)N=[N+]=[N-] ; "Xylene" ; CCOC(=O)c1cc2c(Br)cc(C)cc2[nH]1]

A mixture of ethyl (Z)-2-azido-3-(2-bromo-4-methyl-phenyl)prop-2-enoate (404 mg, 1.30 mmol, 1 eq) in xylene (2 mL) was stirred at 140° C. for 30 min. LCMS indicated desired mass was detected. The reaction mixture was concentrated under reduced pressure. The residue was purified by column chromatography (SiO₂, 0-7% Ethyl acetate in petroleum ether). Compound ethyl 4-bromo-6-methyl-1H-indole-2-carboxylate (117 mg, 393.96 umol, 30.24% yield, 95% purity) was obtained as a yellow solid.

LCMS (ESI) m/z: 283.9 [M+H]⁺

¹H NMR was recorded.

### Step 3. Synthesis of 4-bromo-6-methyl-1H-indole-2-carboxylic acid

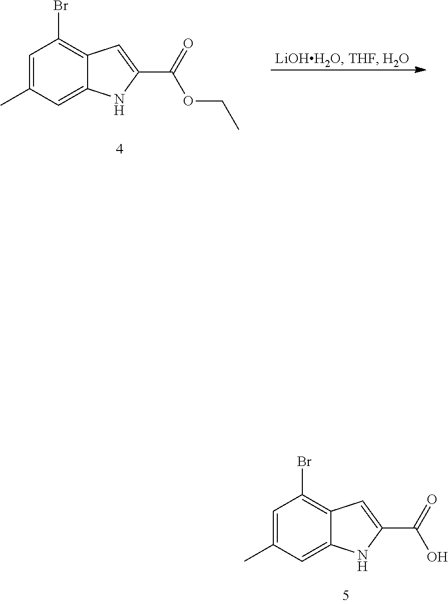

To a solution of ethyl 4-bromo-6-methyl-1H-indole-2-carboxylate (117 mg, 414.70 umol, 1 eq) in THF (2 mL) was added a solution of LiOH.H₂O (104.41 mg, 2.49 mmol, 6 eq) in H₂O (2 mL). The mixture was stirred at 80° C. for 12 hr. LCMS indicated desired mass was detected. The reaction mixture was concentrated under reduced pressure to remove THF. The aqueous residue was adjusted pH to 3-4 with aqueous HCl (1 N) and then filtered. The filter cake was washed with petroleum ether (15 mL) and dried under reduced pressure. Compound 4-bromo-6-methyl-1H-indole-2-carboxylic acid (73 mg, 258.58 umol, 62.35% yield, 90% purity) was obtained as a white solid. The crude product was used into the next step without further purification.

LCMS (ESI) m/z: 255.9 [M+H]⁺

¹H NMR (400 MHz, DMSO-d₆) δ=13.11 (br s, 1H), 12.01 (br s, 1H), 7.27-7.06 (m, 2H), 6.92 (d, J=1.2 Hz, 1H), 2.39 (s, 3H).

### Step 4. Synthesis of 4-bromo-N-(1,1-dimethylsilinan-4-yl)-6-methyl-1H-indole-2-carboxamide

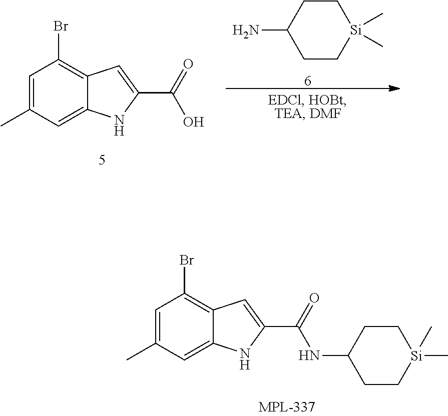

To a solution of 4-bromo-6-methyl-1H-indole-2-carboxylic acid (73 mg, 287.31 umol, 1 eq) and 1,1-dimethylsilinan-4-amine (61.98 mg, 344.77 umol, 1.2 eq, HCl salt) in DMF (2 mL) was added a solution of EDCI (165.23 mg, 861.93 umol, 3 eq) and HOBt (116.47 mg, 861.93 umol, 3 eq) in DMF (1 mL), followed by TEA (174.44 mg, 1.72 mmol, 239.94 uL, 6 eq). The mixture was stirred at 25° C. for 1 hr. LCMS indicated desired mass was detected. The mixture was purified by prep-HPLC (column: YMC-Actus Triart C18 150*30 mm*5 um; mobile phase: A: 0.225% formic acid in water, B: CH₃CN, gradient: 75%-100% B over 11 min). Compound 4-bromo-N-(1,1-dimethylsilinan-4-yl)-6-methyl-1H-indole-2-carboxamide (32.3 mg, 85.14 umol, 29.63% yield, 100% purity) was obtained as a white solid.

LCMS (ESI) m/z: 381.0 [M+H]⁺

¹H NMR (500 MHz, DMSO-d₆) δ=11.76 (br s, 1H), 8.35 (br d, J=8.1 Hz, 1H), 7.20 (s, 1H), 7.12 (br d, J=10.1 Hz, 2H), 3.71 (br d, J=8.7 Hz, 1H), 2.37 (s, 3H), 1.98 (br d, J=10.2 Hz, 2H), 1.66-1.51 (m, 2H), 0.77 (br d, J=14.3 Hz, 2H), 0.61 (dt, J=4.0, 14.0 Hz, 2H), 0.13-0.04 (m, 6H).

### Example 21, MPL-338

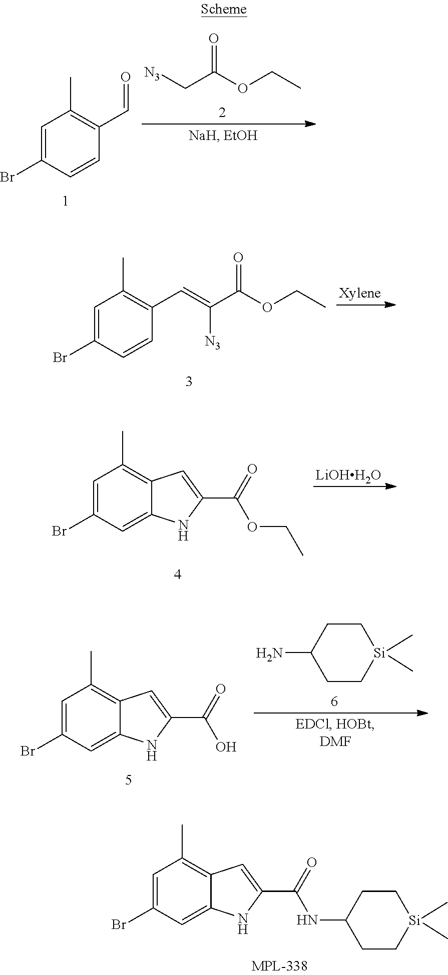

### Step 1. Synthesis of ethyl (Z)-2-azido-3-(4-bromo-2-methyl-phenyl)prop-2-enoate

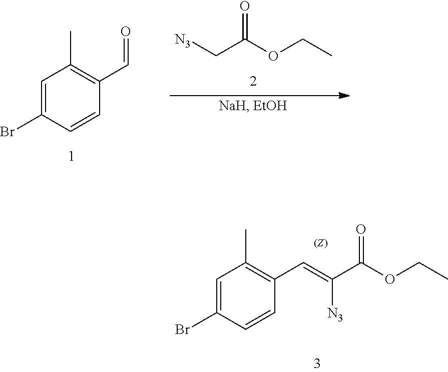

NaH (1.81 g, 45.22 mmol, 60% purity, 3 eq) was added to EtOH (20 mL) in batches. The mixture was stirred at 30° C. until a clear solution formed and then cooled to −10° C. A solution of 4-bromo-2-methyl-benzaldehyde (3 g, 15.07 mmol, 1 eq) and ethyl 2-azidoacetate (5.84 g, 45.22 mmol, 6.35 mL, 3 eq) in EtOH (20 mL) was added dropwise. The reaction mixture was stirred at −10° C.˜0° C. for 2 hr. TLC showed one new spot was detected. The reaction was quenched with saturated NH₄Cl (30 mL), and then extracted with EtOAc (50 mL×2). The combined organic layer was washed with brine (50 mL×2), dried over Na₂SO₄, filtered and concentrated under reduced pressure. Compound ethyl (Z)-2-azido-3-(4-bromo-2-methyl-phenyl)prop-2-enoate (4 g, crude) was obtained as a yellow solid. The crude product was used for the next step without further purification.

### Step 2. Synthesis of ethyl 6-bromo-4-methyl-1H-indole-2-carboxylate

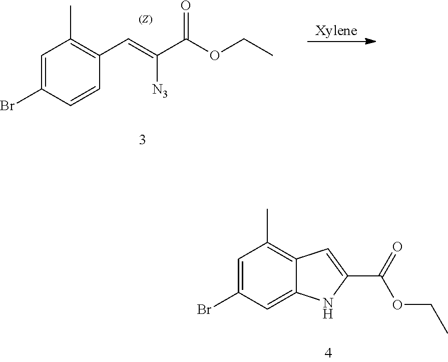 [SMILES: CCOC(=O)/C(=C/c1ccc(Br)cc1C)N=[N+]=[N-] ; "Xylene" ; CCOC(=O)c1cc2c(C)cc(Br)cc2[nH]1]

Ethyl (Z)-2-azido-3-(4-bromo-2-methyl-phenyl)prop-2-enoate (4 g, 12.90 mmol, 1 eq) in xylene (10 mL) was stirred at 150° C. for 20 min. The mixture was then cooled to room temperature, filtered to collect the cake. Compound ethyl 6-bromo-4-methyl-1H-indole-2-carboxylate (990 mg, 3.33 mmol, 25.85% yield, 95% purity) was obtained as a yellow solid. ¹H NMR was recorded.

### Step 3. Synthesis of 6-bromo-4-methyl-1H-indole-2-carboxylic acid

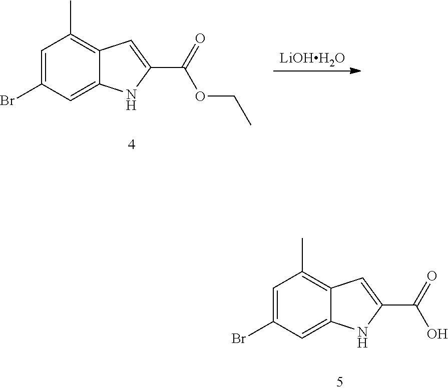 [SMILES: CCOC(=O)c1cc2c(C)cc(Br)cc2[nH]1 ; "LiOH·H2O" ; Cc1cc(Br)cc2[nH]c(C(=O)O)cc12]

To a solution of ethyl 6-bromo-4-methyl-1H-indole-2-carboxylate (100 mg, 354.44 umol, 1 eq) in THF (2 mL), a solution of LiOH.H₂O (59.49 mg, 1.42 mmol, 4 eq) in H₂O (2 mL) was added. The mixture was stirred at 80° C. for 12 hr. TLC indicated reactant was consumed completely and one new spot formed. The reaction mixture was concentrated under reduced pressure to remove THF. The aqueous residue was adjusted to pH to 2 with aqueous HCl (6 M) and then filtered. The filter cake was dried under reduced pressure. Compound 6-bromo-4-methyl-1H-indole-2-carboxylic acid (88 mg, 329.03 umol, 92.83% yield, 95% purity) was obtained as a white solid. ¹H NMR was recorded. The crude product was used for the next step without further purification.

### Step 4. Synthesis of 6-bromo-N-(1,1-dimethylsilinan-4-yl)-4-methyl-1H-indole-2-carboxamide

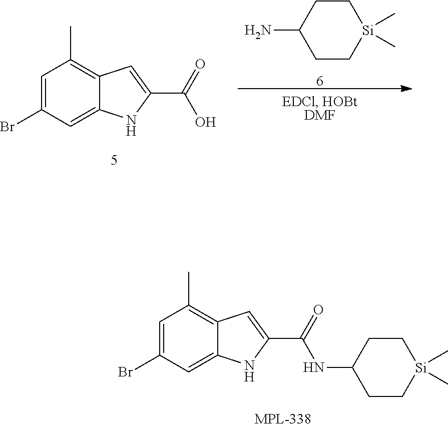

To a solution of 6-bromo-4-methyl-1H-indole-2-carboxylic acid (88 mg, 346.35 umol, 1 eq) and 1,1-dimethylsilinan-4-amine (74.71 mg, 415.62 umol, 1.2 eq, HCl salt) in DMF (1 mL) at 25° C. was added a solution of HOBt (140.40 mg, 1.04 mmol, 3 eq) and EDCI (199.19 mg, 1.04 mmol, 3 eq) in DMF (2 mL), followed by TEA (175.23 mg, 1.73 mmol, 241.04 uL, 5 eq). The reaction mixture was stirred at 25° C. for 12 hr. LCMS showed desired compound was detected. The mixture was purified by prep-HPLC (column: YMC-Actus Triart C18 150*30 mm*5 um; mobile phase: A: 0.225% formic acid in water, B: CH₃CN, gradient: 70%-95% B over 11 min). Compound 6-bromo-N-(1,1-dimethylsilinan-4-yl)-4-methyl-1H-indole-2-carboxamide (72.7 mg, 191.64 umol, 55.33% yield, 100% purity) was obtained as a white solid.

LCMS (ESI) m/z 379.0 [M+H]⁺

¹H NMR (500 MHz, CHLOROFORM-d) δ=9.55 (br s, 1H), 7.47 (s, 1H), 7.07 (s, 1H), 6.78 (d, J=1.5 Hz, 1H), 6.05 (br d, J=7.9 Hz, 1H), 4.04-3.84 (m, 1H), 2.62-2.46 (m, 3H), 2.22 (td, J=3.8, 9.4 Hz, 2H), 1.67-1.57 (m, 2H), 0.89-0.68 (m, 4H), 0.09 (d, J=17.4 Hz, 6H).

### Example 22, MPL-339

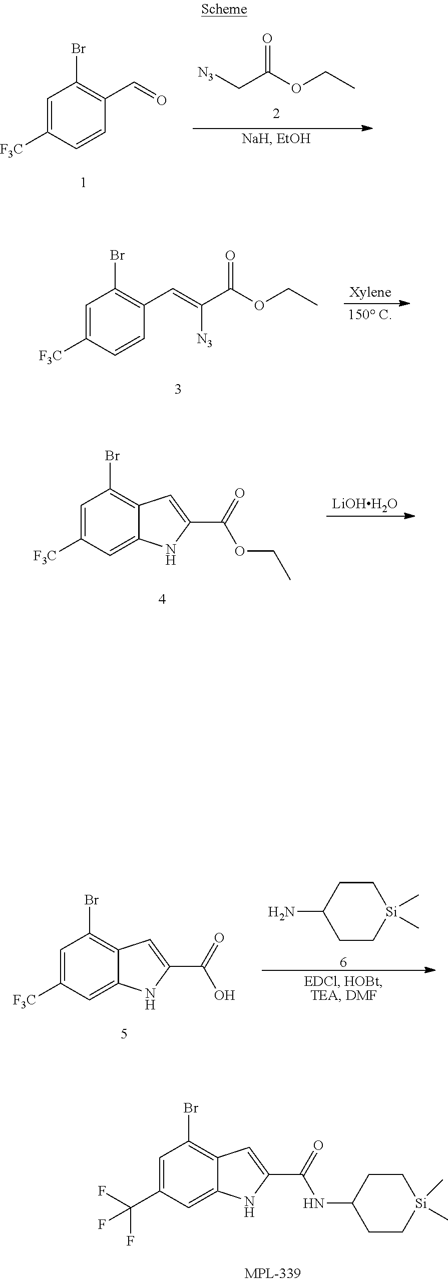

### Step 1. Synthesis of ethyl (Z)-2-azido-3-[2-bromo-4-(trifluoromethyl)phenyl]prop-2-enoate

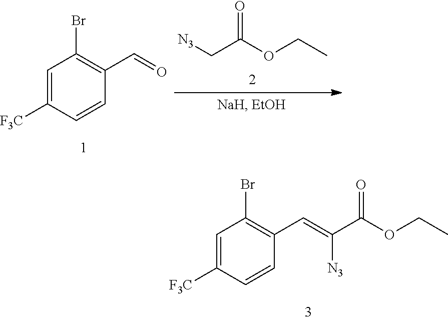

NaH (1.58 g, 39.52 mmol, 60% purity, 5 eq) was added to EtOH (30 mL) in batches. The mixture was stirred until a clear solution formed, and then cooled to −10° C. A mixture of 2-bromo-4-(trifluoromethyl)benzaldehyde (2 g, 7.90 mmol, 1 eq) and ethyl 2-azidoacetate (5.10 g, 39.52 mmol, 5.55 mL, 5 eq) was added dropwise at temperature below 0° C. The mixture was stirred at 0° C. for 2 hr. LCMS showed desired mass. TLC (Petroleum ether:EtOAc=20:1) indicated starting material was consumed completely. The mixture was poured into an ice-cooled saturated NH₄Cl (100 mL), and extracted with EtOAc (100 mL×2). The combined organic layer was dried with Na₂SO₄, filtered and concentrated to give a residue which was purified by flash silica gel chromatography (0-5% Ethyl acetate in petroleum ether). Compound ethyl (Z)-2-azido-3-[2-bromo-4-(trifluoromethyl)phenyl]prop-2-enoate (1.2 g, 1.98 mmol, 25.02% yield, 60% purity) was obtained as a light yellow oil. ¹H NMR was recorded.

### Step 2. Synthesis of ethyl 4-bromo-6-(trifluoromethyl)-1H-indole-2-carboxylate

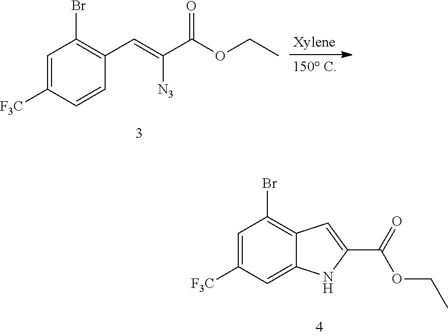 [SMILES: CCOC(=O)/C(=C/c1ccc(C(F)(F)F)cc1Br)N=[N+]=[N-] ; CCOC(=O)c1cc2c(Br)cc(C(F)(F)F)cc2[nH]1]

A solution of ethyl (Z)-2-azido-3-[2-bromo-4-(trifluoromethyl)phenyl]prop-2-enoate (1.2 g, 3.30 mmol, 1 eq) in xylene (10 mL) was stirred and refluxed at 150° C. for 0.5 hr. TLC (Petroleum ether:EtOAc=10:1) showed the reaction was messy. The reaction solution was cooled to 10° C. gradually. The reaction mixture was purified by flash silica gel chromatography (0-15% Ethyl acetate in petroleum ether). Compound ethyl 4-bromo-6-(trifluoromethyl)-1H-indole-2-carboxylate (110 mg, 261.82 umol, 7.94% yield, 80% purity) was obtained as a light yellow solid. ¹H NMR was recorded.

### Step 3. Synthesis of 4-bromo-6-(trifluoromethyl)-1H-indole-2-carboxylic acid

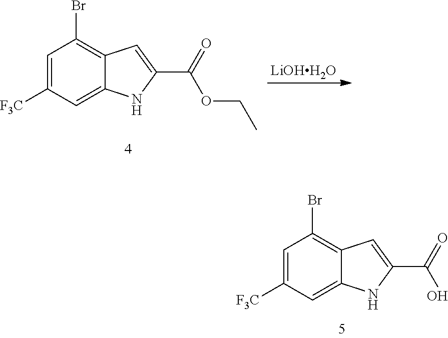

To a solution of ethyl 4-bromo-6-(trifluoromethyl)-1H-indole-2-carboxylate (110 mg, 327.28 umol, 1 eq) in EtOH (1 mL) was added a solution of LiOH (47.03 mg, 1.96 mmol, 6 eq) in H₂O (1 mL). The mixture was stirred at 60° C. for 12 hr. LCMS showed reactant 4 was consumed completely. TLC (Petroleum ether:EtOAc=5:1) showed one spot on baseline. The reaction mixture was concentrated under reduced pressure. The resulting residue was diluted with water (5 mL) and adjusted to pH to 3 with HCl (6M in water). The solid was collected by filtration and washed with—water (5 mL). Compound 4-bromo-6-(trifluoromethyl)-1H-indole-2-carboxylic acid (65 mg, 200.45 umol, 61.25% yield, 95% purity) was obtained as a light yellow soild.

¹H NMR (500 MHz, DMSO-d₆) δ=12.54 (br s, 1H), 7.76 (s, 1H), 7.58 (s, 1H), 7.00 (s, 1H).

### Step 4. Synthesis of 4-bromo-N-(1,1-dimethylsilinan-4-yl)-6-(trifluoromethyl)-1H-indole-2-carboxamide

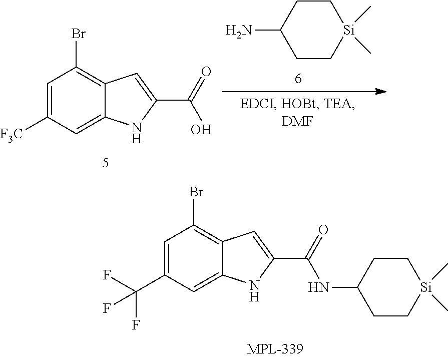

To a solution of 4-bromo-6-(trifluoromethyl)-1H-indole-2-carboxylic acid (60 mg, 194.77 umol, 1 eq) and 1,1-dimethylsilinan-4-amine (42.01 mg, 233.72 umol, 1.2 eq, HCl salt) in DMF (1 mL) was added a solution of EDCI (74.68 mg, 389.54 umol, 2 eq) and HOBt (52.64 mg, 389.54 umol, 2 eq) in DMF (1 mL), followed by TEA (78.83 mg, 779.08 umol, 108.44 uL, 4 eq). The mixture was stirred at 20° C. for 2 hr. LCMS showed reactant 5 was consumed completely and one main peak with desired mass. The mixture was diluted with MeOH (1.5 mL) until a clear solution formed and purified by prep-HPLC (column: YMC-Actus Triart C18 150*30 mm*5 um; mobile phase: A: 0.225% formic acid in water, B: CH₃CN, gradient: 75%-100% B over 11 min). Compound 4-bromo-N-(1,1-dimethylsilinan-4-yl)-6-(trifluoromethyl)-1H-indole-2-carboxamide (29 mg, 66.52 umol, 34.15% yield, 99.4% purity) was obtained as a white solid.

LCMS (ESI) m/z 433.1 [M+H]⁺

¹H NMR (500 MHz, DMSO-d₆) δ=12.32 (s, 1H), 8.50 (d, J=8.2 Hz, 1H), 7.66 (s, 1H), 7.46 (s, 1H), 7.25 (s, 1H), 3.69-3.58 (m, 1H), 1.95-1.84 (m, 2H), 1.60-1.45 (m, 2H), 0.69 (br d, J=14.5 Hz, 2H), 0.53 (dt, J=4.7, 14.2 Hz, 2H), 0.00 (s, 3H), −0.06 (s, 3H).

### Example 23, MPL-340

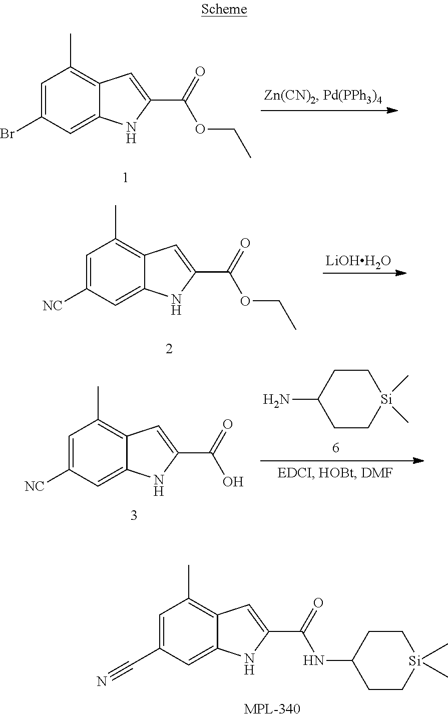

### Step 1. Synthesis of ethyl 6-cyano-4-methyl-1H-indole-2-carboxylate

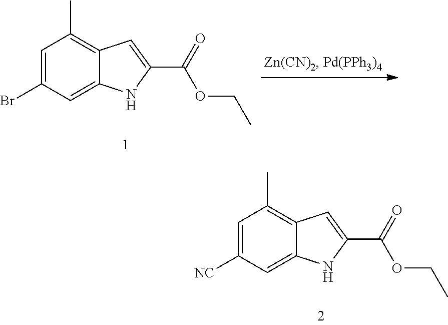 [SMILES: CCOC(=O)c1cc2c(C)cc(Br)cc2[nH]1 ; CCOC(=O)c1cc2c(C)cc(C#N)cc2[nH]1]

To a mixture of ethyl 6-bromo-4-methyl-1H-indole-2-carboxylate (100 mg, 354.44 umol, 1 eq) in DMF (5 mL) was added Zn(CN)₂ (62.43 mg, 531.66 umol, 33.75 uL, 1.5 eq). The mixture was purged with N₂ and Pd(PPh₃)₄ (81.92 mg, 70.89 umol, 0.2 eq) was then added under N₂. The mixture was stirred at 100° C. for 12 hr. LCMS showed desired compound was detected. The mixture was filtered. The cake was washed with EtOAc (10 mL×2). The combined filtrate was diluted with EtOAc (20 mL) and extracted with 3% LiCl in water (20 mL×3). The combined organic layer was washed with brine (20 mL×3), dried over Na₂SO₄, and then filtered and concentrated under reduced pressure. The resulting residue was purified by column chromatography (SiO₂, 0-20% Ethyl acetate in petroleum ether). Compound ethyl 6-cyano-4-methyl-1H-indole-2-carboxylate (40 mg, 162.98 umol, 45.98% yield, 93% purity) was obtained as a white solid. ¹H NMR was recorded.

### Step 2. Synthesis of 6-cyano-4-methyl-1H-indole-2-carboxylic acid

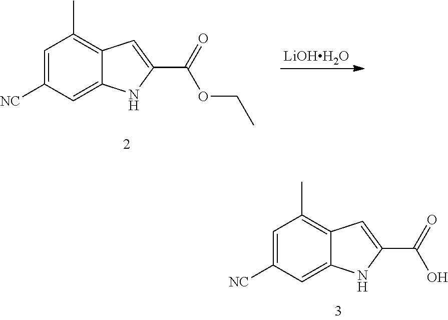 [SMILES: CCOC(=O)c1cc2c(C)cc(C#N)cc2[nH]1 ; "LiOH·H2O" ; Cc1cc(C#N)cc2[nH]c(C(=O)O)cc12]

To a solution of ethyl 6-cyano-4-methyl-1H-indole-2-carboxylate (40 mg, 175.25 umol, 1 eq) in THE (1 mL) was added a solution of LiOH.H₂O (44.12 mg, 1.05 mmol, 6 eq) in H₂O (1 mL). The mixture was stirred at 25° C. for 12 hr. TLC showed one major new spot with higher polarity. The reaction mixture was concentrated under reduced pressure to remove THF, and then adjusted to pH to 2 with HCl (6 M in water) and filtered. The filter cake was dried under reduced pressure. Compound 6-cyano-4-methyl-1H-indole-2-carboxylic acid (30 mg, 134.87 umol, 76.96% yield, 90% purity) was obtained as a white solid. The crude product was used for the next step without further purification.

¹H NMR (500 MHz, DMSO-d₆) δ=13.49-13.27 (m, 1H), 12.32 (s, 1H), 7.71 (s, 1H), 7.24 (s, 1H), 7.21 (s, 1H), 2.56-2.53 (m, 3H)

### Step 3. Synthesis of 6-cyano-N-(1,1-dimethylsilinan-4-yl)-4-methyl-1H-indole-2-carboxamide

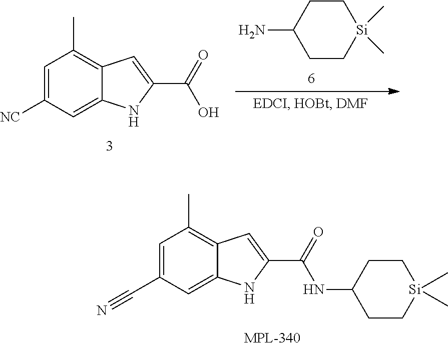

To a solution of 6-cyano-4-methyl-1H-indole-2-carboxylic acid (30 mg, 149.86 umol, 1 eq) and 1,1-dimethylsilinan-4-amine (29.63 mg, 164.84 umol, 1.1 eq, HCl salt) in DMF (1 mL) was added a solution of HOBt (60.75 mg, 449.57 umol, 3 eq) and EDCI (86.18 mg, 449.57 umol, 3 eq) in DMF (1 mL) with stirring, and followed by TEA (75.82 mg, 749.28 umol, 104.29 uL, 5 eq). The mixture was stirred at 25° C. for 2 hr. LCMS showed desired compound was detected. The mixture was purified by prep-HPLC (column: YMC-Actus Triart C18 150*30 mm*5 um; mobile phase: A: 0.225% formic acid in water, B: CH₃CN, gradient: 60%-90% B over 11 min). Compound 6-cyano-N-(1,1-dimethylsilinan-4-yl)-4-methyl-1H-indole-2-carboxamide (37.1 mg, 113.99 umol, 76.06% yield, 100% purity) was obtained as a white solid.

LCMS (ESI) m/z 326.0 [M+H]⁺

¹H NMR (500 MHz, DMSO-d₆) δ=12.05 (s, 1H), 8.44 (d, J=8.2 Hz, 1H), 7.68 (s, 1H), 7.32 (d, J=1.2 Hz, 1H), 7.17 (s, 1H), 3.80-3.65 (m, 1H), 2.52 (s, 3H), 2.05-1.93 (m, 2H), 1.67-1.52 (m, 2H), 0.78 (br d, J=14.5 Hz, 2H), 0.62 (dt, J=4.7, 14.1 Hz, 2H), 0.12-0.00 (m, 6H).

### Example 24, MPL-344

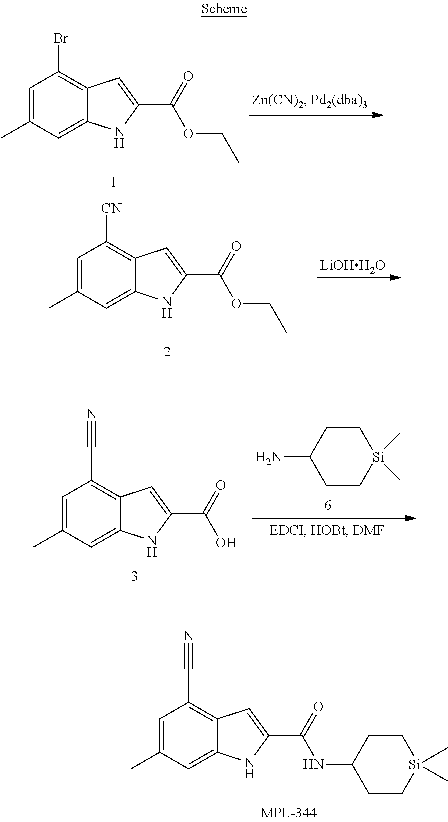

### Step 1. Synthesis of ethyl 4-cyano-6-methyl-1H-indole-2-carboxylate

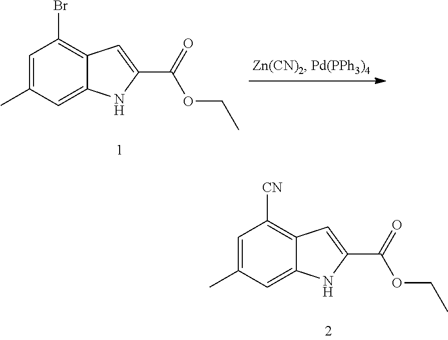 [SMILES: CCOC(=O)c1cc2c(Br)cc(C)cc2[nH]1 ; CCOC(=O)c1cc2c(C#N)cc(C)cc2[nH]1]

A mixture of ethyl 4-bromo-6-methyl-1H-indole-2-carboxylate (357 mg, 1.27 mmol, 1 eq) and Zn(CN)₂ (237.74 mg, 2.02 mmol, 128.51 uL, 1.6 eq) in DMF (3 mL) was degassed and purged with N₂ for 3 times, and then Pd(PPh₃)₄ (292.44 mg, 253.07 umol, 0.2 eq) was added. The mixture was stirred at 100° C. for 12 hr under N₂ atmosphere. TLC (Petroleum ether. Ethyl acetate=5:1) indicated reactant 1 was consumed completely and one new spot formed. The reaction mixture was filtered to obtain filtrate. The residue was diluted with Ethyl acetate (30 mL) and washed with H₂O (30 mL×2) and 3% LiCl in water (30 mL×2). The combined organic layer was washed with brine (30 mL×2), dried over Na₂SO₄, and then filtered and concentrated under reduced pressure. The resulting residue was purified by column chromatography (SiO₂, 0-100% Ethyl acetate in petroleum ether). Compound ethyl 4-cyano-6-methyl-1H-indole-2-carboxylate (84 mg, 349.62 umol, 27.63% yield, 95% purity) was obtained as a white solid.

### Step 2. Synthesis of 4-cyano-6-methyl-1H-indole-2-carboxylic acid

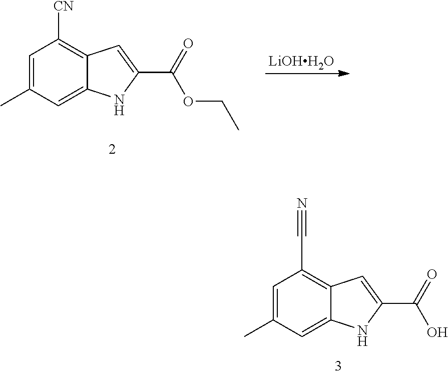 [SMILES: CCOC(=O)c1cc2c(C#N)cc(C)cc2[nH]1 ; "LiOH·H2O" ; Cc1cc(C#N)c2cc(C(=O)O)[nH]c2c1]

To a solution of ethyl 4-cyano-6-methyl-1H-indole-2-carboxylate (84 mg, 368.02 umol, 1 eq) in THE (2 mL) was added a solution of LiOH.H₂O (92.66 mg, 2.21 mmol, 6 eq) in H₂O (2 mL). The mixture was stirred at 30° C. for 12 hr. LCMS showed desired mass. The reaction mixture was concentrated under reduced pressure to remove THF, and then adjusted to pH to 3-4 with HCl (6 N in water) and filtered. The filter cake was washed with petroleum ether (15 mL) and dried under reduced pressure. Compound 4-cyano-6-methyl-1H-indole-2-carboxylic acid (57 mg, 256.25 umol, 69.63% yield, 90% purity) was obtained as a white solid. The crude product was used for the next step without further purification.

LCMS (ESI) m/z: 201.1 [M+H]⁺

¹H NMR (500 MHz, DMSO-d₆) δ=13.32 (br s, 1H), 12.30 (s, 1H), 7.56 (s, 1H), 7.50 (s, 1H), 7.06 (d, J=1.1 Hz, 1H), 2.44 (s, 3H).

### Step 3. Synthesis of 4-cyano-N-(1,1-dimethylsilinan-4-yl)-6-methyl-1H-indole-2-carboxamide

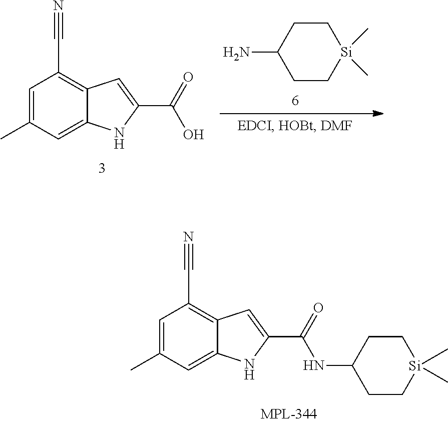

To a solution of 4-cyano-6-methyl-1H-indole-2-carboxylic acid (57 mg, 284.73 umol, 1 eq) and 1,1-dimethylsilinan-4-amine (48.96 mg, 341.67 umol, 1.2 eq, HCl salt) in DMF (2 mL) was added a solution of EDCI (163.75 mg, 854.18 umol, 3 eq) and HOBt (115.42 mg, 854.18 umol, 3 eq) in DMF (0.5 mL), followed by TEA (172.87 mg, 1.71 mmol, 237.78 uL, 6 eq). The reaction mixture was stirred at 20° C. for 1 hr. LCMS indicated that desired mass was detected. The reaction mixture was filtered to obtain filtrate, which was purified by prep-HPLC (column: Phenomenex Synergi C18 150*30 mm*4 um; mobile phase: A: 0.05% HCl in water, B: CH₃CN, gradient: 62%-82% B over 9 min). Compound 4-cyano-N-(1,1-dimethylsilinan-4-yl)-6-methyl-1H-indole-2-carboxamide (12.3 mg, 37.79 umol, 13.27% yield, 100% purity) was obtained as a white solid.

LCMS (ESI) m/z: 326.1 [M+H]⁺

¹H NMR (500 MHz, DMSO-d₆) δ=12.04 (s, 1H), 8.43 (d, J=8.2 Hz, 1H), 7.53 (s, 1H), 7.43 (s, 1H), 7.35 (d, J=1.4 Hz, 1H), 3.77-3.67 (m, 1H), 2.42 (s, 3H), 2.04-1.95 (m, 2H), 1.65-1.54 (m, 2H), 0.78 (br d, J=14.5 Hz, 2H), 0.61 (dt, J=4.7, 14.2 Hz, 2H), 0.09 (s, 3H), 0.03 (s, 3H).

### Example 25, MPL-360

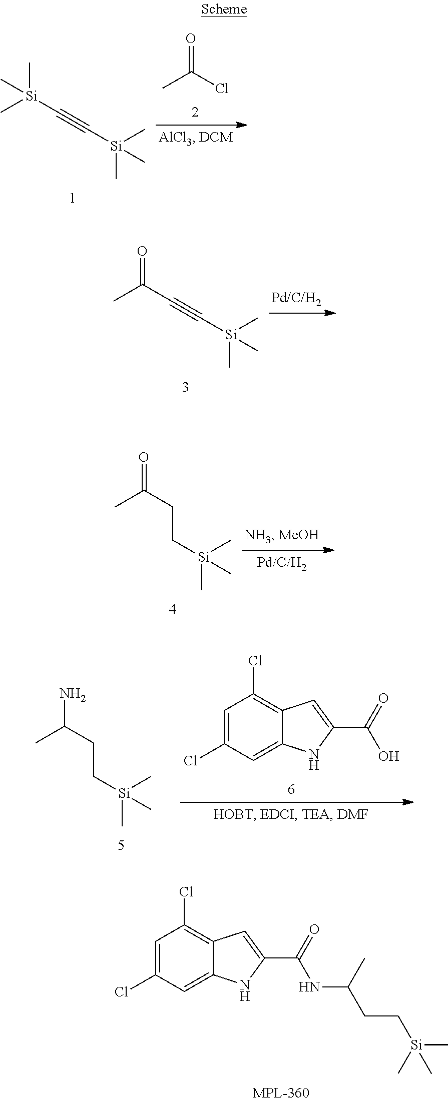

### Step 1. Synthesis of 4-trimethylsilylbut-3-yn-2-one

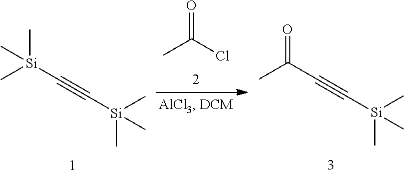

AlCl₃ (3.91 g, 29.34 mmol, 1.60 mL, 1 eq) was suspended in DCM (30 mL) and cooled in an ice bath. A solution of trimethyl(2-trimethylsilylethynyl)silane (5 g, 29.34 mmol, 6.65 mL, 1 eq) and acetyl chloride (2.30 g, 29.34 mmol, 2.09 mL, 1 eq) in DCM (60 mL) was added to the suspension dropwise from an addition funnel over 40 min. The dark brownish-red solution was stirred at 0° C. for 30 min. The ice bath was then removed. The mixture was stirred at 15° C. for 50 min. TLC showed the desired product was detected. The reaction was cooled to 0° C. and quenched by slow addition of 1 N HCl in water (75 mL). The acidic solution was extracted with DCM (2×150 mL). The combined organic layer was dried over Na₂SO₄ and filtered and concentrated under reduced pressure. Compound 4-trimethylsilylbut-3-yn-2-one (4 g, 27.09 mmol, 92.33% yield, 95% purity) was obtained as a brown liquid. ¹H NMR was recorded.

### Step 2. Synthesis of 4-trimethylsilylbutan-2-one

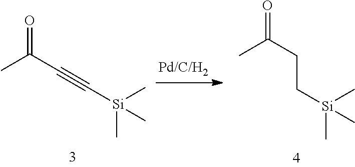

To a solution of 4-trimethylsilylbut-3-yn-2-one (1 g, 7.13 mmol, 1 eq) in pentane (10 mL) was added Pd/C (0.1 g, 93.97 umol, 10% purity, 1.32e-2 eq) under N₂. The suspension was degassed under vacuum and purged with H₂ several times. The mixture was stirred under H₂ (15 psi) at 10° C. for 12 hr. ¹H NMR showed the desired product was detected. The reaction mixture was filtered. The filter cake was washed with MeOH (10 mL). The filtrates were combined. Compound 4-trimethylsilylbutan-2-one (1.03 g, crude) was obtained in MeOH and pentane as a yellow liquid, which was used for the next step directly.

### Step 3. Synthesis of 4-trimethylsilylbutan-2-amine

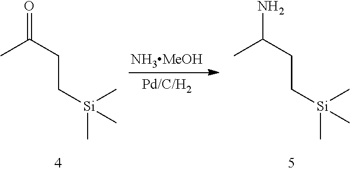

NH₃.MeOH (7 M, 10.20 mL, 5 eq) was added to a solution 4-trimethylsilylbutan-2-one (2.06 g, 14.28 mmol, 1 eq) in MeOH and pentane from previous step. The mixture was stirred for 2 hr at 15° C. Then NaBH(OAc)₃ (6.05 g, 28.55 mmol, 2 eq) was added. The mixture was stirred at 15° C. for 2 hr. TLC showed a new spot formed. The mixture was quenched with ice water (20 mL) and concentrated under reduced pressure to remove organic solvents. The aqueous solution was extracted with a mixed solvent of DCM and MeOH (10:1, 20 mL×2). The combined organic layer was dried over Na₂SO₄ and filtered. HCl in MeOH (4 M, 20 mL) was added to the filtrate and concentrated under reduced pressure to remove solvent. The resulting residue was triturated with EtOAc (20 mL) for 30 min with sonication. Compound 4-trimethylsilylbutan-2-amine (400 mg, 2.09 mmol, 14.64% yield, 95% purity, HCl) was obtained as a white solid.

¹H NMR (500 MHz, DMSO-d₆) δ=−0.07-0.09 (m, 9H), 0.41-0.56 (m, 2H), 1.16-1.18 (m, 3H), 1.35-1.50 (m, 1H), 1.53-1.68 (m, 1H), 2.51 (br s, 4H), 3.03 (sxt, J=6.47 Hz, 1H), 8.03 (br s, 3H)

### Step 4: Synthesis of 4,6-dichloro-N-(1-methyl-3-trimethylsilyl-propyl)-1H-indole-2-carboxamide

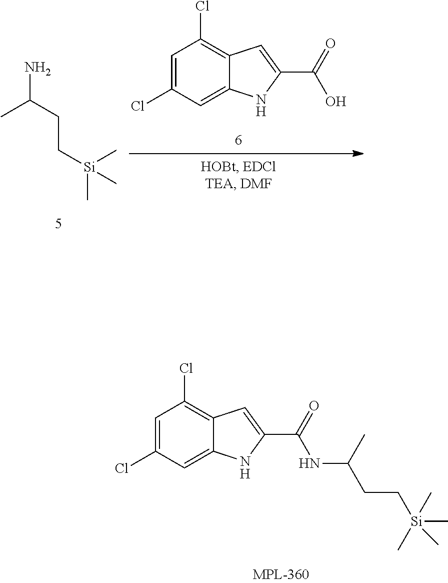

To a solution of 4,6-dichloro-1H-indole-2-carboxylic acid (50 mg, 217.35 umol, 1 eq) and 4-trimethylsilylbutan-2-amine (37.90 mg, 260.82 umol, 1.2 eq, HCl salt) in DMF (0.5 mL) was added a solution of EDCI (62.50 mg, 326.02 umol, 1.5 eq) and HOBt (44.05 mg, 326.02 umol, 1.5 eq) in DMF (0.5 mL), followed by TEA (131.96 mg, 1.30 mmol, 181.51 uL, 6 eq). The mixture was stirred at 20° C. for 1 hr. LCMS showed the desired product was detected. The mixture was filtered and the filtrate was purified by prep-HPLC (column: YMC-Actus Triart C18 150*30 mm*5 um; mobile phase: A: 0.225% formic acid in water, B: CH₃CN, gradient: 80%-100% B over 11 min). Compound 4,6-dichloro-N-(1-methyl-3-trimethylsilyl-propyl)-1H-indole-2-carboxamide (38.7 mg, 107.11 umol, 49.28% yield, 98.9% purity) was obtained as a yellow solid.

LCMS (ESI), m/z 357.1[M+H]⁺

¹H NMR (500 MHz, DMSO-d₆) δ ppm 0.00 (s, 9H) 0.37-0.63 (m, 2H) 1.17 (d, J=6.56 Hz, 3H) 1.36-1.65 (m, 2H) 3.96 (dt, J=14.08, 7.08 Hz, 1H) 7.24 (d, J=1.37 Hz, 1H) 7.32 (s, 1H) 7.42 (s, 1H) 8.39 (d, J=8.39 Hz, 1H) 12.07 (br s, 1H).

### Example 26, MPL-383

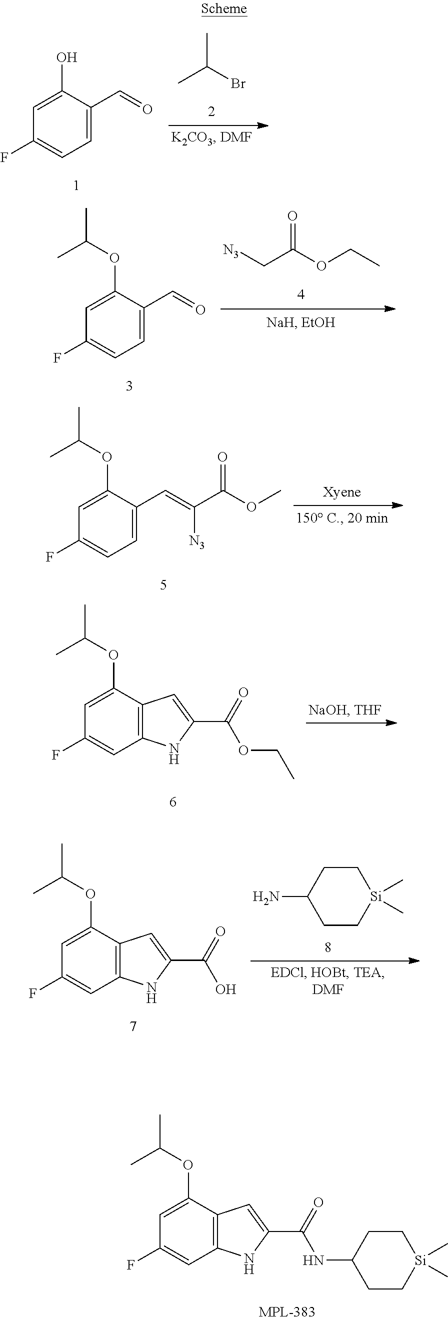

### Step 1. Synthesis of 4-fluoro-2-isopropoxy-benzaldehyde

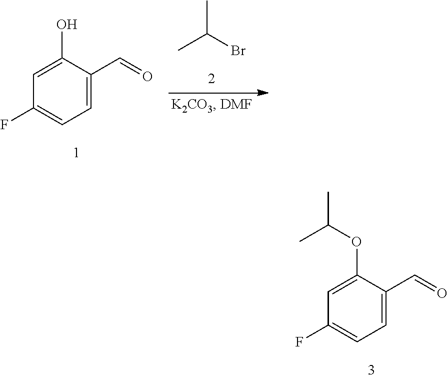

To a solution of 4-fluoro-2-hydroxy-benzaldehyde (2 g, 14.27 mmol, 1 eq) in DMF (20 mL) was added K₂CO₃ (3.95 g, 28.55 mmol, 2 eq) and 2-bromopropane (3.51 g, 28.55 mmol, 2.68 mL, 2 eq). The mixture was stirred at 100° C. for 12 hr. TLC (Petroleum ether. Ethyl acetate=5:1) indicated new spots formed. The mixture was poured into a mixture of water and EtOAc (100 mL). The aqueous layer and organic layer were separated. The aqueous layer was extracted with EtOAc (2×50 mL). The combined organic layer was dried over Na₂SO₄, and then filtered and concentrated. The resulting residue was purified by column chromatography (SiO₂, 0-3% Ethyl acetate in petroleum ether). Compound 4-fluoro-2-isopropoxy-benzaldehyde (2.23 g, 11.62 mmol, 81.42% yield, 95% purity) was obtained as a white solid. ¹H NMR was recorded.

### Step 2. Synthesis of ethyl (Z)-2-azido-3-(4-fluoro-2-isopropoxy-phenyl)prop-2-enoate

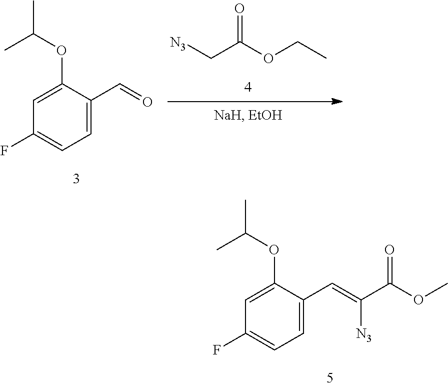

NaH (1.51 g, 37.87 mmol, 60% purity, 3 eq) was added to EtOH (10 mL) in batches under N₂. The mixture was stirred at 20° C. until a clear solution formed and then cooled to −10° C. A solution of 4-fluoro-2-isopropoxy-benzaldehyde (2.3 g, 12.62 mmol, 1 eq) and ethyl 2-azidoacetate (4.89 g, 37.87 mmol, 5.32 mL, 3 eq) in THE (20 mL) was then added dropwise. The reaction mixture was stirred at −10° C.˜ 0° C. for 2 hr. TLC (Petroleum ether: Ethyl acetate=3:1) indicated compound 3 was consumed completely and one new spot formed. The reaction was quenched with HCl (1 N in water, 60 mL), and then extracted with EtOAc (50 mL×2). The combined organic layer was washed with brine (30 mL×2), dried over Na₂SO₄, filtered and concentrated under reduced pressure. The resulting residue was purified by column chromatography (SiO₂, 0-10% Ethyl acetate in petroleum ether). Compound ethyl (Z)-2-azido-3-(4-fluoro-2-isopropoxy-phenyl) prop-2-enoate (2.4 g, 4.09 mmol, 32.41% yield, 50% purity) was obtained as a yellow solid, which was used for the next step without further purification.

### Step 3. Synthesis of ethyl 6-fluoro-4-isopropoxy-1H-indole-2-carboxylate

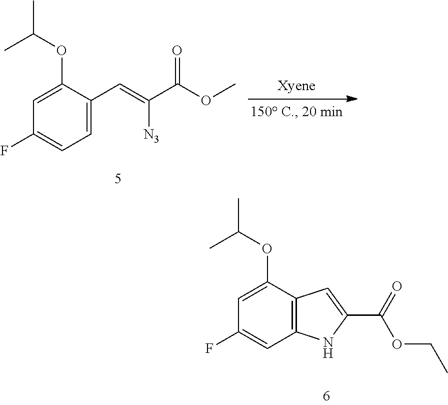 [SMILES: COC(=O)/C(=C/c1ccc(F)cc1OC(C)C)N=[N+]=[N-] ; CCOC(=O)c1cc2c(OC(C)C)cc(F)cc2[nH]1]

A solution of ethyl (Z)-2-azido-3-(4-fluoro-2-isopropoxy-phenyl) prop-2-enoate (945.66 mg, 3.22 mmol, 1 eq) in xylene (5 mL) was stirred at 150° C. for 30 min. LCMS showed desired mass. The reaction mixture was concentrated under reduced pressure to remove solvent. The residue was purified by column chromatography (SiO₂, 0-2% Ethyl acetate in petroleum ether). Compound ethyl 6-fluoro-4-isopropoxy-1H-indole-2-carboxylate (602 mg, 2.16 mmol, 66.86% yield, 95% purity) was obtained as a white solid.

LCMS (ESI) m/z: 266.1 [M+H]⁺

¹H NMR was recorded.

### Step 4. Synthesis of 6-fluoro-4-isopropoxy-1H-indole-2-carboxylic acid

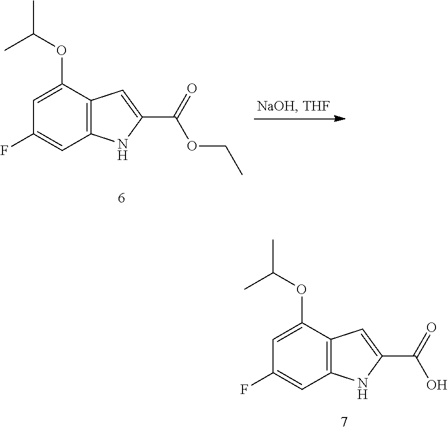 [SMILES: CCOC(=O)c1cc2c(OC(C)C)cc(F)cc2[nH]1 ; CC(C)Oc1cc(F)cc2[nH]c(C(=O)O)cc12]

To a solution of ethyl 6-fluoro-4-isopropoxy-1H-indole-2-carboxylate (600 mg, 2.26 mmol, 1 eq) in THF (3 mL) was added a solution of LiOH.H₂O (569.47 mg, 13.57 mmol, 6 eq) in H₂O (3 mL). The mixture was heated at 80° C. for 12 hr. LCMS showed desired mass. The reaction mixture was concentrated under reduced pressure to remove THF, and then adjusted to pH to 3-4 with HCl (6 N in water) and filtered. The filter cake was washed with petroleum ether (30 mL and dried under reduced pressure. Compound 6-fluoro-4-isopropoxy-1H-indole-2-carboxylic acid (480 mg, 1.92 mmol, 84.99% yield, 95% purity) was obtained as a white solid, which was used for the next step without further purification.

LCMS (ESI) m/z: 238.1 [M+H]⁺

¹H NMR (500 MHz, DMSO-d₆) δ=12.84 (br s, 1H), 11.79 (br s, 1H), 6.98 (d, J=1.5 Hz, 1H), 6.68 (d, J=8.4 Hz, 1H), 6.48 (dd, J=1.7, 12.2 Hz, 1H), 4.74 (spt, J=6.0 Hz, 1H), 1.33 (d, J=6.1 Hz, 6H).

### Step 5. Synthesis of N-(1,1-dimethylsilinan-4-yl)-6-fluoro-4-isopropoxy-1H-indole-2-carboxamide

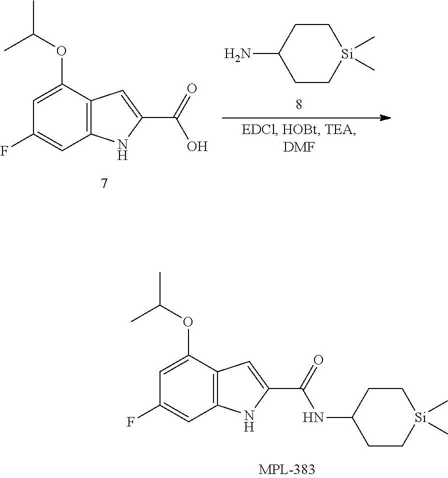

To a solution of 6-fluoro-4-isopropoxy-1H-indole-2-carboxylic acid (50 mg, 210.77 umol, 1 eq) and 1,1-dimethylsilinan-4-amine (45.47 mg, 252.92 umol, 1.2 eq, HCl salt) in DMF (2 mL) was added a solution of EDCI (121.21 mg, 632.31 umol, 3 eq) and HOBt (85.44 mg, 632.31 umol, 3 eq) in DMF (1 mL), followed by TEA (127.96 mg, 1.26 mmol, 176.02 uL, 6 eq). The mixture was stirred at 25° C. for 1 hr. LCMS showed desired mass. The reaction mixture was filtered to obtain filtrate, which was purified by prep-HPLC (column: YMC-Actus Triart C18 150*30 mm*5 um; mobile phase: A: 0.225% formic acid in water, B: CH₃CN, gradient: 62%-92% B over 11 min). Compound N-(1,1-dimethylsilinan-4-yl)-6-fluoro-4-isopropoxy-1H-indole-2-carboxamide (47.1 mg, 129.93 umol, 61.64% yield, 100% purity) was obtained as a white solid.

LCMS (ESI) m/z: 363.1 [M+H]⁺

¹H NMR (500 MHz, DMSO-d₆) δ=11.52 (s, 1H), 8.19 (d, J=8.2 Hz, 1H), 7.19 (d, J=1.8 Hz, 1H), 6.68 (dd, J=1.4, 9.5 Hz, 1H), 6.44 (dd, J=1.8, 12.3 Hz, 1H), 4.75 (spt, J=6.0 Hz, 1H), 3.73-3.61 (m, 1H), 2.01-1.90 (m, 2H), 1.63-1.51 (m, 2H), 1.33 (d, J=6.0 Hz, 6H), 0.76 (br d, J=14.6 Hz, 2H), 0.59 (dt, J=4.7, 14.2 Hz, 2H), 0.12-−0.02 (m, 6H).

### Example 27, MPL-384

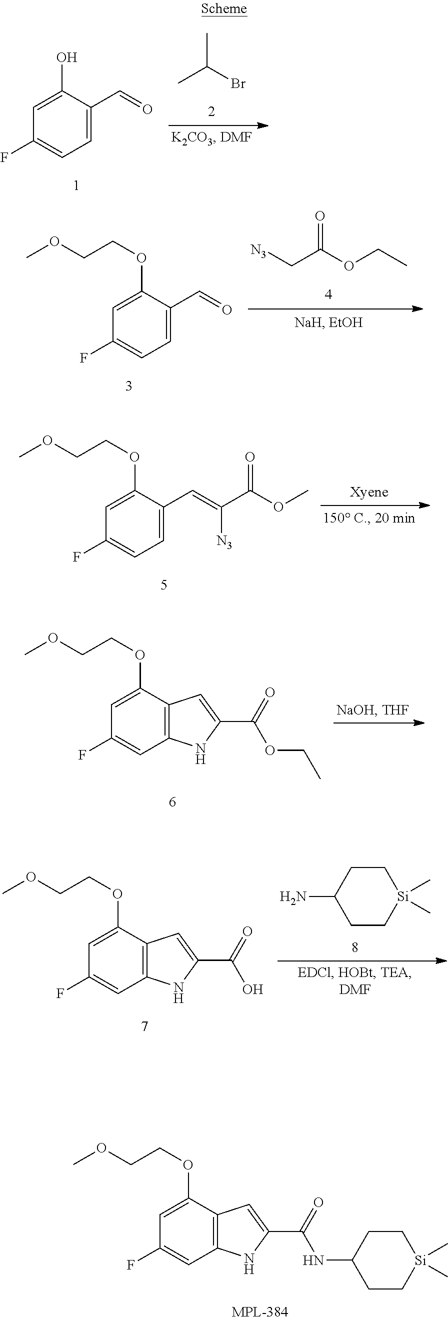

### Step 1. Synthesis of 4-fluoro-2-(2-methoxyethoxy)benzaldehyde

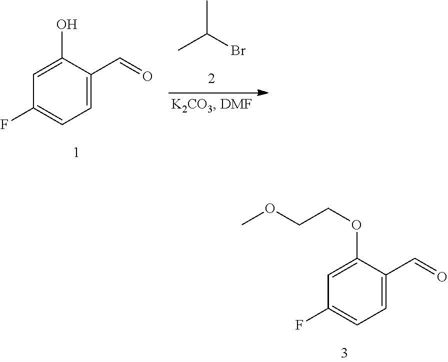

To a solution 4-fluoro-2-hydroxy-benzaldehyde (0.5 g, 3.57 mmol, 1 eq) in DMF (6 mL) was added K₂CO₃ (1.48 g, 10.71 mmol, 3 eq), followed by 1-bromo-2-methoxy-ethane (744.00 mg, 5.35 mmol, 502.70 uL, 1.5 eq). The mixture was stirred at 60° C. for 24 hr. TLC (Petroeluem ether:EtOAc=5:1) indicated reactant 1 was consumed completely and one new spot formed. The mixture was poured into water (50 mL) and extracted by EtOAc (20 mL×3). The combined organic layer was washed with LiCl (3% in water, 20 mL×2), dried with Na₂SO₄, filtered and concentrated under reduced pressure to give a residue which was purified by flash silica gel chromatography (0-25% Ethyl acetate in petroleum ether). Compound 4-fluoro-2-(2-methoxyethoxy)benzaldehyde (695 mg, 3.33 mmol, 93.35% yield, 95% purity) was obtained as a colorless oil. ¹H NMR was recorded.

### Step 2. Synthesis of ethyl (Z)-2-azido-3-[4-fluoro-2-(2-methoxyethoxy)phenyl]prop-2-enoate

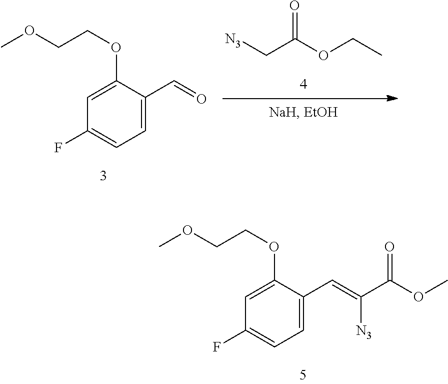

EtOH (10 mL) was placed in a well dried three-neck flask equipped with a thermometer purged with N₂, and NaH (701.28 mg, 17.53 mmol, 60% purity, 5 eq) was added in batches. The mixture was stirred until a clear solution was formed. The mixture was then cooled to −10° C., a solution of 4-fluoro-2-(2-methoxyethoxy)benzaldehyde (695 mg, 3.51 mmol, 1 eq) and ethyl 2-azidoacetate (2.26 g, 17.53 mmol, 2.46 mL, 5 eq) in THE (2 mL) was added dropwise at temperature below 0° C. The reaction solution was stirred at 0° C. for 2 hr. TLC (Petroleum ether:EtOAc=5:1) indicated one new spot formed. The mixture was poured into saturated NH₄Cl (30 mL), and then extracted with EtOAc (15 mL×2). The combined organic layer was dried by Na₂SO₄, and then filtered and concentrated under reduced pressure to give a residue which was purified by flash silica gel chromatography (0-15% Ethyl acetate in petroleum ether). Compound ethyl (Z)-2-azido-3-[4-fluoro-2-(2-methoxyethoxy)phenyl]prop-2-enoate (720 mg, 2.21 mmol, 63.06% yield, 95% purity) was obtained as a light yellow solid. ¹H NMR was recorded.

### Step 3. Synthesis of ethyl 6-fluoro-4-(2-methoxyethoxy)-1H-indole-2-carboxylate

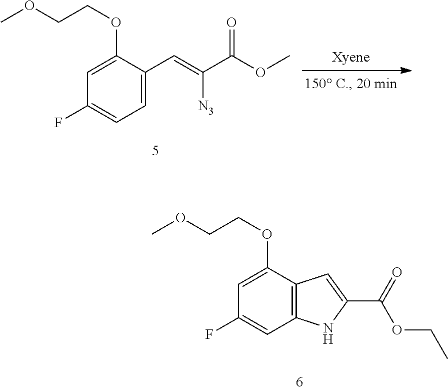

A solution of ethyl (Z)-2-azido-3-[4-fluoro-2-(2-methoxyethoxy)phenyl]prop-2-enoate (720 mg, 2.33 mmol, 1 eq) in xlyene (10 mL) was heated at 150° C. for 20 min. TLC (Petroleum ether:EtOAc=5:1) showed the starting material was consumed completely and one new spot formed. The reaction mixture was cooled to 20° C. gradually, and then filtered. The filter cake was washed with xlyene (5 mL×2) and collected. Compound ethyl 6-fluoro-4-(2-methoxyethoxy)-1H-indole-2-carboxylate (400 mg, 1.35 mmol, 58.03% yield, 95% purity) was obtained as a white solid. ¹H NMR was recorded.

### Step 4. Synthesis of 6-fluoro-4-(2-methoxyethoxy)-1H-indole-2-carboxylic acid

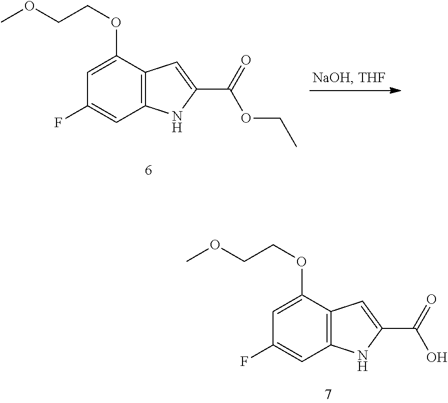

To a solution of ethyl 6-fluoro-4-(2-methoxyethoxy)-1H-indole-2-carboxylate (400 mg, 1.42 mmol, 1 eq) in EtOH (3 mL) was added NaOH (3 M, 2.85 mL, 6.01 eq) (aq.). The mixture was stirred at 80° C. for 2 hr. TLC (Petroleum ether:EtOAc=5:1) showed starting material was consumed completely and one new spot formed. The mixture was concentrated under reduced pressure to remove EtOH, and then HCl (3N in water) was added to adjust pH to 4. The product was collected by filtration. Compound 6-fluoro-4-(2-methoxyethoxy)-1H-indole-2-carboxylic acid (290 mg, 1.09 mmol, 76.51% yield, 95% purity) was obtained as a white solid.

¹H NMR (400 MHz, DMSO-d₆)=12.90 (br s, 1H), 11.85 (br s, 1H), 7.01 (d, J=1.5 Hz, 1H), 6.73 (d, J=9.5 Hz, 1H), 6.50 (dd, J=1.9, 12.1 Hz, 1H), 4.29-4.19 (m, 2H), 3.74 (dd, J=3.7, 5.3 Hz, 2H), 3.36 (s, 3H).

### Step 5. Synthesis of N-(1,1-dimethylsilinan-4-yl)-6-fluoro-4-(2-methoxyethoxy)-1H-indole-2-carboxamide

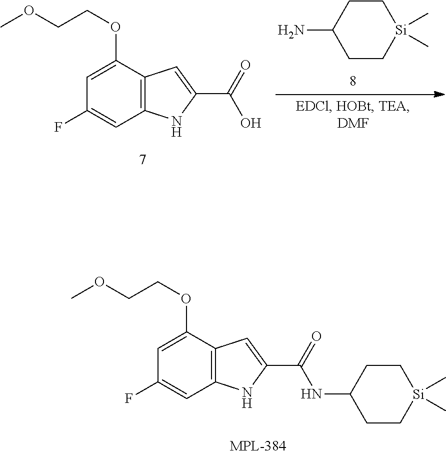

To a solution of 6-fluoro-4-(2-methoxyethoxy)-1H-indole-2-carboxylic acid (70 mg, 276.43 umol, 1 eq) and 1,1-dimethylsilinan-4-amine (59.63 mg, 331.72 umol, 1.2 eq, HCl) in DMF (1 mL) was added a solution of EDCI (105.99 mg, 552.87 umol, 2 eq) and HOBt (74.70 mg, 552.87 umol, 2 eq) in DMF (1 mL), followed by TEA (111.89 mg, 1.11 mmol, 153.90 uL, 4 eq). The mixture was stirred at 20° C. for 2 hr. LCMS showed reactant 7 was consumed completely and one main peak with desired mass was detected. The mixture was filtered to remove insoluble matter. The filtrate was purified by prep-HPLC (column: Phenomenex luna C18 100*40 mm*3 um; mobile phase: A: 0.225% formic acid in water, B: CH₃CN, gradient: 52%-82% B over 11 min). Compound N-(1,1-dimethylsilinan-4-yl)-6-fluoro-4-(2-methoxyethoxy)-1H-indole-2-carboxamide (71.3 mg, 187.54 umol, 67.84% yield, 99.56% purity) was obtained as a white solid.

LCMS (ESI) m/z 379.3 [M+H]⁺

¹H NMR (400 MHz, DMSO-d₆)=11.48 (d, J=1.5 Hz, 1H), 8.14 (d, J=8.3 Hz, 1H), 7.15 (d, J=1.8 Hz, 1H), 6.63 (dd, J=1.4, 9.6 Hz, 1H), 6.37 (dd, J=1.9, 12.1 Hz, 1H), 4.17-4.08 (m, 2H), 3.67-3.55 (m, 3H), 3.26 (br s, 3H), 1.94-1.82 (m, 2H), 1.56-1.42 (m, 2H), 0.68 (br d, J=14.5 Hz, 2H), 0.51 (dt, J=4.8, 14.1 Hz, 2H), 0.02-−0.08 (m, 6H).

### Example 28, MPL-399, MPL-399A and MPL-399B

### Synthesis of 4,6-dichloro-N-(1,1-dimethylsilolan-3-yl)-1H-indole-2-carboxamide; (R)-4,6-dichloro-N-(1,1-dimethylsilolan-3-yl)-1H-indole-2-carboxamide and (S)-4,6-dichloro-N-(1,1-dimethylsilolan-3-yl)-1H-indole-2-carboxamide

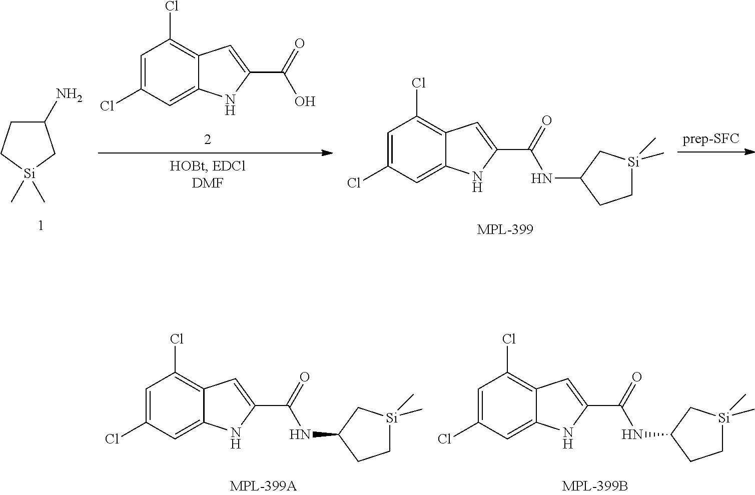

To a solution of 4,6-dichloro-1H-indole-2-carboxylic acid (30 mg, 130.41 umol, 1 eq) and 1,1-dimethylsilolan-3-amine (23.77 mg, 143.45 umol, 1.1 eq, HCl salt) in DMF (1 mL) was added a solution of EDCI (50.00 mg, 260.82 umol, 2 eq) and HOBt (35.24 mg, 260.82 umol, 2 eq) in DMF (1 mL), followed by TEA (52.78 mg, 521.63 umol, 72.60 uL, 4 eq). The mixture was stirred at 20° C. for 2 hr. LC-MS showed one main peak with desired mass. The mixture was diluted with MeOH (2 mL) and filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 70%-100% B over 11 min). Compound 4,6-dichloro-N-(1,1-dimethylsilolan-3-yl)-1H-indole-2-carboxamide (24.3 mg, 69.92 umol, 53.62% yield, 98.213% purity) was obtained as a white solid.

LCMS (ESI) m/z 340.9 [M+H]⁺

¹H NMR (400 MHz, DMSO-d₆) δ=11.86 (br s, 1H), 8.33 (d, J=7.6 Hz, 1H), 7.23 (d, J=0.7 Hz, 1H), 7.11 (s, 1H), 7.04 (d, J=1.7 Hz, 1H), 3.93-3.79 (m, 1H), 1.90-1.80 (m, 1H), 1.26 (dq, J=7.2, 12.2 Hz, 1H), 0.98-0.89 (m, 1H), 0.69-0.58 (m, 1H), 0.48 (dd, J=11.4, 14.1 Hz, 1H), 0.41-0.29 (m, 1H), 0.00 (s, 6H).

The same reaction was conducted later at 347.75 umol scale. The racemic MPL-399 was further purified by SFC (Berger MG II, column: Phenomenex-Cellulose-2 (250 mm*30 mm, 5 um); mobile phase: A: 0.1% NH₃H₂O in EtOH; B CO₂, gradient 15% B, isocratic, flow rate: 60 mL/min) to afford two peaks (two enantiomers), (R)-4,6-dichloro-N-(1,1-dimethylsilolan-3-yl)-1H-indole-2-carboxamide and (S)-4,6-dichloro-N-(1,1-dimethylsilolan-3-yl)-1H-indole-2-carboxamide.

Peak1 (MPL-399A): 30.2 mg, 88.48 umol, 25.44% yield, 100% purity.

LCMS (ESI) m/z 341.1 [M+H]⁺

¹H NMR (500 MHz, DMSO-d₆) δ=12.03 (br s, 1H), 8.50 (d, J=7.8 Hz, 1H), 7.44-7.38 (m, 1H), 7.29 (s, 1H), 7.22 (d, J=1.7 Hz, 1H), 4.10-3.97 (m, 1H), 2.09-1.99 (m, 1H), 1.44 (dq, J=7.1, 12.1 Hz, 1H), 1.16-1.06 (m, 1H), 0.86-0.76 (m, 1H), 0.66 (dd, J=11.3, 14.2 Hz, 1H), 0.59-0.47 (m, 1H), 0.18 (s, 6H).

Peak 2 (MPL-399B): 31.8 mg, 91.31 umol, 26.26% yield, 98% purity.

LCMS (ESI) m/z 341.1 [M+H]+; ¹H NMR (500 MHz, DMSO-d6) δ=12.03 (s, 1H), 8.50 (d, J=7.8 Hz, 1H), 7.41 (d, J=0.8 Hz, 1H), 7.29 (d, J=1.5 Hz, 1H), 7.22 (d, J=1.7 Hz, 1H), 4.10-3.97 (m, 1H), 2.09-1.99 (m, 1H), 1.44 (dq, J=7.2, 12.2 Hz, 1H), 1.17-1.06 (m, 1H), 0.81 (dd, J=6.5, 14.0 Hz, 1H), 0.66 (dd, J=11.3, 14.2 Hz, 1H), 0.53 (ddd, J=7.9, 12.7, 14.5 Hz, 1H), 0.22-0.13 (m, 6H).

MPL-399A and MPL-399B were also analyzed by analytical SFC.

Conditions:

Instrument: Waters UPCC with PDA Detector

Column: Cellulose 2 150 mm×4.6 mm, 5 um particle size.

Mobile phase: A: CO₂, B: 0.05% DEA in IPA

Gradient: 5% to 40% of B in 5 min and hold 40% B for 2.5 min, then 5% B for 2.5 min.

Flow rate: 2.5 mL/min.

Column temp.: 35° C.

ABPR: 1500 psi

MPL-399A: retention time, 4.13 min; 100% ee; MPL-399B: retention time, 4.35 min; 89.4% ee

### Example 29, MPL-400

### Synthesis of N-(1,1-dimethylsilolan-3-yl)-4,6-difluoro-1H-indole-2-carboxamide

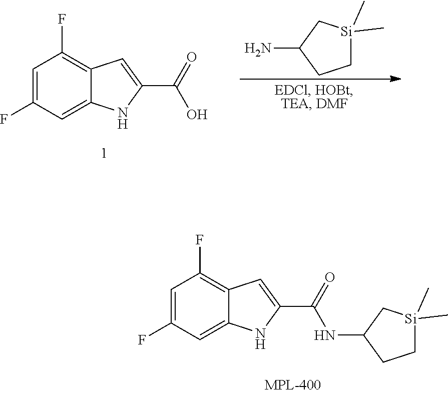

To a solution of 4,6-difluoro-1H-indole-2-carboxylic acid (30 mg, 152.18 umol, 1 eq) and 1,1-dimethylsilolan-3-amine (27.74 mg, 167.40 umol, 1.1 eq, HCl salt) in DMF (1 mL) was added a solution of EDCI (58.34 mg, 304.36 umol, 2 eq) and HOBt (41.12 mg, 304.36 umol, 2 eq) in DMF (1 mL), followed by TEA (61.60 mg, 608.71 umol, 84.73 uL, 4 eq). The mixture was stirred at 20° C. for 2 hr. LC-MS showed reactant 1 was consumed completely and one main peak with desired mass was detected. The mixture was diluted with MeOH (2 mL) and filtered to remove insoluble matter. The filtrate was purified by prep-HPLC (column: YMC-Actus Triart C18 150*30 mm*5 um; mobile phase: A: 0.225% formic acid in water, B: CH₃CN, gradient: 62%-92% B over 11 min). Compound N-(1,1-dimethylsilolan-3-yl)-4,6-difluoro-1H-indole-2-carboxamide (33.2 mg, 106.75 umol, 70.15% yield, 99.158% purity) was obtained as a white solid.

LCMS (ESI) m/z 309.0 [M+H]⁺

¹H NMR (400 MHz, DMSO-d₆) δ=11.76 (br s, 1H), 8.17 (br d, J=7.6 Hz, 1H), 7.08 (d, J=1.2 Hz, 1H), 6.84 (dd, J=1.3, 9.4 Hz, 1H), 6.69 (dt, J=2.1, 10.5 Hz, 1H), 3.85 (dq, J=6.6, 11.7 Hz, 1H), 1.92-1.81 (m, 1H), 1.26 (dq, J=7.2, 12.1 Hz, 1H), 0.98-0.87 (m, 1H), 0.68-0.58 (m, 1H), 0.47 (dd, J=11.2, 14.2 Hz, 1H), 0.41-0.30 (m, 1H), 0.00 (d, J=1.0 Hz, 6H).

### Example 30, MPL-385

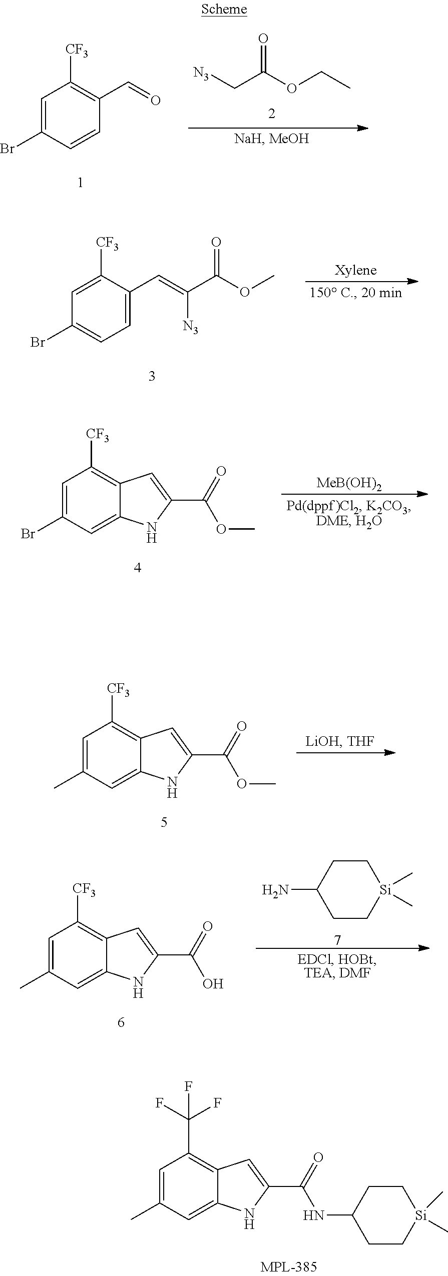

### Step 1. Synthesis of methyl (Z)-2-azido-3-[4-bromo-2-(trifluoromethyl)phenyl]prop-2-enoate

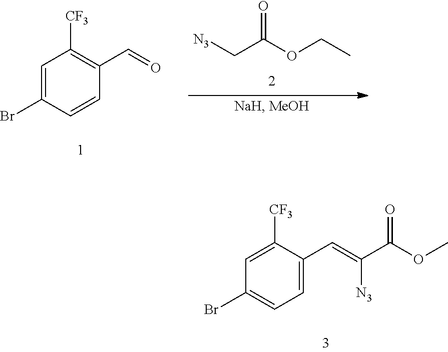

NaH (3.95 g, 98.81 mmol, 60% purity, 5 eq) was added to a solution of MeOH (30 mL) at 0° C. The reaction mixture was stirred at 0° C. for 0.5 hr. Then a solution of 4-bromo-2-(trifluoromethyl) benzaldehyde (5 g, 19.76 mmol, 1 eq) and ethyl 2-azidoacetate (12.76 g, 98.81 mmol, 13.87 mL, 5 eq) in THE (20 mL) was added. The reaction mixture was stirred at 0° C. for 1 hr. TLC showed one major new spot with lower polarity. The reaction mixture was quenched with saturated NH₄Cl (50) mL at 0° C., and then extracted with EtOAc (50 mL×2). The combined organic layer was dried over Na₂SO₄, and then filtered and concentrated under reduced pressure. The residue was purified by flash silica gel chromatography (0-30% Ethyl acetate in petroleum ether). Compound methyl (Z)-2-azido-3-[4-bromo-2-(trifluoromethyl)phenyl]prop-2-enoate (6.9 g, crude) was obtained as a yellow oil.

### Step 2. Synthesis of methyl 6-bromo-4-(trifluoromethyl)-1H-indole-2-carboxylate

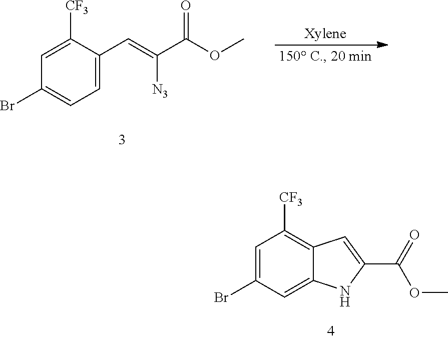 [SMILES: COC(=O)/C(=C/c1ccc(Br)cc1C(F)(F)F)N=[N+]=[N-] ; "150° C., 20 min" ; "Xylene" ; COC(=O)c1cc2c(C(F)(F)F)cc(Br)cc2[nH]1]

A mixture of methyl (Z)-2-azido-3-[4-bromo-2-(trifluoromethyl)phenyl]prop-2-enoate (6.9 g, 19.71 mmol, 1 eq) in xylene (20 mL) was degassed and purged with N₂ for 3 times, and then stirred at 150° C. for 1 hr under N₂ atmosphere. TLC showed one major new spot with higher polarity. The reaction mixture was cooled to room temperature and filtered. The filtrate was purified by flash silica gel chromatography (0-20% Ethyl acetate in petroleum ether). Compound methyl 6-bromo-4-(trifluoromethyl)-1H-indole-2-carboxylate (230 mg, 678.41 umol, 3.44% yield, 95% purity) was obtained as a yellow solid. ¹H NMR was recorded.

### Step 3. Synthesis of methyl 6-methyl-4-(trifluoromethyl)-1H-indole-2-carboxylate

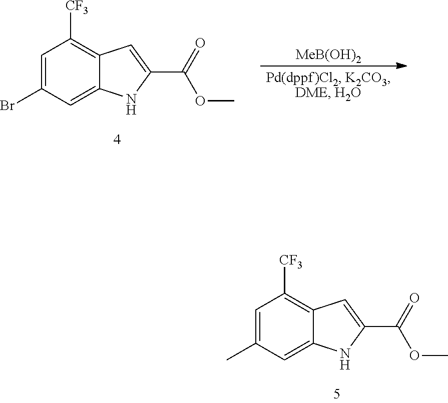

To a solution of methyl 6-bromo-4-(trifluoromethyl)-1H-indole-2-carboxylate (230 mg, 714.11 umol, 1 eq) and methylboronic acid (128.24 mg, 2.14 mmol, 3 eq) in dioxane (10 mL) and H₂O (0.1 mL) was added Pd(dppf)Cl₂ (104.50 mg, 142.82 umol, 0.2 eq) and K₂CO₃ (296.09 mg, 2.14 mmol, 3 eq). The mixture was stirred at 110° C. for 12 hr under N₂. LC-MS indicated desired mass was detected. The reaction mixture was concentrated under reduced pressure. The residue was purified by flash silica gel chromatography (0-30% Ethyl acetate in petroleum ether). Compound methyl 6-methyl-4-(trifluoromethyl)-1H-indole-2-carboxylate (120 mg, 443.22 umol, 62.07% yield, 95% purity) was obtained as a white solid.

LCMS (ESI) m/z: 258.0 [M+H]⁺

¹H NMR (500 MHz, METHANOL-d4) δ=7.51 (s, 1H), 7.30 (s, 1H), 7.20 (d, J=1.5 Hz, 1H), 3.96 (s, 3H).

### Step 4. Synthesis of 6-methyl-4-(trifluoromethyl)-1H-indole-2-carboxylic acid

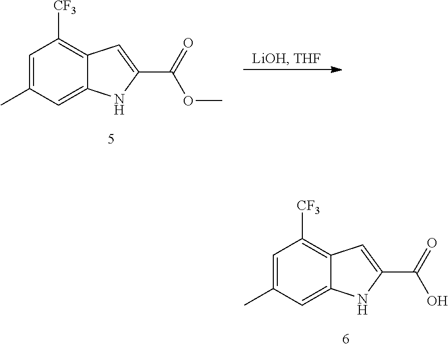

To a solution of methyl 6-methyl-4-(trifluoromethyl)-1H-indole-2-carboxylate (120 mg, 466.55 umol, 1 eq) in THE (3 mL) was added a solution of LiOH.H₂O (156.62 mg, 3.73 mmol, 8 eq) in H₂O (3 mL). The mixture was stirred at 30° C. for 5 hr. LC-MS indicated desired mass was detected. The reaction mixture was diluted with 20 ml of H₂O and adjusted pH to 4 with 3 M HCl in water, and then extracted with EtOAc mL (30 mL×2). The combined organic layer was dried over Na₂SO₄, and then filtered and concentrated under reduced pressure. Compound 6-methyl-4-(trifluoromethyl)-1H-indole-2-carboxylic acid (90 mg, 296.08 umol, 63.46% yield, 80% purity) was obtained as a yellow solid. The crude product was used for the next step without purification.

LCMS (ESI) m/z: 244.1 [M+H]⁺

### Step 5. Synthesis of N-(1,1-dimethylsilinan-4-yl)-6-methyl-4-(trifluoromethyl)-1H-indole-2-carboxamide

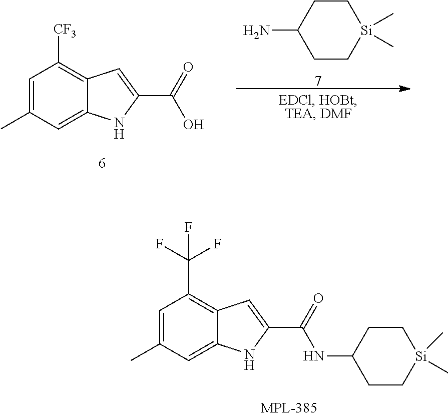

To a solution of 6-methyl-4-(trifluoromethyl)-1H-indole-2-carboxylic acid (90 mg, 370.09 umol, 1 eq), 1,1-dimethylsilinan-4-amine (66.53 mg, 370.09 umol, 1 eq, HCl salt) in DMF (2 mL) was added HOBt (150.02 mg, 1.11 mmol, 3 eq) and EDCI (212.84 mg, 1.11 mmol, 3 eq) and TEA (224.70 mg, 2.22 mmol, 309.07 uL, 6 eq). The mixture was stirred at 25° C. for 16 hr. LC-MS indicated desired mass was detected. The reaction mixture was purified by prep-HPLC (column: Phenomenex Synergi C18 150×30 mm×4 um; mobile phase: A: 0.05% HCl in water, B: CH₃CN; gradient: 69%-89% B over 9 min). Compound N-(1,1-dimethylsilinan-4-yl)-6-methyl-4-(trifluoromethyl)-1H-indole-2-carboxamide (23.1 mg, 61.30 umol, 16.56% yield, 97.78% purity) was obtained as a white solid.

LCMS (ESI) m/z: 369.1 [M+H]⁺

¹H NMR (400 MHz, METHANOL-d₄) δ=7.43 (s, 1H), 7.19 (s, 2H), 3.74 (br t, J=11.5 Hz, 1H), 2.14-2.02 (m, 2H), 1.71-1.55 (m, 2H), 0.86-0.74 (m, 2H), 0.72-0.59 (m, 2H), 0.08 (s, 3H), 0.00 (s, 3H).

### Example 31, MPL-386

### Synthesis of N-(1,1-dimethylsilocan-5-yl)-6-methyl-4-(trifluoromethyl)-1H-indole-2-carboxamide

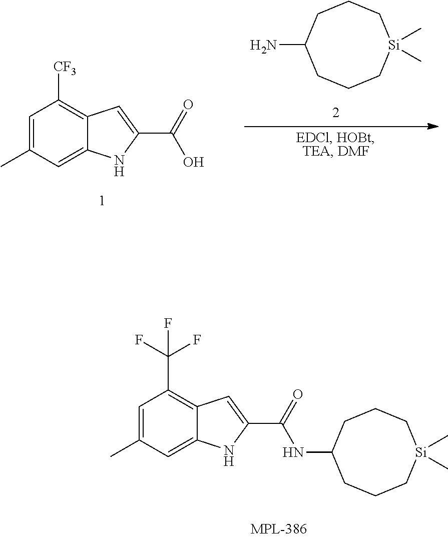

To a solution of 6-methyl-4-(trifluoromethyl)-1H-indole-2-carboxylic acid (50 mg, 205.61 umol, 1.42 eq), 1,1-dimethylsilocan-5-amine (30 mg, 144.36 umol, 1 eq, HCl salt) in DMF (2 mL) was added HOBt (58.52 mg, 433.08 umol, 3 eq) and EDCI (83.02 mg, 433.08 umol, 3 eq) and TEA (87.65 mg, 866.15 umol, 120.56 uL, 6 eq). The mixture was stirred at 25° C. for 1 hr. LC-MS indicated desired mass was detected. The reaction mixture was diluted with H₂O (30 mL) and extracted with EtOAc mL (30 mL×3). The combined organic layer was washed with saturated NaHCO₃ (30 mL×2) and 5% LiCl (30 mL×2), dried over Na₂SO₄, and then filtered and concentrated under reduced pressure. The residue was purified by prep-HPLC (column: Phenomenex Synergi C18 150×30 mm×4 um; mobile phase: A: 0.05% HCl in water, B: CH₃CN, Gradient: 78%-98% B over 9 min). The product isolated from prep-HPLC was further purified by SFC (Sepiatec Prep SFC 100, column: DAICEL CHIRALPAK AD-H (250 mm*30 mm, Sum); mobile phase: 0.1% NH₃H₂O in IPA, B: CO₂; 25% B isocratic; flowrate: 60 m/min). Compound N-(1,1-dimethylsilocan-5-yl)-6-methyl-4-(trifluoromethyl)-1H-indole-2-carboxamide (7.7 mg, 19.40 umol, 15.39% yield, 99.923% purity) was obtained as a white solid.

LCMS (ESI) m/z: 397.1 [M+H]⁺

¹H NMR (400 MHz, METHANOL-d₄) δ=7.49 (s, 1H), 7.24 (br s, 2H), 4.21 (br s, 1H), 2.49 (s, 3H), 1.90-1.66 (m, 8H), 0.82 (br s, 4H), 0.04 (d, J=6.3 Hz, 6H).

### Example 32, MPL-470

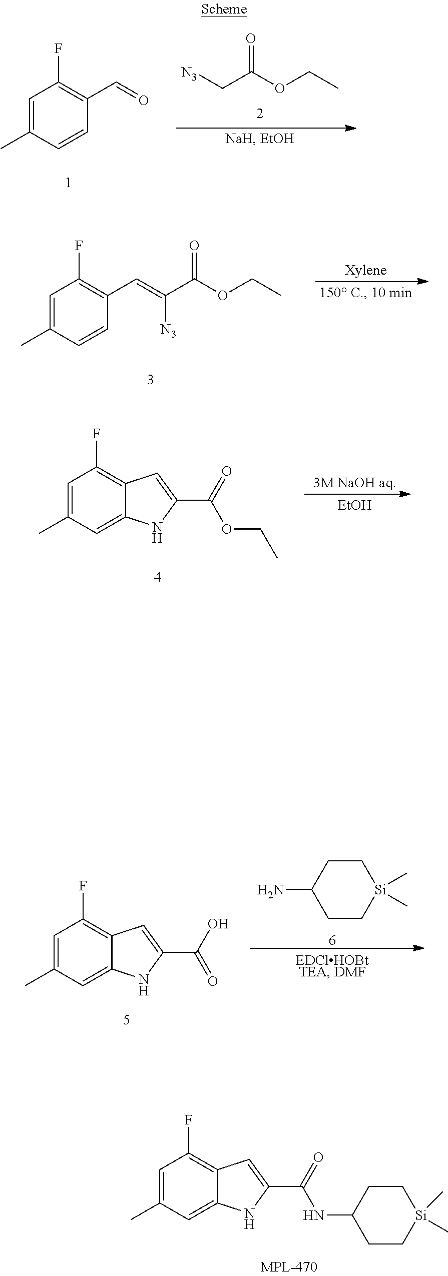

### Step 1. Synthesis of ethyl (Z)-2-azido-3-(2-fluoro-4-methyl-phenyl) prop-2-enoate

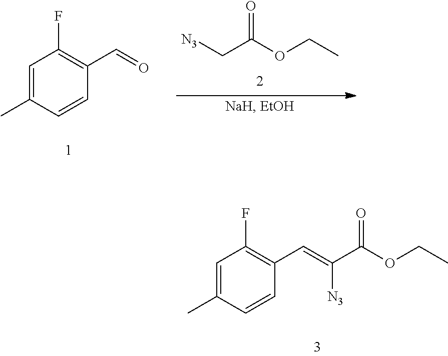

NaH (434.30 mg, 10.86 mmol, 60% purity, 5 eq) was added to EtOH (8 mL) in batches. The mixture was stirred until a clear solution formed, and then cooled to −10° C. A solution of 2-fluoro-4-methyl-benzaldehyde (300 mg, 2.17 mmol, 1 eq) and ethyl 2-azidoacetate (1.40 g, 10.86 mmol, 1.52 mL, 5 eq) in EtOH (2 mL) was added dropwise at the temperature below 0° C., the mixture was stirred at −10° C.˜0° C. for 3 hr. TLC (Petroleum ether:EtOAc=5:1) showed starting material was consumed completely, one new spot formed. The mixture was poured into saturated NH₄Cl (30 mL), and then extracted by EtOAc (25 mL). The organic layer was washed with brine (10 mL), dried by Na₂SO₄, and then filtered and concentrated under reduced pressure. The residue was purified by flash silica gel chromatography (0-10% Ethyl acetate in petroleum ether). Compound ethyl (Z)-2-azido-3-(2-fluoro-4-methyl-phenyl)prop-2-enoate (400 mg, 1.52 mmol, 70.20% yield, 95% purity) was obtained as a yellow oil. ¹H NMR was recorded.

### Step 2. Synthesis of ethyl 4-fluoro-6-methyl-1H-indole-2-carboxylate

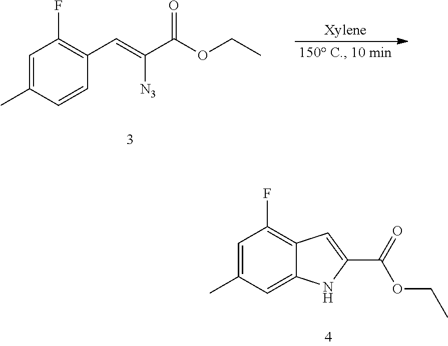 [SMILES: CCOC(=O)/C(=C/c1ccc(C)cc1F)N=[N+]=[N-] ; "150° C., 10 min" ; "Xylene" ; CCOC(=O)c1cc2c(F)cc(C)cc2[nH]1]

A solution of ethyl (Z)-2-azido-3-(2-fluoro-4-methyl-phenyl) prop-2-enoate (700 mg, 2.81 mmol, 1 eq) in xylene (14 mL) was stirred at 150° C. for 10 min. TLC (Petroleum ether:EtOAc=10:1) indicated reactant 3 was consumed completely, several new spots formed. The solution was cooled to 25° C., there were precipitates after 5 hr which was collected by filtration. The cake was washed with petroleum ether (5 mL), and then purified by flash silica gel chromatography (0-10% Ethyl acetate in petroleum ether). Compound ethyl 4-fluoro-6-methyl-1H-indole-2-carboxylate (152 mg, 652.72 umol, 23.24% yield, 95% purity) was obtained as a yellow solid. ¹H NMR was collected.

### Step 3. Synthesis of 4-fluoro-6-methyl-1H-indole-2-carboxylic acid

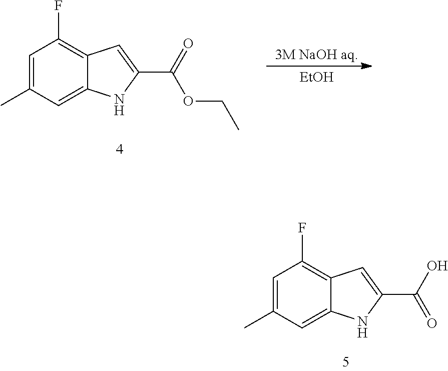 [SMILES: "EtOH" ; CCOC(=O)c1cc2c(F)cc(C)cc2[nH]1 ; "3M NaOH aq." ; Cc1cc(F)c2cc(C(=O)O)[nH]c2c1]

To a solution of ethyl 4-fluoro-6-methyl-1H-indole-2-carboxylate (150 mg, 678.04 umol, 1 eq) in EtOH (7.5 mL) was added NaOH (3 M in water, 7.5 mL, 33.18 eq). The mixture was stirred at 80° C. for 2 hr. TLC (Petroleum ether:EtOAc=10:1) indicated reactant 4 was consumed completely, and one major new spot with higher polarity formed. EtOH was removed under reduced pressure. The resulting mixture was acidified to pH around 5 with HCl (6 N in water). The suspension was collected by filtration. The cake was washed with water (5 mL) and dried by lyophilizer. Compound 4-fluoro-6-methyl-1H-indole-2-carboxylic acid (90 mg, 442.61 umol, 65.28% yield, 95% purity) was obtained as a white solid.

¹H NMR (400 MHz, DMSO-d₆) δ=11.99-11.89 (m, 1H), 7.07-7.01 (m, 2H), 6.70 (d, J=11.3 Hz, 1H), 2.39 (s, 3H).

### Step 4: Synthesis of N-(1,1-dimethylsilinan-4-yl)-4-fluoro-6-methyl-1H-indole-2-carboxamide

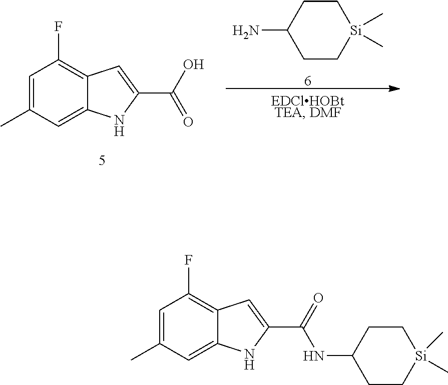

To a solution of 4-fluoro-6-methyl-1H-indole-2-carboxylic acid (45 mg, 232.95 umol, 1 eq) and 1,1-dimethylsilinan-4-amine (50.25 mg, 279.54 umol, 1.2 eq, HCl salt) in DMF (1 mL) was added a solution of HOBt (62.95 mg, 465.90 umol, 2 eq) and EDCI (89.31 mg, 465.90 umol, 2 eq) in DMF (1 mL), followed by TEA (94.29 mg, 931.80 umol, 129.69 uL, 4 eq). The mixture was stirred at 25° C. for 2 hr. LC-MS showed reactant 5 was consumed completely and one main peak with desired mass was detected. The mixture was filtered. The filtrate was purified by prep-HPLC (Phenomenex luna C18 100*40 mm*3 um; mobile phase: A: 0.225% formic acid in water, B: CH₃CN; gradient: 65%-95% B over 15 min). Compound N-(1,1-dimethylsilinan-4-yl)-4-fluoro-6-methyl-1H-indole-2-carboxamide (55.6 mg, 174.59 umol, 74.95% yield, 100% purity) was obtained as a white solid.

LCMS (ESI) m/z 319.3 [M+H]⁺

¹H NMR (400 MHz, DMSO-d₆) δ=11.69 (br s, 1H), 8.24 (d, J=8.1 Hz, 1H), 7.17 (d, J=1.5 Hz, 1H), 7.04 (s, 1H), 6.65 (d, J=11.4 Hz, 1H), 3.78-3.65 (m, 1H), 2.37 (s, 3H), 2.05-1.92 (m, 2H), 1.66-1.52 (m, 2H), 0.83-0.72 (m, 2H), 0.61 (dt, J=4.8, 14.1 Hz, 2H), 0.12-0.00 (m, 6H).

### Example 33, MPL-256

### Synthesis of 4,6-difluoro-N-(6-silaspiro[5.5]undecan-3-yl)-1H-indole-2-carboxamide

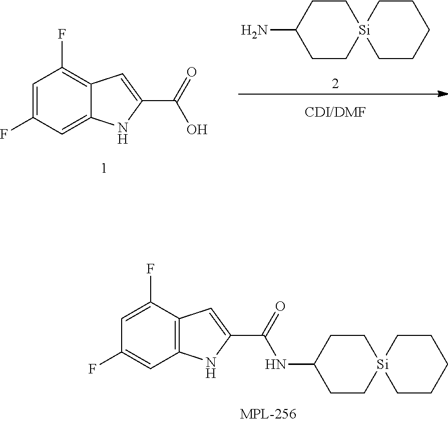

To a solution of 4, 6-difluoro-1H-indole-2-carboxylic acid (50 mg, 253.63 umol, 1 eq) in DMF (1.5 mL) was added CDI (49.35 mg, 304.36 umol, 1.2 eq). The mixture was stirred at 30° C. for 0.5 h. Then 6-silaspiro[5.5]undecan-3-amine (55.81 mg, 304.36 umol, 1.2 eq) was added. The mixture was stirred at 30° C. for 11.5 h. TLC (Petroleum ether: Ethyl acetate=5:1) showed there was a little starting material. The reaction was added dropwise to H₂O (20 mL). The precipitates were collected by filtration and was purified by column chromatography (SiO₂, Petroleum ether: Ethyl acetate=5:1). Compound 4,6-difluoro-N-(6-silaspiro[5.5]undecan-3-yl)-1H-indole-2-carboxamide (47 mg, 128.08 umol, 50.50% yield, 98.781% purity) was obtained as a white solid.

LCMS (ESI) m/z 363.1[M+H]⁺

¹H NMR (500 MHz, DMSO-d6) δ=11.94 (br s, 1H), 8.30 (br d, J=8.1 Hz, 1H), 7.25 (s, 1H), 7.01 (br d, J=9.0 Hz, 1H), 6.87 (br t, J=10.2 Hz, 1H), 3.76-3.67 (m, 1H), 2.00 (br d, J=8.9 Hz, 2H), 1.70-1.51 (m, 6H), 1.38 (br s, 2H), 0.90 (br d, J=14.6 Hz, 2H), 0.72-0.67 (m, 2H), 0.63-0.54 (m, 4H).

### Example 34, MPL-257

### Synthesis of 4,6-dichloro-N-(6-silaspiro[5.5]undecan-3-yl)-1H-indole-2-carboxamide

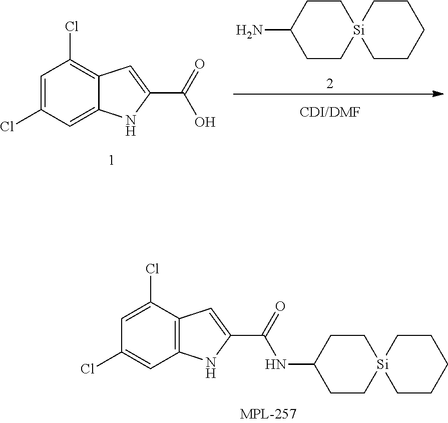

To a solution of 4, 6-dichloro-1H-indole-2-carboxylic acid (50 mg, 217.35 umol, 1 eq) in DMF (1.5 mL) was added CDI (42.29 mg, 260.82 umol, 1.2 eq). The mixture was stirred at 30° C. for 0.5 h. Then 6-silaspiro[5.5]undecan-3-amine (47.82 mg, 260.82 umol, 1.2 eq) was added. The mixture was stirred at 30° C. for 11.5 h. TLC (Petroleum ether:EtOAc=1:1, R_{f}=0.5) showed there were a little starting material and one major new spot with higher polarity. The reaction was added dropwise to H₂O (20 mL). The precipitates were collected by filtration and then purified by column chromatography (SiO₂, Petroleum ether: Ethyl acetate=1:0 to 1:1). Compound 4,6-dichloro-N-(6-silaspiro[5.5]undecan-3-yl)-1H-indole-2-carboxamide (10 mg, 24.91 umol, 11.46% yield, 98.49% purity) was obtained as a yellow solid.

LCMS (ESI) m/z 395.0 [M+H]⁺

¹H NMR (500 MHz, CHLOROFORM-d) δ=10.18 (br s, 1H), 7.41 (s, 1H), 7.16 (s, 1H), 6.86 (br s, 1H), 6.13 (br d, J=8.2 Hz, 1H), 3.95 (br d, J=9.6 Hz, 1H), 2.21 (br s, 2H), 1.68 (br d, J=6.4 Hz, 6H), 1.42 (br s, 2H), 0.93 (br d, J=14.6 Hz, 2H), 0.77-0.69 (m, 4H), 0.64 (br s, 2H).

### Example 35

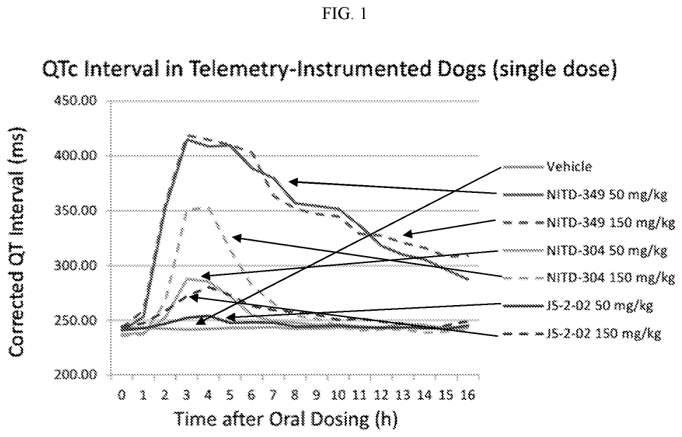

NITD-304 and NITD-349 showed potent activity against both drug-sensitive and multidrug-resistant clinical isolates of Mtb and were identified as lead candidates by Rao et al. (2013). However, applicants observed dramatic corrected QT (QTc) prolongation in dogs with both NITD-304 and NITD-349 even though each of their hERG IC₅₀ values were greater than 50 μM (Table 1 and FIG. 1). QTc prolongation is associated with cardiovascular disorders (Beinart 2014). It was discovered that both NITD-304 and NITD-349 inhibit the hKCNQ1 channel (Table 2). Applicants discovered that compounds with a silicon atom within the amine moiety, such as MPL-203 and MPL-204 depicted in Table 3, do not inhibit the hKCNQ1 channel.

TABLE 1

| | 50 mg/kg, PO | | 150 mg/kg, PO | |
|---|---|---|---|---|
| | | QTcV @ 1 hr | Max. | QTcV @ 1 hr |
| Compound | Max. QTcV (ms) | (C1 hr, μg/mL) | QTcv (ms) | (C1 hr, μg/mL) |
| NITD-349 | 170 | 11* (0.79) | 170 | 13.6 (0.94) |
| NITD-304 | 49 | −0.6 (0.25) | 120 | 0.3 (0.55) |
| JS-2-02 | 6.8 | 1.2 (0.57) | 37 | 2.8 ((1.1) |

*The QT prolongation may be due to multiple channel effects

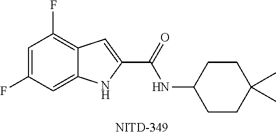 [SMILES: CC1(C)CCC(NC(=O)c2cc3c(F)cc(F)cc3[nH]2)CC1]

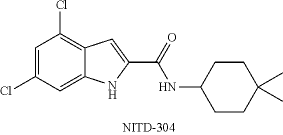 [SMILES: CC1(C)CCC(NC(=O)c2cc3c(Cl)cc(Cl)cc3[nH]2)CC1]

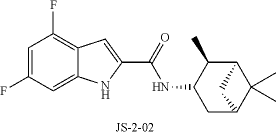

TABLE 2
In Vitro Assessment of Five Ion Channels

| | IC50, μM | | | | |
|---|---|---|---|---|---|
| Compounds | hKCNQ1/MinK | hERG | hNav1.5 Peak | hNav1.05 Late | hCav1.2 |
| Control | 5.18 | 3.2 | 0.20 | 2.1 | 0.0042 |
| NITD-349 | 0.058 | >10 | >10 | >10 | 9.53 |
| NITD-304 | 0.71 | >10 | >10 | 6.4 | 2.01 |
| JS-2-02 | 6.2 | >10 | 5.17 | >10 | >10 |

NITD-349 and NITD-304 showed potent hKCNQ1 inhibition.

TABLE 3
Effect of Cycloalkyl Substitution

| | | | IC50, μM | | | | |
|---|---|---|---|---|---|---|---|
| | | MIC | hKCNQ1/ | | hNav1.5 | hNav1.5 | |
| | | (μg/mL) | Mink | hERG | Peak | Late | hCav1.2 |
| NITD-349 | 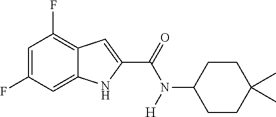 [SMILES: CC1(C)CCC(NC(=O)c2cc3c(F)cc(F)cc3[nH]2)CC1] | 0.005-0.04 | 0.058 | >10 | >10 | >10 | 9.5 |
| NITD-304 | 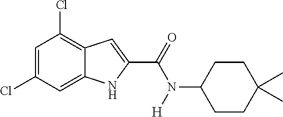 [SMILES: CC1(C)CCC(NC(=O)c2cc3c(Cl)cc(Cl)cc3[nH]2)CC1] | 0.005-0.04 | 0.71 | >10 | >10 | 6.4 | 2.01 |
| MPL-203 | 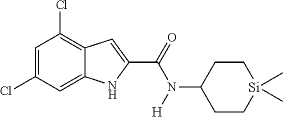 [SMILES: C[Si]1(C)CCC(NC(=O)c2cc3c(Cl)cc(Cl)cc3[nH]2)CC1] | 0.003 | 2.71 | >10.00 | >10.00 | >10.00 | >10.00 |
| MPL-204 | 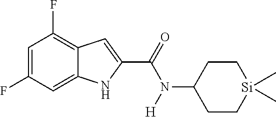 [SMILES: C[Si]1(C)CCC(NC(=O)c2cc3c(F)cc(F)cc3[nH]2)CC1] | 0.003 | 7.56 | >10.00 | >10.00 | >10.00 | >10.00 |

Dimethyl silacyclohexyl showed significant effect on cardiac channels.

### Example 36

MIC (Minimum Inhibitory Concentration) determination of anti-tuberculosis drugs. The antituberculosis activity of each compound against M. tb H37Rv was measured by the green fluorescent protein reporter assay (Collins 1998). Briefly, the compound was initially dissolved in dimethylsulfoxide (DMSO) and two fold dilutions were made in DMSO. The same amount of each dilution of compound solution was added to 7H9 broth in microplates. The initial inoculum of 2×10⁵ CFU/ml of Mtb H37Rv-GFP that was grown in Middlebrook 7H9 media was exposed to the compound for 10 days. The fluorescence was measured in a Fluostar Optima microplate fluorometer (BMG Labtech, Germany), and the MIC was defined as the lowest concentration of compounds that inhibited fluorescence by 90% comparing to the fluorescence of bacteria only wells. CFU=colony forming units. Columns 1 and 3 of Table 4 show anti-Mycobacterium tuberculosis activity of representative compounds of the invention. The procedures described in Collins 1997 were used to generate the data shown in column 1. The procedures described in Cho 2007 were used to generate the data shown in column 3.

The procedures described in Falzari 2005 were used to generate the data shown in column 2 of Table 4.

Column 5 of Table 4 shows anti-Mycobacterium abscessus activity of representative compounds of the invention. The procedure described in Franz 2017 was used to generate the data shown in column 5 of Table 4.

TABLE 4

| | 1 | 2 | 3 | 4 | 5 |
|---|---|---|---|---|---|
| | M. tb H37Rv: | VERO Cell: | M. tb H37Rv: | M. tb H37Rv: | M. ab_ATCC: |
| | MIC-MABA | Cytotoxicity- | MIC-LORA | MIC-LORA: | MIC MHII: |
| Compound | (μg/mL) | IC₅₀ (μg/mL) | (μg/mL) | Comment | MIC (μg/mL) |
| MPL-262 | 0.009 | | 16 | | 0.5 |
| MPL-265 | 0.014 | >20 | >20 | 84% inhibition | >64 |
| | | | | at 20 μg/mL | |
| MPL-264 | 0.011 | >20 | >20 | 85% inhibition | 64 |
| | | | | at 20 μg/mL | |
| MPL-286 | 0.0044 | >20 | 7.9 | | >64 |
| MPL-257 | 0.0045 | | >20 | 68% inhibition | >64 |
| | | | | at 20 μg/mL | |
| MPL-252 | 0.014 | >20 | | | 64 |
| MPL-256 | 0.0041 | >20 | 3.4 | | >64 |
| MPL-251 | 0.0043 | >20 | 7 | | >64 |
| MPL-204 | 0.0035 | >32 | >32 | | >64 |
| MPL-203 | 0.0033 | >32 | >32 | | >64 |
| MPL-196 | 0.0048 | >32 | >32 | | >64 |
| MPL-296 | 0.0151 | 13.8 | 2.3 | | 0.062 |
| MPL-296A | 0.0007- | >64 | ND | | <0.063 |
| MPL-296B | 0.052- | >64 | ND | | 0.5 |
| MPL-303 | <0.004 | >20 | 4.23 | | 4 |
| MPL-317 | 0.031 | >20 | >20 | | 0.062 |
| MPL-324 | 0.0154 | >20 | >20 | | >64 |
| MPL-325 | 0.0305 | 16.4 | >20 | | >64 |
| MPL-326 | 0.0305 | 15.4 | >20 | | >64 |
| MPL-327 | 0.00752 | >20 | >20 | | >64 |
| MPL-334 | <0.004 | >20 | >20 | | >64 |
| MPL-335 | <0.004 | >20 | >20 | | >64 |
| MPL-336 | 0.0306 | >20 | >20 | | >64 |
| MPL-337 | 0.007 | >20 | >20 | | >64 |
| MPL-338 | <0.004 | >20 | >20 | | >64 |
| MPL-339 | <0.004 | >20 | >20 | | >64 |
| MPL-340 | <0.004 | >20 | >20 | | >64 |
| MPL-344 | 0.008 | >20 | >20 | | >64 |
| MPL-360 | 87% inh.at 1 | 15.4 | >20 | | |
| | ug/mL | | | | |
| MPL-383 | 0.0148 | >20 | >20 | | >64 |
| MPL-384 | 0.0308 | >20 | 9.36 | | >64 |
| MPL-399 | <0.004 | >20 | 7.81 | | 0.062 |
| MPL-399A | 0.030 | >64 | ND | | 0.125 |
| MPL-399B | 0.003 | 52 | ND | | 0.125 |
| MPL-400 | 0.0113 | >20 | >20 | | >64 |
| MPL-385 | <0.004 | >20 | 12.7 | | 64 |
| MPL-386 | <0.004 | >20 | >20 | | >64 |
| MPL-470 | 0.007 | >20 | >20 | | >64 |

Key: MIC: Minimum Inhibitory Concentration;
MABA: microplate-based Alamar Blue assay;
LORA: low oxygen recovery assay;
Mab: Mycobacterium abscessus;
ATCC: American Type Culture Collection.
ND: Not determined.

In summary, the compounds of the invention exhibit potent anti-Mycobacterium activity (against Mycobacterium tuberculosis and non-tuberculosis Mycobacterium infections) and possess less untoward cardiovascular side effects exhibited by known compounds.

### REFERENCES

Beinart, Roy, et al. “The QT Interval Is Associated with Incident Cardiovascular Events in the Multi-Ethnic Study of Atherosclerosis.” Journal of the American College of Cardiology, vol. 64, no. 20, November 2014, pp. 2111-19. PubMed Central, doi:10.1016/j.jacc.2014.08.039.
Cho, Sang Hyun, et al. “Low-Oxygen-Recovery Assay for High-Throughput Screening of Compounds against Nonreplicating Mycobacterium Tuberculosis.” Antimicrobial Agents and Chemotherapy, vol. 51, no. 4, April 2007, pp. 1380-85. PubMed Central, doi:10.1128/AAC.00055-06.
Collins, L. Torrero M., and Franzblau, S. Antimicrob. Agents Chemother. 1998, 42, 344-347.
Collins, L., and S. G. Franzblau. “Microplate Alamar Blue Assay versus BACTEC 460 System for High-Throughput Screening of Compounds against Mycobacterium Tuberculosis and Mycobacterium Avium.” Antimicrobial Agents and Chemotherapy, vol. 41, no. 5, May 1997, pp. 1004-09.
Falzari, Kanakeshwari, et al. “In Vitro and in Vivo Activities of Macrolide Derivatives against Mycobacterium Tuberculosis.” Antimicrobial Agents and Chemotherapy, vol. 49, no. 4, April 2005, pp. 1447-54. PubMed, doi:10.1128/AAC49.4.1447-1454.2005.
Franz, Nicholas D., et al. “Design, Synthesis and Evaluation of Indole-2-Carboxamides with Pan Anti-Mycobacterial Activity.” Bioorganic & Medicinal Chemistry, vol. 25, no. 14, 15 2017, pp. 3746-55. PubMed, doi:10.1016/j.bmc.2017.05.015.
Kondreddi, Ravinder Reddy, et al. “Design, Synthesis, and Biological Evaluation of Indole-2-Carboxamides: A Promising Class of Antituberculosis Agents.” Journal of Medicinal Chemistry, vol. 56, no. 21, November 2013, pp. 8849-59. PubMed, doi:10.1021/jm4012774.
Rao Srinivasa P. S., et al. “Indolcarboxamide Is a Preclinical Candidate for Treating Multidrug-Resistant Tuberculosis.” Science Translational Medicine, vol. 5, no. 214, December 2013, p. 214ra168. PubMed, doi:10.1126/scitranslmed.3007355.
Stec, Jozef, et al. “Indole-2-Carboxamide-Based MmpL3 Inhibitors Show Exceptional Antitubercular Activity in an Animal Model of Tuberculosis Infection.” Journal of Medicinal Chemistry, vol. 59, no. 13, July 2016, pp. 6232-47. ACS Publications, doi:10.1021/acs.jmedchem.6b00415.

It is to be understood that the invention is not limited to the particular embodiments of the invention described above, as variations of the particular embodiments may be made and still fall within the scope of the appended claims.

The invention will be further described, without limitation, by the following numbered paragraphs:

1. A compound of Formula (I):

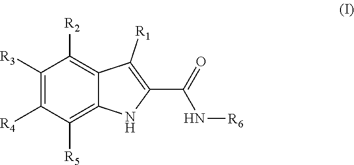

wherein:

R₁ is hydrogen, lower alkyl, or halogen; and

R₂ is hydrogen, lower alkyl, halo, cyano, trifluoromethyl, halo-lower alkyl, di-halo-lower alkyl, lower alkoxy, —OCH₂CH₂OCH₃, or carboxamide; and

R₃ is hydrogen, lower alkyl, halo, cyano, trifluoromethyl, halo-lower alkyl, di-halo-lower alkyl, lower alkoxy, —OCH₂CH₂OCH₃, or carboxamide; and

R₄ is hydrogen, lower alkyl, halo, cyano, trifluoromethyl, halo-lower alkyl, di-halo-lower alkyl, alkoxy, —OCH₂CH₂OCH₃, —(O(CH₂)ₘₘ)ₙₙ-morpholinyl, piperidinyl, carboxamide, ((C₁-C₄)alkyl)NH—, or (phenyl)NH—, where mm is 1 or 2 and nn is 0 or 1; or

R₃ and R₄ taken together with the aromatic carbon atoms to which they are attached form a fused 1,3-dioxolo; and

R₅ is hydrogen, lower alkyl, halo, cyano, trifluoromethyl, halo-lower alkyl, di-halo-lower alkyl, alkoxy, or carboxamide; and

R₆ is

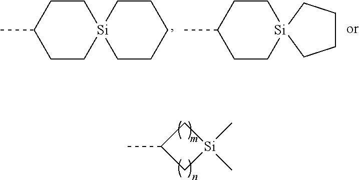

and

m is 1, 2 or 3,

n is 1, 2, 3, or 4; or

R₆ is

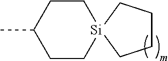

and m is 1 or 2;

or a pharmaceutically acceptable salt thereof.

2. The compound according to paragraph 1, or a pharmaceutically acceptable salt thereof, wherein R₁ is hydrogen or methyl or a pharmaceutically acceptable salt thereof.

3. The compound according to paragraph 2, or a pharmaceutically acceptable salt thereof, wherein R₁ is hydrogen.

4. The compound according to any one of paragraphs 1-3, or a pharmaceutically acceptable salt thereof, wherein R₂ is hydrogen, methyl, halogen, cyano, trifluoromethyl, or methoxy.

5. The compound according to paragraph 4, or a pharmaceutically acceptable salt thereof, wherein R₂ is methyl, halogen, cyano or methoxy.

6. The compound according to any one of paragraphs 1-5, or a pharmaceutically acceptable salt thereof, wherein R₃ is hydrogen, methyl, halogen, cyano, trifluoromethyl or methoxy.

7. The compound according to any one of paragraphs 1-5, or a pharmaceutically acceptable salt thereof, wherein R₃ is hydrogen, methyl, or methoxy.

8. The compound according to paragraph 7, or a pharmaceutically acceptable salt thereof, wherein R₃ is hydrogen.

9. The compound according to any one of paragraphs 1-8, or a pharmaceutically acceptable salt thereof, wherein R₄ is hydrogen, methyl, halogen, cyano, trifluoromethyl or methoxy.

10. The compound according to paragraph 9, or a pharmaceutically acceptable salt thereof, wherein R₄ is methyl or halogen.

11. The compound according to any one of paragraphs 1-8, or a pharmaceutically acceptable salt thereof, wherein R₄ is hydrogen, methyl, halogen, cyano, trifluoromethyl, methoxy, —(O(CH₂)ₘₘ)ₙₙ-morpholinyl, piperidinyl, ((C₁-C₄)alkyl)NH—, or (phenyl)NH—, where mm is 1 or 2 and nn is 0 or 1.

12. The compound according to any one of paragraphs 1-8, or a pharmaceutically acceptable salt thereof, wherein R₄ is hydrogen, lower alkyl, halo, cyano, trifluoromethyl, halo-lower alkyl, di-halo-lower alkyl, alkoxy, or carboxamide.

13. The compound according to any one of paragraphs 1-12, or a pharmaceutically acceptable salt thereof, wherein R₅ is hydrogen, methyl, halogen, cyano, trifluoromethyl or methoxy.

14. The compound according to paragraph 13, or a pharmaceutically acceptable salt thereof, wherein R₅ is hydrogen.

15. The compound according to any one of paragraphs 1-14, or a pharmaceutically acceptable salt thereof, wherein R₆, is

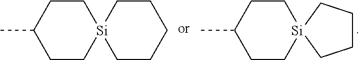

16. The compound according to any one of paragraphs 1-14, or a pharmaceutically acceptable salt thereof, wherein R₆ is

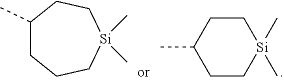

17. The compound according to any one of paragraphs 1-14, or a pharmaceutically acceptable salt thereof, wherein R₆NH is:

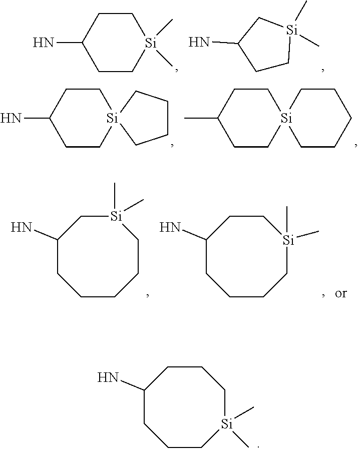

18. A compound according to claim 1 which is:
N-(1,1-dimethylsilinan-4-yl)-6-fluoro-4-methoxy-1H-indole-2-carboxamide;
N-(1,1-dimethylsilinan-4-yl)-4,6-difluoro-1H-indole-2-carboxamide;
N-(1,1-dimethylsilinan-4-yl)-4,6-difluoro-1H-indole-2-carboxamide;
4,6-difluoro-N-(5-silaspiro[4.5]decan-8-yl)-1H-indole-2-carboxamide;
4,6-dichloro-N-(5-silaspiro[4.5]decan-8-yl)-1H-indole-2-carboxamide;
N-(1,1-dimethylsilepan-4-yl)-4,6-dimethyl-1H-indole-2-carboxamide;
4,6-dimethyl-N-(5-silaspiro[4.5]decan-8-yl)-1H-indole-2-carboxamide;
4,6-dimethyl-N-(6-silaspiro[5.5]undecan-3-yl)-1H-indole-2-carboxamide;
ethyl N-(1,1-dimethylsilepan-4-yl)-4,6-difluoro-1H-indole-2-carboxamide;
N-(1,1-dimethylsilolan-3-yl)-4,6-dimethyl-1H-indole-2-carboxamide;
N-[(3R)-1,1-dimethylsilolan-3-yl]-4,6-dimethyl-1H-indole-2-carboxamide;
N-[(3S)-1,1-dimethylsilolan-3-yl]-4,6-dimethyl-1H-indole-2-carboxamide;
N-(1,1-dimethylsilocan-5-yl)-4,6-dimethyl-1H-indole-2-carboxamide;
N-(1,1-dimethylsilinan-4-yl)-4,6-dimethyl-1H-indole-2-carboxamide;
N-(1,1-dimethylsilepan-4-yl)-4,6-bis(trifluoromethyl)-1H-indole-2-carboxamide;
N-(1,1-dimethylsilocan-4-yl)-4,6-bis(trifluoromethyl)-1H-indole-2-carboxamide;
N-(1,1-dimethylsilocan-5-yl)-4,6-bis(trifluoromethyl)-1H-indole-2-carboxamide;
N-(1,1-dimethylsilolan-3-yl)-4,6-bis(trifluoromethyl)-1H-indole-2-carboxamide;
4,6-dichloro-N-(1,1-dimethylsilinan-4-yl)-3-methyl-1H-indole-2-carboxamide;
N-(1,1-dimethylsilinan-4-yl)-4,6-difluoro-3-methyl-1H-indole-2-carboxamide;
methyl N-(1,1-dimethylsilinan-4-yl)-4,6-bis(trifluoromethyl)-1H-indole-2-carboxamide;
4-bromo-N-(1,1-dimethylsilinan-4-yl)-6-methyl-1H-indole-2-carboxamide;
6-bromo-N-(1,1-dimethylsilinan-4-yl)-4-methyl-1H-indole-2-carboxamide;
4-bromo-N-(1,1-dimethylsilinan-4-yl)-6-(trifluoromethyl)-1H-indole-2-carboxamide;
6-cyano-N-(1,1-dimethylsilinan-4-yl)-4-methyl-1H-indole-2-carboxamide;
4-cyano-N-(1,1-dimethylsilinan-4-yl)-6-methyl-1H-indole-2-carboxamide;
4,6-dichloro-N-(1-methyl-3-trimethylsilyl-propyl)-1H-indole-2-carboxamide;
N-(1,1-dimethylsilinan-4-yl)-6-fluoro-4-isopropoxy-1H-indole-2-carboxamide;
N-(1,1-dimethylsilinan-4-yl)-6-fluoro-4-(2-methoxyethoxy)-1H-indole-2-carboxamide;
4,6-dichloro-N-(1,1-dimethylsilolan-3-yl)-1H-indole-2-carboxamide;
(R)-4,6-dichloro-N-(1,1-dimethylsilolan-3-yl)-1H-indole-2-carboxamide;
(S)-4,6-dichloro-N-(1,1-dimethylsilolan-3-yl)-1H-indole-2-carboxamide;
N-(1,1-dimethylsilolan-3-yl)-4,6-difluoro-1H-indole-2-carboxamide;
N-(1,1-dimethylsilinan-4-yl)-6-methyl-4-(trifluoromethyl)-1H-indole-2-carboxamide;
N-(1,1-dimethylsilocan-5-yl)-6-methyl-4-(trifluoromethyl)-1H-indole-2-carboxamide;
N-(1,1-dimethylsilinan-4-yl)-4-fluoro-6-methyl-1H-indole-2-carboxamide;
4,6-difluoro-N-(6-silaspiro[5.5]undecan-3-yl)-1H-indole-2-carboxamide; or
4,6-dichloro-N-(6-silaspiro[5.5]undecan-3-yl)-1H-indole-2-carboxamide,

or a pharmaceutically acceptable salt thereof.

19. A compound according to claim 1 which is:
N-(1,1-dimethylsilinan-4-yl)-6-fluoro-4-methoxy-1H-indole-2-carboxamide;
N-(1,1-dimethylsilinan-4-yl)-4,6-difluoro-1H-indole-2-carboxamide;
N-(1,1-dimethylsilinan-4-yl)-4,6-difluoro-1H-indole-2-carboxamide;
4,6-difluoro-N-(5-silaspiro[4.5]decan-8-yl)-1H-indole-2-carboxamide;
4,6-dichloro-N-(5-silaspiro[4.5]decan-8-yl)-1H-indole-2-carboxamide;
4,6-dimethyl-N-(5-silaspiro[4.5]decan-8-yl)-1H-indole-2-carboxamide;
4,6-dimethyl-N-(6-silaspiro[5.5]undecan-3-yl)-1H-indole-2-carboxamide;
N-(1,1-dimethylsilepan-4-yl)-4,6-difluoro-1H-indole-2-carboxamide;
N-(1,1-dimethylsilolan-3-yl)-4,6-dimethyl-1H-indole-2-carboxamide;
N-[(3R)-1,1-dimethylsilolan-3-yl]-4,6-dimethyl-1H-indole-2-carboxamide;
N-[(3S)-1,1-dimethylsilolan-3-yl]-4,6-dimethyl-1H-indole-2-carboxamide;
N-(1,1-dimethylsilocan-5-yl)-4,6-dimethyl-1H-indole-2-carboxamide;
N-(1,1-dimethylsilepan-4-yl)-4,6-bis(trifluoromethyl)-1H-indole-2-carboxamide;
N-(1,1-dimethylsilocan-5-yl)-4,6-bis(trifluoromethyl)-1H-indole-2-carboxamide;
N-(1,1-dimethylsilolan-3-yl)-4,6-bis(trifluoromethyl)-1H-indole-2-carboxamide;
4,6-dichloro-N-(1,1-dimethylsilinan-4-yl)-3-methyl-1H-indole-2-carboxamide;
N-(1,1-dimethylsilinan-4-yl)-4,6-bis(trifluoromethyl)-1H-indole-2-carboxamide;
4-bromo-N-(1,1-dimethylsilinan-4-yl)-6-methyl-1H-indole-2-carboxamide;
6-bromo-N-(1,1-dimethylsilinan-4-yl)-4-methyl-1H-indole-2-carboxamide;
6-cyano-N-(1,1-dimethylsilinan-4-yl)-4-methyl-1H-indole-2-carboxamide;
4-cyano-N-(1,1-dimethylsilinan-4-yl)-6-methyl-1H-indole-2-carboxamide;
N-(1,1-dimethylsilinan-4-yl)-6-fluoro-4-(2-methoxyethoxy)-1H-indole-2-carboxamide;
N-(1,1-dimethylsilinan-4-yl)-6-methyl-4-(trifluoromethyl)-1H-indole-2-carboxamide;
4,6-dichloro-N-(1,1-dimethylsilolan-3-yl)-1H-indole-2-carboxamide;
(R)-4,6-dichloro-N-(1,1-dimethylsilolan-3-yl)-1H-indole-2-carboxamide;
(S)-4,6-dichloro-N-(1,1-dimethylsilolan-3-yl)-1H-indole-2-carboxamide;
N-(1,1-dimethylsilolan-3-yl)-4,6-difluoro-1H-indole-2-carboxamide;
N-(1,1-dimethylsilinan-4-yl)-4-fluoro-6-methyl-1H-indole-2-carboxamide;
4,6-difluoro-N-(6-silaspiro[5.5]undecan-3-yl)-1H-indole-2-carboxamide; or
4,6-dichloro-N-(6-silaspiro[5.5]undecan-3-yl)-1H-indole-2-carboxamide.

20. A pharmaceutical composition, comprising a compound of any one of paragraphs 1-19, or a pharmaceutically acceptable salt thereof, and one or more pharmaceutically acceptable carriers and/or additives.

21. The pharmaceutical composition according to paragraph 20, further comprising one or more additional anti-infective agents.

22. The pharmaceutical composition according to paragraph 21, wherein said additional anti-infective agent is rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin or levofloxacin, or combinations thereof.

23. A method of treating a mycobacterial infection, comprising the step of administering a therapeutically effective amount of a compound of any one of paragraphs 1-19, or a pharmaceutically acceptable salt thereof, to a patient in need thereof.

24. The method of paragraph 23, wherein the mycobacterial infection is caused by Mycobacterium tuberculosis, Mycobacterium avium, Mycobacterium kansasii, Mycobacterium abscessus or Mycobacterium chelonae.

25. The method of paragraph 23, wherein the mycobacterial infection is caused by Mycobacterium tuberculosis.

26. The method of any one of paragraphs 23-25, wherein the patient is afflicted with tuberculosis (TB), multi-drug-resistant tuberculosis (MDR-TB), pre-extensively drug resistant (Pre-XDR-TB) or extensively drug-resistant tuberculosis (XDR-TB).

It is to be understood that the invention is not limited to the particular embodiments of the invention described above, as variations of the particular embodiments may be made and still fall within the scope of the appended claims.

What is claimed is:

1. A compound of Formula (I): 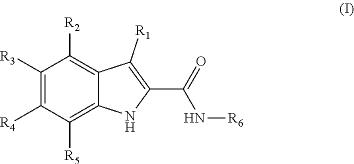
wherein:
R₁ is hydrogen, lower alkyl, or halogen; and
R₂ is hydrogen, lower alkyl, halo, cyano, trifluoromethyl, halo-lower alkyl, di-halo-lower alkyl, lower alkoxy, —OCH₂CH₂OCH₃, or carboxamide; and
R₃ is hydrogen, lower alkyl, halo, cyano, trifluoromethyl, halo-lower alkyl, di-halo-lower alkyl, lower alkoxy, —OCH₂CH₂OCH₃, or carboxamide; and
R₄ is hydrogen, lower alkyl, halo, cyano, trifluoromethyl, halo-lower alkyl, di-halo-lower alkyl, alkoxy, —OCH₂CH₂OCH₃, —(O(CH₂)ₘₘ)ₙₙ-morpholinyl, piperidinyl, ((C₁-C₄)alkyl)NH—, or (phenyl)NH—, where mm is 1 or 2 and nn is 0 or 1 or carboxamide; or
R₃ and R₄ taken together with the aromatic carbon atoms to which they are attached form a fused 1,3-dioxolo; and
R₅ is hydrogen, lower alkyl, halo, cyano, trifluoromethyl, halo-lower alkyl, di-halo-lower alkyl, alkoxy, or carboxamide; and
R₆ is 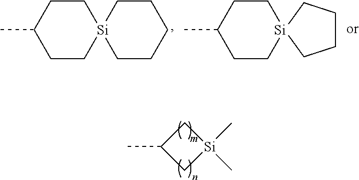
and
m is 1, 2 or 3, and n is 1, 2, 3, or 4;
or a pharmaceutically acceptable salt thereof.

2. The compound according to claim 1, or a pharmaceutically acceptable salt thereof, wherein R₁ is hydrogen or methyl or a pharmaceutically acceptable salt thereof.

3. The compound according to claim 2, or a pharmaceutically acceptable salt thereof, wherein R₁ is hydrogen.

4. The compound according to claim 1, or a pharmaceutically acceptable salt thereof, wherein R₂ is hydrogen, methyl, halogen, cyano, trifluoromethyl, or methoxy.

5. The compound according to claim 4, or a pharmaceutically acceptable salt thereof, wherein R₂ is methyl, halogen, cyano or methoxy.

6. The compound according to claim 1, or a pharmaceutically acceptable salt thereof, wherein R₃ is hydrogen, methyl, halogen, cyano, trifluoromethyl or methoxy.

7. The compound according to claim 6, or a pharmaceutically acceptable salt thereof, wherein R₃ is hydrogen, methyl, or methoxy.

8. The compound according to claim 7, or a pharmaceutically acceptable salt thereof, wherein R₃ is hydrogen.

9. The compound according to claim 1, or a pharmaceutically acceptable salt thereof, wherein R₄ is hydrogen, methyl, halogen, cyano, trifluoromethyl or methoxy.

10. The compound according to claim 9, or a pharmaceutically acceptable salt thereof, wherein R₄ is methyl or halogen.

11. The compound according to claim 1, or a pharmaceutically acceptable salt thereof, wherein R₄ is hydrogen, methyl, halogen, cyano, trifluoromethyl, methoxy, —(O(CH₂)ₘₘ)ₙₙ-morpholinyl, piperidinyl, ((C₁-C₄)alkyl)NH—, or (phenyl)NH—, where mm is 1 or 2 and nn is 0 or 1.

12. The compound according to claim 1, or a pharmaceutically acceptable salt thereof, wherein R₄ is hydrogen, lower alkyl, halo, cyano, trifluoromethyl, halo-lower alkyl, di-halo-lower alkyl, alkoxy, or carboxamide.

13. The compound according to claim 1, or a pharmaceutically acceptable salt thereof, wherein R₅ is hydrogen, methyl, halogen, cyano, trifluoromethyl or methoxy.

14. The compound according to claim 13, or a pharmaceutically acceptable salt thereof, wherein R₅ is hydrogen.

15. The compound according to claim 1, or a pharmaceutically acceptable salt thereof, wherein R₆, is

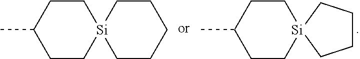

16. The compound according to claim 1, or a pharmaceutically acceptable salt thereof, wherein R₆ is

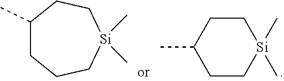

17. The compound according to claim 1, or a pharmaceutically acceptable salt thereof, wherein R₆NH is:

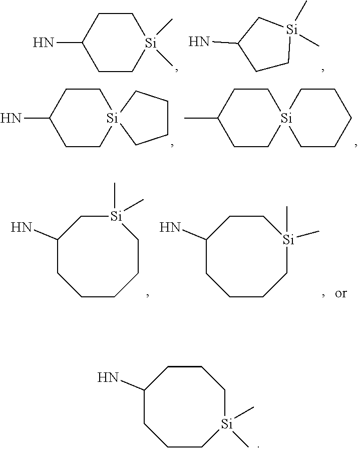

18. A compound according to claim 1 which is:
N-(1,1-dimethylsilinan-4-yl)-6-fluoro-4-methoxy-1H-indole-2-carboxamide;
N-(1,1-dimethylsilinan-4-yl)-4,6-difluoro-1H-indole-2-carboxamide;
N-(1,1-dimethylsilinan-4-yl)-4,6-difluoro-1H-indole-2-carboxamide;
4,6-difluoro-N-(5-silaspiro[4.5]decan-8-yl)-1H-indole-2-carboxamide;
4,6-dichloro-N-(5-silaspiro[4.5]decan-8-yl)-1H-indole-2-carboxamide;
N-(1,1-dimethylsilepan-4-yl)-4,6-dimethyl-1H-indole-2-carboxamide;
4,6-dimethyl-N-(5-silaspiro[4.5]decan-8-yl)-1H-indole-2-carboxamide;
4,6-dimethyl-N-(6-silaspiro[5.5]undecan-3-yl)-1H-indole-2-carboxamide;
ethyl N-(1,1-dimethylsilepan-4-yl)-4,6-difluoro-1H-indole-2-carboxamide;
N-(1,1-dimethylsilolan-3-yl)-4,6-dimethyl-1H-indole-2-carboxamide;
N-[(3R)-1,1-dimethylsilolan-3-yl]-4,6-dimethyl-1H-indole-2-carboxamide;
N-[(3S)-1,1-dimethylsilolan-3-yl]-4,6-dimethyl-1H-indole-2-carboxamide;
N-(1,1-dimethylsilocan-5-yl)-4,6-dimethyl-1H-indole-2-carboxamide;
N-(1,1-dimethylsilinan-4-yl)-4,6-dimethyl-1H-indole-2-carboxamide;
N-(1,1-dimethylsilepan-4-yl)-4,6-bis(trifluoromethyl)-1H-indole-2-carboxamide;
N-(1,1-dimethylsilocan-4-yl)-4,6-bis(trifluoromethyl)-1H-indole-2-carboxamide;
N-(1,1-dimethylsilocan-5-yl)-4,6-bis(trifluoromethyl)-1H-indole-2-carboxamide;
N-(1,1-dimethylsilolan-3-yl)-4,6-bis(trifluoromethyl)-1H-indole-2-carboxamide;
4,6-dichloro-N-(1,1-dimethylsilinan-4-yl)-3-methyl-1H-indole-2-carboxamide;
N-(1,1-dimethylsilinan-4-yl)-4,6-difluoro-3-methyl-1H-indole-2-carboxamide;
methyl N-(1,1-dimethylsilinan-4-yl)-4,6-bis(trifluoromethyl)-1H-indole-2-carboxamide;
4-bromo-N-(1,1-dimethylsilinan-4-yl)-6-methyl-1H-indole-2-carboxamide;
6-bromo-N-(1,1-dimethylsilinan-4-yl)-4-methyl-1H-indole-2-carboxamide;
4-bromo-N-(1,1-dimethylsilinan-4-yl)-6-(trifluoromethyl)-1H-indole-2-carboxamide;
6-cyano-N-(1,1-dimethylsilinan-4-yl)-4-methyl-1H-indole-2-carboxamide;
4-cyano-N-(1,1-dimethylsilinan-4-yl)-6-methyl-1H-indole-2-carboxamide;
4,6-dichloro-N-(1-methyl-3-trimethylsilyl-propyl)-1H-indole-2-carboxamide;
N-(1,1-dimethylsilinan-4-yl)-6-fluoro-4-isopropoxy-1H-indole-2-carboxamide;
N-(1,1-dimethylsilinan-4-yl)-6-fluoro-4-(2-methoxyethoxy)-1H-indole-2-carboxamide;
4,6-dichloro-N-(1,1-dimethylsilolan-3-yl)-1H-indole-2-carboxamide;
(R)-4,6-dichloro-N-(1,1-dimethylsilolan-3-yl)-1H-indole-2-carboxamide;
(S)-4,6-dichloro-N-(1,1-dimethylsilolan-3-yl)-1H-indole-2-carboxamide;
N-(1,1-dimethylsilolan-3-yl)-4,6-difluoro-1H-indole-2-carboxamide;
N-(1,1-dimethylsilinan-4-yl)-6-methyl-4-(trifluoromethyl)-1H-indole-2-carboxamide;
N-(1,1-dimethylsilocan-5-yl)-6-methyl-4-(trifluoromethyl)-1H-indole-2-carboxamide;
N-(1,1-dimethylsilinan-4-yl)-4-fluoro-6-methyl-1H-indole-2-carboxamide;
4,6-difluoro-N-(6-silaspiro[5.5]undecan-3-yl)-1H-indole-2-carboxamide; or
4,6-dichloro-N-(6-silaspiro[5.5]undecan-3-yl)-1H-indole-2-carboxamide,
or a pharmaceutically acceptable salt thereof.

19. A compound according to claim 1 which is:
N-(1,1-dimethylsilinan-4-yl)-6-fluoro-4-methoxy-1H-indole-2-carboxamide;
N-(1,1-dimethylsilinan-4-yl)-4,6-difluoro-1H-indole-2-carboxamide;
N-(1,1-dimethylsilinan-4-yl)-4,6-difluoro-1H-indole-2-carboxamide;
4,6-difluoro-N-(5-silaspiro[4.5]decan-8-yl)-1H-indole-2-carboxamide;
4,6-dichloro-N-(5-silaspiro[4.5]decan-8-yl)-1H-indole-2-carboxamide;
4,6-dimethyl-N-(5-silaspiro[4.5]decan-8-yl)-1H-indole-2-carboxamide;
4,6-dimethyl-N-(6-silaspiro[5.5]undecan-3-yl)-1H-indole-2-carboxamide;
N-(1,1-dimethylsilepan-4-yl)-4,6-difluoro-1H-indole-2-carboxamide;
N-(1,1-dimethylsilolan-3-yl)-4,6-dimethyl-1H-indole-2-carboxamide;
N-[(3R)-1,1-dimethylsilolan-3-yl]-4,6-dimethyl-1H-indole-2-carboxamide;
N-[(3S)-1,1-dimethylsilolan-3-yl]-4,6-dimethyl-1H-indole-2-carboxamide;
N-(1,1-dimethylsilocan-5-yl)-4,6-dimethyl-1H-indole-2-carboxamide;
N-(1,1-dimethylsilepan-4-yl)-4,6-bis(trifluoromethyl)-1H-indole-2-carboxamide;
N-(1,1-dimethylsilocan-5-yl)-4,6-bis(trifluoromethyl)-1H-indole-2-carboxamide;
N-(1,1-dimethylsilolan-3-yl)-4,6-bis(trifluoromethyl)-1H-indole-2-carboxamide;
4,6-dichloro-N-(1,1-dimethylsilinan-4-yl)-3-methyl-1H-indole-2-carboxamide;
N-(1,1-dimethylsilinan-4-yl)-4,6-bis(trifluoromethyl)-1H-indole-2-carboxamide;
4-bromo-N-(1,1-dimethylsilinan-4-yl)-6-methyl-1H-indole-2-carboxamide;
6-bromo-N-(1,1-dimethylsilinan-4-yl)-4-methyl-1H-indole-2-carboxamide;
6-cyano-N-(1,1-dimethylsilinan-4-yl)-4-methyl-1H-indole-2-carboxamide;
4-cyano-N-(1,1-dimethylsilinan-4-yl)-6-methyl-1H-indole-2-carboxamide;
N-(1,1-dimethylsilinan-4-yl)-6-fluoro-4-(2-methoxyethoxy)-1H-indole-2-carboxamide;
N-(1,1-dimethylsilinan-4-yl)-6-methyl-4-(trifluoromethyl)-1H-indole-2-carboxamide;
4,6-dichloro-N-(1,1-dimethylsilolan-3-yl)-1H-indole-2-carboxamide;
(R)-4,6-dichloro-N-(1,1-dimethylsilolan-3-yl)-1H-indole-2-carboxamide;
(S)-4,6-dichloro-N-(1,1-dimethylsilolan-3-yl)-1H-indole-2-carboxamide;
N-(1,1-dimethylsilolan-3-yl)-4,6-difluoro-1H-indole-2-carboxamide;
N-(1,1-dimethylsilinan-4-yl)-4-fluoro-6-methyl-1H-indole-2-carboxamide;
4,6-difluoro-N-(6-silaspiro[5.5]undecan-3-yl)-1H-indole-2-carboxamide; or
4,6-dichloro-N-(6-silaspiro[5.5]undecan-3-yl)-1H-indole-2-carboxamide,
or a pharmaceutically acceptable salt thereof.

20. A pharmaceutical composition, comprising a compound according to claim 1, or a pharmaceutically acceptable salt thereof, and one or more pharmaceutically acceptable carriers and/or additives.

21. The pharmaceutical composition according to claim 20, further comprising one or more additional anti-infective agents.

22. The pharmaceutical composition according to claim 21, wherein said additional anti-infective agent is rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin or levofloxacin, or combinations thereof.

23. A method of treating a mycobacterial infection, comprising the step of administering a therapeutically effective amount of a compound according to claim 1, or a pharmaceutically acceptable salt thereof, to a patient in need thereof.

24. The method according to claim 23, wherein the mycobacterial infection is caused by Mycobacterium tuberculosis, Mycobacterium avium, Mycobacterium kansasii, Mycobacterium abscessus or Mycobacterium chelonae.

25. The method according to claim 23, wherein the mycobacterial infection is caused by Mycobacterium tuberculosis.

26. The method according to claim 23, wherein the patient is afflicted with tuberculosis (TB), multi-drug-resistant tuberculosis (MDR-TB), pre-extensively drug resistant (Pre-XDR-TB) or extensively drug-resistant tuberculosis (XDR-TB).
